(19) United States
(12) Patent Application Publication
(10) Pub. No.: US 2015/0368181 A1
(43) Pub. Date: Dec. 24, 2015

(54) RECYCLABLE CHIRAL CATALYST FOR ASYMMETRIC NITROALDOL REACTION AND PROCESS FOR THE PREPARATION THEREOF

(71) Applicant: COUNCIL OF SCIENTIFIC 7 INDUSTRIAL RESEARCH, New Delhi (IN)

(72) Inventors: Rukhsana Ilyas KURESHY, Bhavnagar (IN); Noor-Ul-Hasan KHAN, Bhavnagar (IN); Sayed Hasan Razi ABDI, Bhavnagar (IN); Hari Chand BAJAJ, Bhavnagar (IN); Tamal ROY, Bhavnagar (IN); Anjan DAS, Bhavnagar (IN)

(73) Assignee: Council of Scientific & Industrial Research, New Delhi (IN)

(21) Appl. No.: 14/764,833

(22) PCT Filed: Oct. 31, 2013
(86) PCT No.: PCT/IN2013/000670

§ 371 Date: Jul. 30, 2015

(30) Foreign Application Priority Data
Feb. 1, 2013 (IN) 0289/DEL/2013

(51) Int. Cl. C07C 201/12; C07F 1/08; C07F 15/02; C07F 15/06; C07F 11/00; B01J 31/18; C07F 3/06

(52) U.S. Cl. CPC C07C 201/12; B01J 31/1805; C07F 1/08; C07F 3/06; C07F 15/06; C07F 11/00; C07F 15/02; B01J 2231/342; B01J 2531/16; B01J 2531/26; B01J 2531/842; B01J 2531/845; B01J 2531/62

(57) ABSTRACT

The present invention relates to preparation of highly efficient chiral recyclable homogeneous catalysts generated in situ by the reaction of chiral oligomeric [H₄] ligands and a metal salt taken in 1:1 molar ratio for asymmetric nitroaldol reaction, wherein nitroaldol reactions of various aldehydes such as aromatic, aliphatic α,β-unsaturated aldehydes, alicyclic aldehydes and nitroalkenes were carried out to produce optically active β-nitroalcohols in high yield and with moderate to excellent enantioselectivity (ee up to >95%) in presence of a base and an optically active chiral recyclable homogeneous catalyst represented by the following formula (I).

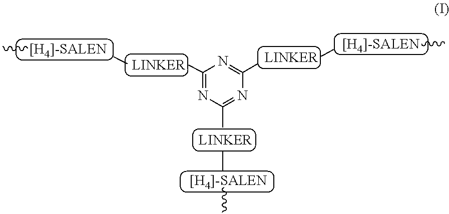

The following specification particularly describes the nature of the invention and the manner in which it is to be performed.

### FIELD OF THE INVENTION

The present invention relates to recyclable chiral catalysts for asymmetric nitroaldol reaction for the synthesis of pharmaceutically important compounds. Particularly, present invention relates to a process for the preparation of recyclable chiral catalysts. More particularly, present invention demonstrates the use of chiral oligomeric [H₄]salen Cu(II) complexes as homogeneous catalysts for the nitroaldol reaction of aldehydes, which produces optically pure nitro alcohols. Optically pure nitro alcohols are important intermediates in the preparation of chiral molecules of pharmaceuticals.

### BACKGROUND OF THE INVENTION

Reference may be made to J. Tian et al., in Angew. Chem. Int. Ed., 41 (19) (2002) 3636 which reported a multifunctional (YLi₃-{tris(binaphthoxide)}) single catalyst component where a proper chiral environment was created by the use of achiral additives. These catalysts gave nitroaldol products in 51-84% yield with ee 11-62% at −40° C. in case of simple aromatic aldehydes. The drawbacks of this process are; (i) catalytic system needs very low temperature (−40° C.) to get reasonably high yield and moderate ee of the product; (ii) separation of the catalyst from the product is difficult (iii) catalyst is non-recyclable.

Reference may be made to S. Handa et al., in Angew. Chem. Int. Ed., 47 (2008) 3230 which discloses the use of dinucleating heterobimetallic Pd/La/Schiff base complexes in catalytic asymmetric nitroaldol reaction of various aldehydes with nitroethane providing nitroaldol products in 25-92% yield and ee in 1-92% which on reduction gave â-amino alcohols. The drawbacks of this process are; (i) reaction takes very long time (69-120 h) to complete; (ii) catalytic reaction requires very low temperature (about −40° C.); (iii) expensive metal source like palladium and lanthanum were used; (iv) catalyst is non-recyclable.

Reference may be made to K. Iseki et al., in Tetrahedron Letters, 37 (1996) 9081 which reported rare earth-lithium-BINOL complexes catalyzed asymmetric nitroaldol reaction of α,α′-difluoro aldehydes with nitromethane at lower temperature (−40° C.). Catalytic reaction gave product yield in 55-82% and ee in 55-94%. The drawbacks of this process are; (i) completion of reaction takes very long time (96 h); (ii) catalytic reaction needs very low temperature (−40° C.); (iii) expensive metal sources like lanthanum; samarium europium, ytterbium and gadolinium were used; (iv) catalyst is non-recyclable.

Reference may be made to D. A. Evans et al., in J. Am. Chem. Soc., 125 (2003) 12692 which discloses the synthesis of a series of chiral bidentate bis oxazoline ligands with divalent metal acetates which were used as enantioselective catalysts for the nitroaldol process. In this process indabox ligand in combination with Cu(OAc)₂ was found to be the best catalyst for the nitroaldol reaction of various aldehydes to give products in 66-95% and ee in 89-94%. The drawback of this process is (i) the catalyst is non-recyclable.

Reference may be made to A. P. Bhatt et al., in J. Mol. Cat. A, 244 (2006) 110 which describes the synthesis of covalently attached heterogeneous catalyst based on La—Li-BINOL-silica and La—Li-BINOL-MCM-41 for enantioselective nitroaldol reaction of aldehydes. Asymmetric nitroaldol reaction with these complexes gave the product in 0-94% yield and ee in 55-90%. The drawbacks of this process are; (i) catalytic system requires very lower temperature (−40° C.); (ii) expensive metal like La is used to prepare active catalyst.

Reference may be made to R. Kowalczyk et al., in Tetrahedron: Asymmetry, 18 (2007) 2581, which describes asymmetric nitroaldol reaction of aldehydes catalyzed by Cr(III)-salen with 1,2-diaminocyclohexane and 1,2-diphenylethylenediamine collar. Salen complex catalyzed nitroaldol reaction gave products in good yield with moderate ee (40-76%) at −78° C. in 20-96 h. The drawbacks of this process are; (i) the reaction needed extremely low temperature (−78° C.) (ii) catalyst is non-recyclable (iii) enantioselectivity is moderate (ee, 40-76%).

Reference may be made to W. Mansawat et al, in Tetrahedron Letters, 48 (2007) 4235, which reported novel synthesis of chiral thiolated amino-alcohols based ligands for Cu-catalyzed asymmetric nitroaldol reaction of aldehydes. Thiolated amino-alcohols in combination with Cu(OAc)₂ were screened as a potential catalysts for nitroaldol reaction of aromatic aldehydes to give product in 69-92% yield and ee in 69-88%. The drawbacks of this process are; (i) good performance of the catalyst is limited to the substrates benzaldehydes having electron-withdrawing groups; (ii) catalyst is non-recyclable.

Reference may be made to B. M. Choudary et al., in J. Am. Chem. Soc., 127 (2005) 13167, which describes the nano crystalline MgO in combination with (S)-BINOL as a recyclable heterogeneous catalyst at −78° C. for the asymmetric nitroaldol reaction to give chiral nitro alcohols in 70-95% yield and 60-98% ee. The drawback of this process is; (i) extremely low temperature (−78° C.) is required to show high activity and enantioselectivity of the catalytic system.

Reference may be made to G. Zi et al., Inorg. Chim. Acta, 361 (2008) 1246 which reports the preparation, structure, and catalytic activity of chiral (S,S)-1,2-diamino cyclohexane-based N₄-donor ligands in combination with Cu(II) metal ion as catalyst for asymmetric nitroaldol reaction of benzaldehyde in presence of triethyl amine to give moderate to high yield (75-95%) of the product but with poor enantioselectivity (4-29%) at 0 to −20° C. The drawbacks of this process are; (i) low enantioselectivity of the product; (ii) catalysts are non-recyclable; (iii) low temperatures (0 to −20° C.) were used.

Reference may be made to Z. Zhang et al., Inorg. Chim. Acta, 362 (2009) 1687, which reports the preparation, structure, and catalytic activity of chiral cis-3-aminoazetidines in combination with different Cu(II) salts for asymmetric nitroaldol reaction of aromatic aldehydes to give the products in 8-85% yield with 12-51% ee at 0° C. The drawbacks of this process are; (i) catalysts are non-recyclable; (ii) Low to moderate conversions with low enantioselectivity were achieved at lower temperature 0° C.

Reference may be made to G. Blay et al., in Tetrahedron: Asymmetry, 17 (2006) 2046, which reports the preparation of chiral iminopyridine ligands in a modular fashion from monoterpenic (camphor-derived) ketones and pyridinylalkylamines. The in situ generated complex with Cu(OAc)₂.H₂O was used for the enantioselective nitroaldol reaction of o-anisol at lower temperature (−65° C.) in presence of triethylamine giving 56% yield of the corresponding product with 86% ee in 36-72 h. The drawbacks of this process are; (i) ligands are not recyclable; (ii) extremely low temperature (−65° C.) is required to get ee up 86% with good yield (iii) long reaction time (36-72 h).

Reference may be made to U.S. Pat. No. 5,616,726 which discloses a process for the nitroaldol of (S)-3-phenyl-2-phthaloylaminopropanal to get 3-amino-2-hydroxy-4-phenylbutyric acid derivatives as immunopotentiating anticancer agent using lanthanum/(S)-1,1′-bi-2-naphthol complexes as catalyst at 0 to −50° C. in 2-72 h. The drawbacks of this process are; (i) 1N HCl is required to remove the ligand from reaction mixture; (ii) expensive complex of lanthanum/(S)-1,1-bi-2-naphthol was not recycled; (iii) requires very low temperature (−50° C.) for best results; (iv) reaction requires long time for completion (72 h).

Reference may be made to U.S. Pat. No. 6,632,955 which discloses the Ln—Li-BINOL complex as catalyst for asymmetric nitroaldol reaction for the preparation of optically active nitro alcohol derivatives. The drawbacks of this process are; (i) reaction time is more (67 h); (ii) for best performance the system needs very low temperature (−40° C.); (iii) 1N aqueous solution of hydrochloric acid (HCl) was used to separate the products from the reaction mixture; (iv) the catalyst is not recyclable.

Reference may be made to U.S. Pat. No. 6,977,315 which discloses a process for asymmetric nitroaldol reaction using N,N′-bis[2-(2,4,6-trimethylbenzoyl)-3-oxobutylidene]-(1S,2S)-bis(3,5-dimethylphenyl)ethylene-1,2-diaminato cobalt (II) complexes as catalysts in the presence of a base producing optically active nitro alcohols at very low temperature (−70° C.). The drawbacks of this process are; (i) reaction time is more (76 h); (ii) for good performance reaction has to be done at very low temperature (−70° C.); (iii) the catalyst is not recyclable.

Reference may be made to U.S. Pat. No. 5,336,653 which discloses the preparation of chiral catalyst based on lanthanum derived from dilithium salt of 2,2′-dihydroxy-1,1′-binaphthyl and a trivalent lanthanum chloride under anhydrous conditions to give β-hydroxynitro compound in good yield and ee at −42° C. The drawbacks of this process are; (i) catalyst preparation requires more time (3 days) (ii) system is substrate specific and works better only with cyclohexylaldehyde as substrate, while both conversion and ee is moderate with other substrates; (ii) catalytic system needs lower temperature (−42° C.).

Reference may be made to WO Patent No: 2010084772 which discloses the preparation of chiral catalyst based on lanthanum derived from disodium salt of (S)-2-fluoro-N-(1-((4-fluoro-2-hydroxyphenyl)amino)-4-methyl-1-oxopentan-2-yl)-5-hydroxybenzamide and a trivalent lanthanum chloride under anhydrous conditions to give desired product anti-1,2-nitroalkanol in very high yield with a high anti-selection. The anti-1,2-nitroalkanolare intermediates for an optically active anti-1,2-aminoalcohol. The drawbacks of this process is; (i) catalyst works better only at lower temperature (−40° C.).

Reference may be made to CN Patent No: 101773856 which discloses the preparation of chiral copper complex with amido oxazoline Schiff base and its use in asymmetric nitroaldol reaction of aldehydes. The catalytic system showed better catalytic activity under mild reaction conditions. The drawback of this process is; (i) catalyst is not recyclable.

Reference may be made to JP Patent No: 2008044928 which discloses the preparation of chiral Cu(II)-imidazotine based catalyst for asymmetric nitroaldol reaction of aldehydes using nitromethane at RT. The drawbacks of this process are (i) catalysts synthesis is multi-step.

Reference may be made to CN Patent No: 20101182476 which discloses the preparation of C₂ symmetric chiral bisoxazoline ligands containing imidazole salt ion pair group. In situ generated complexes of chiral bisoxazoline ligands with Cu(II) and Zn(II) metal ions were used for asymmetric nitroaldol reaction of aldehydes. High yield and enantioselectivity in the product was achieved with fifteen times catalyst recyclability. The drawbacks of this process is (i) ligands synthesis is multi-steps. Ligands are synthesized by multi-step process.

Reference may be made to K. Ma and J. Youin Chem. Eur. J. 13 (2007) 1863, which discloses the rational design of sterically and electronically easily tunable bi-functional chiral bisimidazolines. The in situ generated complexes with Cu(OTf)₂ were used as efficient catalysts in highly enantioselective nitroaldol reaction of aromatic and aliphatic aldehydes in presence of triethylamine. The drawback of this process are; (i) ligand preparation requires expensive starting materials and the resulting metal complexes used as catalysts are not recyclable.

Reference may be made to G. Blay et al., in Chem. Commun, (2006) 4840-4842, which describes the synthesis of camphor-derived C₁-symmetric amino pyridineligand and its in situ generated complex with Cu(OAc)₂.H₂O in presence of diisopropylamine at −40° C. was used as catalyst for the synthesis of highly enantiomerically enriched 2-bromo-2-nitroalkan-1-ols by direct condensation of aliphatic and aromatic aldehydes with bromonitromethane. The catalytic system gave 99% yield of the corresponding nitroaldol product with 97% enantioselectivity. The main drawbacks of this process are; (i) recycling of expensive ligand is not demonstrated; (ii) ee with good yield was achieved only at extremely low temperatures.

Reference may be made to C. J. Cooper et al.; in Dalton Trans. 40(2011) 3677-3682, which has reported the synthesis and crystal structure of a series of pyridine based Cu(II) complexes for enantioselective nitroaldol reaction of benzaldehyde as substrate at 0° C. in presence of triethylamine as base. High conversion to β-nitroalcohol with 84% enantioselectivity was achieved. But the main limitation of the system is (i) the catalytic system was not recyclable (ii) enantioselectivity was obtained up to 84% at 0° C. (iii) the catalyst was used for nitroaldol reaction of only benzaldehyde as substrate with nitromethane.

Reference may be made to F. Bure\check{s} et al.; in Tetrahedron Letters. 50(2009) 3042-3045, has designed a series of camphor-imidazoline ligands and their in situ generated complexes with Copper acetate were used as catalysts for enantioselective nitroaldol reaction of 4-nitrobenzaldehyde at RT. Limitation of the system is (i) High yield (94%) of the product with was obtained only with moderate enantioselectivity (up to 67%). (ii) the catalytic system was not recyclable (iii) catalytic experiments were performed only with 4-nitrobenzaldehyde as substrate.

Reference may be made to W. Yang et al.; in Eur. J. Org. Chem. (2011) 1552-1556 have reported a 16-member library of C₂-symmetric modular chiral BINOL-oxazoline Schiff base copper(II) complexes generated in situ in a one-pot, three-component manner for the asymmetric nitroaldol reaction of aldehydes. This modular catalyst library was evaluated in the asymmetric nitroaldol reaction, for which excellent yields (up to 98%) and enantioselectivities (up to 98% ee) were obtained under mild conditions. The drawbacks of the system are (i) catalysts are not recyclable and reusable (ii) the catalyst synthesis is multistep.

Reference may be made to J.-J. Jiang et al. Tetrahedron Asymmetry 18 (2007) 1376-1382, have described the synthesis of chiral phosphine-salen type ligand, derived from (R)-2-(diphenylphosphino)-1,10-binaphthyl-2′-amine and used as fairly effective chiral ligand for Cu(I)-promoted enantioselective nitroaldol reactions of arylaldehydes with nitromethane to give the corresponding adducts in moderate enantioselectivities and moderate to good yield. Main limitations of this catalytic system are (i) moderate enantioselectivity and yield of the respective products are achieved (ii) High catalyst loading to give the corresponding adducts (iii) catalyst is not recyclable.

Reference may be made to M. Steurer et al. in J. Org. Chem. 2010, 75 3301-3310, have described the preparation of amino-functionalized sulfonimidamides via aziridinium ring-opening reactions and nucleophilic substitutions of sulfonimidoyl chlorides. In situ generated Cu(I) complex with sulfonimidamides as ligands were used as catalyst in asymmetric nitroaldol reactions of aromatic aldehydes with nitromethane to give products with enantioselectivities up to 95% ee with good yields. The drawbacks of this catalytic system are (i) system is not recyclable (ii) need high catalyst loading.

Reference may be made to K. Y. Spangler et al. Org. Lett. 11, 2009 4724-4727, have reported bisoxazolidine as an effective ligand in the Me₂Zn-promoted and the Cu(I)-catalyzed nitroaldol reaction. While a wide range of nitroaldol products are obtained in high yields and ee in both the cases, the replacement of dimethylzinc with copper(I) acetate results in a complete reversal of the sense of asymmetric induction. The Cu(I)-catalyzed enantioselective addition of nitromethane to methyl 4-oxobutanoate followed by hydrogenation and spontaneous lactamization gives (S)-5-hydroxypiperidin-2-one in 72% overall yield and 98% ee. The Drawbacks of the system are (i) high catalyst loading (ii) lower reaction temperature and (iii) catalysts are not recyclable.

Reference may be made to I. Panov et al. in J. Org. Chem. 76(2011), 4787-4793, who has reported ten optically pure substituted 2-(pyridin-2-yl)imidazolidin-4-ones. The absolute configurations of individual ligands were determined by X-ray analysis or NOESY experiments. The Cu(II) complexes of the respective ligands were studied as enantioselective catalysts of the nitroaldol reaction of aldehydes with nitromethane, giving the corresponding substituted 2-nitroalkanols in 24-72 h. In the case of an anti arrangement of the imidazolidin-4-one ring, the obtained result was 91-96% ee, whereas in the case of syn arrangement, a significant drop in the ee (25-27%) was observed. Limitation of the system is (i) catalysts are not recyclable and (ii) long reaction time in most of the substrates used (30-72 h).

Reference may be made to S. K. Ginotra et al. in Org. Biomol. Chem., 2007, 5, 3932-3937 reported the synthesis of a C₂-symmetric diethyl ⁱPr-bis(oxazoline)-Cu(OAc)₂.H₂O catalyst for enantioselective nitroaldol reaction between nitromethane and various aldehydes to provide β-hydroxy nitroalkanes with high chemical yields (up to 95%) and enantiomeric excesses (up to 97%). The main drawbacks of this process are; (i) recycling of expensive ligand is not demonstrated; (ii) high catalyst loading to obtain the above mentioned selectivity and yield.

Reference may be made to Y. Xion et al. in Chem. Eur. J. 13(2007), 829-833 have reported chiral hydrogenated salen-(CuOTf)₂.C₇H₈ (10 mol %) catalyst for the asymmetric nitroaldol reaction of aromatic, heteroaromatic, enal, and aliphatic aldehydes in presence of 4 Å molecular sieves which produces the expected products in moderate to high yields (up to 98%) with excellent enantioselectivities (up to 96% ee). This process is successfully extended to the synthesis of (S)-norphenylephrinein 67% overall yield, starting from commercially available m-hydroxybenzaldehyde. Limitation of the system is (i) catalysts recyclability was not demonstrated.

Reference may be made to Y. Zhou et al. in J. Org. Chem. 76(2011), 588-600 has reported a small library of C₁-symmetric chiral diamines Ligands via condensing exo-(−)-bornylamine or (+)-(1S,2S,5R)-menthylamine with various Cbz-protected amino acids. Among them, only one ligand/CuCl₂.2H₂O complex (2.5 mol %) showed outstanding catalytic efficiency for asymmetric nitroaldol reaction between a variety of aldehydes and nitroalkanes to afford the expected products in high yields (up to 98%) with excellent enantioselectivities (up to 99%) and moderate to good diastereoselectivities (up to 90:10). This process is air- and moisture tolerant and has been applied to the synthesis of (S)-2-amino-1-(3,4-dimethoxyphenyl)ethanol, a key intermediate for (S)-epinephrine and (S)-norepinephrine. The drawbacks of the system are (i) Catalyst preparation is elaborated and multistep (ii) catalyst is not recyclable and (ii) lower temperature (4 to −20° C.) is required for catalytic reaction to achieve higher yield and enantioselectivity.

Reference may be made to M. Bandini et al. in Chem. Commun., 2007, 616-618 has disclosed remarkable generality in scope of bis amino-Cu catalysts for enantioselective nitroaldol for a range of aldehydes giving high chiral induction (ee 81-99%). The present catalytic system is the key step for the preparation of highly functionalized tetrahydro-isoquinolines. The drawbacks of the system are (i) catalyst is not recyclable and (ii) for best results in term of product yield and ee needs lower reaction temperature (0° C.).

Reference may be made to D. Didier et al. in Adv. Synth. Catal. 353(2011), 1087-1095 has reported an anthracenyl-modified chiral bis(oxazoline) copper complex to promote nitroaldol reactions between structurally varying aldehydes and nitromethane or nitroethane to give products in 46-90% with ee 4-91%. The drawbacks of the system are (i) the catalyst synthesis and loading on silica is multistep (ii) The catalyst leached significantly in each recycle experiment (iii) long reaction time (36-120 h).

Reference may be made to F. Tur et al. in Org. Lett., 9(2007) 24 has reported the synthesis of 3,3′-bis-diethylaminomethyl-2,2′-dihydroxy-1,1′-dinaphthalene and its chiral monometallic lanthanum(III) triflate salt was used as catalyst for catalytic enantioselective nitroaldol reaction of simple □-trifluoromethyl ketones with nitromethane. The resulting □-trifluoromethyl tertiary nitroaldols were obtained in moderate to high yields (up to 93%) and enantioselectivities (up to 98% ee) in 96 h at −40° C. These adducts are versatile chiral building blocks and may be reduced (NiCl₂/NaBH₄) to their β-amino-α-trifluoromethyl tertiary alcohols without toss of enantiomeric purity. The drawbacks of the system are (i) the catalyst is not recyclable, (ii) for achieving best yield and ee in the products the nitroaldol reaction has to be performed at lower temperature −40° C., (iii) reaction time is more (96 h).

Reference may be made to A. Noole et al. in J. Org. Chem. 75(2010), 1313-1316 has reported a complex derived from the enantiomeric bipiperidine and copper(II) acetate hydrate as an efficient catalyst for the enantioselective nitroaldol reaction in presence of triethylamine as base. An excellent result in term of yield and enantiomeric excess was achieved at −25° C. The drawbacks of the system are (i) the catalyst is not recyclable, (ii) for achieving best yield and ee in the products the nitroaldol reaction has to be performed at lower temperature (−25° C.).

Reference may be made to W. Jin et al. in Chem. Eur. J. 16(2010), 8259-8261 has described the preparation of bis(sulfonamide)-diamine-type ligand and its in situ generated complex was used as catalyst for asymmetric nitroaldol reaction of aromatic and aliphatic aldehydes. The catalyst system demonstrates excellent performance, providing up to 99% ee for the synthesis of β-hydroxynitroalkanes with aliphatic aldehydes. The drawbacks of the system are (i) the catalyst is not recyclable.

Reference may be made to T. Nitabaru et al. in J. Am. Chem. Soc. 131(2009), 13860-13869 has described the synthesis of heterogeneous Nd/Na heterobimetallic complex comprised of Nd₅O(OⁱPr)₁₃, an amide-based ligand, and NaHMDS (sodium hexamethyldisilazide) for an anti-selective catalytic asymmetric nitroaldol reaction of aldehydes. The catalytic system worked well under heterogeneous conditions, affording the corresponding 1,2-nitroalkanol in a highly anti-selective (up to anti/syn) >40/1) and enantioselective manner (up to 98% ee) at −40° C. The drawbacks of the system are (i) For achieving the best results in term of yield and enantioselectivity the catalytic nitroaldol reaction has to conducted at lower temperature (−40° C.).

Reference may be made to B. Qin et al. in J. Org. Chem. 72(2007) 9323-9328 has reported the preparation of chiral N,N′-dioxide-CuI catalyst with spacer of various length for the asymmetric nitroaldol reaction of both aromatic and heteroaromatic aldehydes. The corresponding nitro-alcohol products were obtained in good yields with high enantiomeric excess (up to 99% ee) at −40° C. The drawbacks of the system are (i) the catalyst is not recyclable, (ii) for achieving the best results in term of yield and enantioselectivity the catalytic nitroaldol reaction has to conducted at lower temperature (−45° C.).

Reference may be made to N. H. Khan et al. in Catal Lett. 140(2010) 189-196 has described the synthesis of C₂-symmetric chiral secondary bis-amines based ligand derived from 1,2-diaminocyclohexane structure having H, t-Bu and Cl substituents and their in situ generated Cu(II) complexes were used as catalysts for an environmentally benign protocol for highly enantioselective nitroaldol reaction of various aldehydes with nitromethane in the presence of different ionic liquids as a greener reaction medium at 0° C. High yields (78-90%) of β-nitroalcohols with high enantioselectivity (ee, 81-94%) was achieved when [emim]BF₄ was used as ionic liquid with catalyst recyclability up to five cycles. The drawbacks of the system are (i) for achieving the best results in term of yield and enantioselectivity the catalytic nitroaldol reaction has to conducted at lower temperature (0° C.); (the catalytic system requires 12 mol % ligand and 10 mol % copper salt for best results; (iii) the catalyst is recyclable only in ionic liquid media; (iv) the reaction takes 30 h to complete.

Reference may be made to H. Y. Kim et al. in Org. Lett., 11(2009) 24 has reported the use of brucine derived aminoalcohol to generate Cu(I) and Zn(II) metal complexes as active catalysts for asymmetric nitroaldol reaction of various aldehydes. Both the catalytic system worked well to give the products in moderate to high yield (45-96%) and low to high enantioselectivity (2-95%) in 18-40 h at −15° C. The drawbacks of the system are (i) the catalyst is not recyclable; (ii) low reaction temperature (−15° C.) is needed to achieve best performance of the catalysts (iii) long reaction time in some cases (40 h).

Reference may be made to C. Christensen et al. in J. Org. Chem. 67(2002), 4875-4881, who has reported the catalytic enantioselective Henry reaction of α-keto esters with nitromethane using copper(II)-tert-butyl bisoxazoline complex in combination with triethylamine as base to give optically active β-nitro-α-hydroxy esters in high yields and with excellent enantiomeric excesses at lower temperature (−24° C.). The scope of the reaction is demonstrated by converting the β-nitro-α-hydroxy esters into Boc-protected β-amino-α-hydroxy esters in high yields and without loss of optical purity. Drawbacks of the catalytic system (i) very high catalyst loading (20 mol %) (ii) catalyst is not recyclable, (iii) reaction temperature is very low (−24° C.).

Reference may be made to H. Maheswaran et al. in Chem. Commun., 2006, 4066-4068 has described the synthesis of diacetatato[(−)-sparteine-N,N′]copper(II), and dichloro[(−)-sparteine-N,N′]copper(II) for asymmetric nitroaldol reaction of various aldehydes and nitromethane. High yield of the product with enantioselectivities (73-97% ee) was achieved when the reaction is done at 0° C. Drawbacks of the system are (i) high catalyst loading (20 mol %) (ii) low reaction temperature (0° C.) (iii) catalysts were not recyclable.

Reference may be made to S. Selvakumar et al. in Org. Biomol. Chem., 7(2009), 3156-3162 has reported a copper(II) complex of C₂-symmetric aminoacid derived diamine as efficient catalysts for the enantiosetective nitroaldol reaction between nitroalkanes and various aldehydes in presence of triethylamine to provide β-hydroxy nitroalkanes in high yields (up to 97%), moderate diastereoselectivities (up to 71:29) and excellent enantiomeric excesses (ee up to 96%). This protocol was further used to convert the chiral nitroaldol adduct into chiral aziridine in few steps. Drawbacks of the system are (i) very low reaction temperature (−40° C., (ii) catalysts are not recyclable.

Reference may be made to C. Christensen et al. in Chem. Commun., 2001, 2222-2223 has described the development of chiral bisoxazoline-copper(II) complexes as potential catalyst for enantiosetective nitroaldol reaction of various □□keto esters with nitromethane. High yield of the product with enantioselectivities (5-93% ee) was achieved when the reaction is done at 50° C. Drawbacks of the system are (i) reaction occur at higher temperature (50° C.); (ii) catalyst is not recyclable.

Reference may be made to T. Risgaard et al. in Org. Biomol. Chem., 1(2003), 153-156 have screened a series of chiral Cu(II) complexes with different chiral ligands viz., BINAP, 1,2-bis((2S,5S)-2,5-dimethylphospholano)benzene (DUPHOS)(P,P-chelating), bisoxazolines (BOX) and Py-BOX (N,N-chelating), and phosphinoxazoline (N,P-chelating) and dibenzofuradiylphenyloxazoline (DBFOX) catalyzed asymmetric nitroaldol reaction of aldehydes with silyl nitronates. The best yield, diastereo- and enantioselectivity of the nitroalcohols formed are obtained by the application of a copper(II)-diphenyl-bisoxazoline complex as the catalyst in the presence of tetrabutylammonium triphenylsilyldifluorosilicate (TBAT). Drawbacks of the catalytic system (i) high catalyst loading (20 mol %) (ii) catalysts are not recyclable; (iii) the protocol requires expensive TBAT in order to show high efficiency.

Reference may be made to J. Gao et al. in Org. Biomol. Chem., 1(2003), 2801-2806 has reported the synthesis of dimeric and trimeric macrocyclic Schiff base ligands derived 2,5-thiophenedicarboxaldehyde with 1R,2R-diaminocyclohexane. The reduced form of these macrocyclic ligands in combination with Zn(II) metal ion were used as active catalyst for asymmetric nitroaldol reaction of aldehydes at −20° C. The results in term of yield (19-68%) and enantiomeric excess (21-75%) of the product was achieved in THF as solvent for benzaldehyde. Drawbacks of the catalytic system (i) Catalyst is not recyclable (ii) long reaction time (48 h)(iii) only demonstrated with substrate benzaldehyde; (iv) moderate yield and ee of the product was achieved.

Reference may be made to S. Liu et al. in Org. Lett., 10(2008), 1831-1834, they have reported a C₂-symmetric bis oxazolidine-Zn(II) complex catalyzed asymmetric nitroaldol reaction of aliphatic and aromatic aldehydes. β-Hydroxy nitroalkanes were produced in up to 99% yield and 95% ee in 9 h at −15° C. Drawbacks of the catalytic system is (i) low reaction temperature (−15° C.) (ii) catalyst is not recyclable.

Reference may be made to G. Zhang et al. in Adv. Synth. Catal. 351(2009), 1255-1262 which reports the synthesis of chiral Cu(II)-supramolecular metal-organic frameworks and used it as catalyst for asymmetric nitroaldol and aza-Henry reactions of aromatic and aliphatic aldehydes and N-protected aromatic imines in high yield (51-96%) and good to excellent enantioselectivity (73-99%) at 0° C.-room temperature for 1.5-72 h under the optimized condition. Reactions can be performed in the absence of a base in ethanol or water. Drawback is (i) The catalyst is not recyclable.

Reference may be made to B. M. Trost et al. in Angew. Chem. Int. Ed. 41(2002), 861-863, which has reported the development of dinuclear zinc complex based on chiral semi-aza-crown ligand. This catalyst has been successfully applied in enantioselective nitroaldol reaction of aromatic and heterocyclic aldehydes as substrate in presence of nitromethane at −35 to −78° C. Drawbacks of the catalytic system is (i) low reaction temperature (−35 to −78° C.) (ii) catalyst is not recyclable.

Reference may be made to WO Patent No. 2011110895A1 discloses the synthesis of chiral Cu(II) amino alcohols covalently attached on to mesoporous silica as heterogeneous catalyst for asymmetric nitroaldol reaction of aldehydes which produces optically pure nitro alcohols in high yield and enantioselectivity in presence of chiral imine. The drawbacks of the systems are (i) multi-step synthesis of the catalyst, (ii) best results in term of yield and enantioselectivity were achieved when chiral imine is used as an additive.

Reference may be made to Y.-g. Gao et al. in Russian Journal of Organic Chemistry, 43(2007), 1754-1756 which has reported the synthesis of copper-Schiff base complexes derived from salicylaldehydes and amino alcohols with copper(II) acetate monohydrate. The complexes were used as effective catalysts in the asymmetric nitroaldol reaction affording nitro alkanols in up to 98% yield with moderate enantiomeric excess (up to ee 38.6%). The Drawbacks of this system are (i) catalysts are not recyclable; (ii) enantioselectivity is not at desirable level (only up to 38.6%).

Reference may be made to Wen-Jing Xiao et al. Chem. Commun., 2012, 48, 5596-5598, have developed a new class of chiral sulfoxide-Schiff base ligands, The in situ generated copper complex with these ligands were found to be highly efficient for asymmetric nitroaldol reaction of aldehydes in term of yield (98%) and enantioselectivity (96%) under mild reaction conditions. Drawbacks of the system are (i) catalyst system is not recyclable. (ii) reaction time is very long for few substrate s (30-132 h).

Reference may be made to J. Guo et al. in Chirality 21(2009) 619-627, have reported chiral bi-functional ONO salen copper-based catalytic system derived from aminoaldohols with aldehydes of various bulkiness for asymmetric nitro-aldol reactions between nitromethane and aldehydes, that afforded corresponding adducts with moderate to good yields and enantioselectivities at 50° C. Drawbacks of the catalytic system are (i) catalysts are not recyclable (ii) temperature is higher (50° C.).

Reference may be made to B. M. Trost et al. in Org. Lett., 4(2002) 2621-2623, have reported synthesis of dinuclear zinc complexes based on ligand system derived from diarylcarbinol moiety and varied phenol units for the asymmetric nitroaldol reactions. Catalytic enantioselective nitroaldol reaction of aldehydes promoted by these modified ligands at −78° C. led to efficient syntheses of the β-receptor agonists (−)-denopamine and (−)-arbutamine. The drawbacks of the catalytic system are (i) need very low reaction temperature (−78° C.) (ii) catalysts are not recyclable.

Reference may be made to H. Sasai et al. in Tetrahedron Letters, 34(1993) 2657-2660, have reported various asymmetric rare earth complexes derived from rare earth metal trichlorides such as YCl₃, LaCl₃, PrCl₃, NdCl₃, SmCl₃, EuCl₃, GdCl₃, TbCl₃, YbCl₃ with dilithium (R)-binaphthoxide in combination with NaOH and H₂O. The optical purities (ee, 93%) of the nitroaldol product was obtained when the reaction was conducted at −50° C. that depend on the size of rare earth metals used. The catalyst system was used for the synthesis of (S)-metaprolol, which is a β₁-selective β-blocker. The main limitation of this catalytic system is (i) very low reaction temperature (−50° C.).

Reference may be made to H. Sasai et al. in Tetrahedron Letters. 35(1994) 6123-6126, has disclosed the use of a series of rare earth Li-BINOL complexes as catalysts in the asymmetric nitroaldol reaction of α-amino aldehydes and nitromethane. Among the various complexes used, La—Li-BINOL complex gave best results in term of yield (92%) and ee (99%) of the product in 72 h at −40° C. A typical adduct, (2S,3S)-3-phthaloylamino-2-hydroxy-1-nitro-4-phenylbutane was conveniently converted to (2S,3S)-3-amino-2-hydroxy-4-phenylbutanoic acid (erythro-AHPA; phenylnorstatine), a component of the HIV protease inhibitor KNI-227 and KNI-272. Limitations of the catalyst are (i) very low reaction temperature (−40° C.) and (ii) very long reaction time (72 h).

Reference may be made to K. Iseki et al. in Tetrahedron Letters. 37(1996) 9081-9084, have reported rare earth-Li-BINOL complexes to catalyze nitroaldol reaction of α,α-difluoroaldehydes. Among the complexes used, Sm—Li-BINOL complex gave the highest enantioselectivity (94%) at −40° C. The Drawbacks of this catalyst system are (i) very low reaction temperature (−40° C.) is required (ii) very long reaction time (168 h).

Reference may be made to Kureshy et al. ACS Catal., 1(2011), 1529-1535 have developed chiral monomeric and dimeric macrocyclic [H₄]salen ligands derived from 1R,2R-(−)-1,2-diaminocyclohexane and 1R,2R-(−)-1,2-diphenyl-1,2-diaminoethane with trigolbis aldehydes. Chiral Cu(II) complexes generated in situ by the interaction of different source of copper (II) salts with chiral monomeric and dimeric macrocyclic [H₄]salen were used for asymmetric nitroaldol reaction of aromatic and aliphatic aldehydes at RT. Excellent yields (45-98%) of β-nitroalcohols with high enantioselectivity (ee, 90-99%) was achieved in ca. 20 h with the use of chiral mononuclear and dinuclear macrocyclic Cu(II) salen complexes with diphenyldiamine collar. Both chiral mononuclear and dinuclear macrocyclic [H₄]salen catalysts mediated nitroaldol process is eight times recyclable. Drawbacks of this catalyst system is it needs (i) high catalyst loading (15 mol %) is required for high activity and enantioselectivity in the product.

Reference may be made to A. Gualandi et al. in J. Org. Chem. 76(2011), 3399-3408, have reported the synthesis of chiral perazamacrocycles containing four pyrrole rings by the [2+2] condensation of (R,R)-diaminocyclohexane and 5,50-(alkane-2,2-diyl)bis(1H-pyrrole-2-carbaldehydes). These macrocycles were used as ligands in the copper-catalyzed nitroaldol reactions of aromatic and aliphatic aldehydes with nitroalkanes. Methyl-substituted macrocyclic ligand in combination with copper diacetate in ethanol provided nitroaldol products of aromatic and aliphatic aldehydes with high yield (20-98%) enantiomeric excesses (43-95% ee) at room temperature. The drawbacks of this catalyst system is (i) catalyst is not recyclable.

Reference may be made to G. Lai et al. in Chem. Eur. J. 17(2011), 1114-1117, has reported the use of chiral proline based Cu(II) complexes in the asymmetric nitroaldol reaction of aromatic and aliphatic aldehydes to give the nitroaldol product in 81-99% yield with excellent ee 91-99% in 12-72 h in water. Drawbacks of the catalytic system are (i) lower reaction temperature (0° C.) (ii) catalytic system is not recyclable (iii) reaction time is high for most substrate (72 h).

Reference may be made to N. Demirel et al in CHIRALITY 23(2011), 374-378, have reported Chiral Schiff-bases based on ferrocene for the enantioselective Nitroaldol (Henry) reaction. The Schiff-bases were obtained-in shorter reaction times and improved yield under microwave irradiation method over classical method. The highest enantioselectivity was observed (95% ee) when CH₂Cl₂ was used as solvent. The main drawbacks of the catalytic system are (i) high catalyst loading (ii) not recyclable.

Reference may be made to K. Kodama et al. in Chem. Eur. J. 17(2011), 13584-13592, has reported the synthesis of different chiral 1,3-diamines, derived from (−)-cis-2-benzamidocyclohexanecarboxylic acid for their use in Cu-catalyzed asymmetric nitroaldol reaction between aromatic and aliphatic aldehydes and nitromethane at 0° C. to give β-nitroalcohols in excellent yields (up to 98%) and enantioselectivities (91%). Drawbacks of this catalyst system are (i) multi-step catalyst synthesis (iii) lower reaction temperature (0° C.) (iv) system is not recyclable.

Reference may be made to L. Cheng et al. in Chem. Eur. J. 16(2010), 6761-6765, has reported a highly enantioselective nitroaldol reaction between various aldehydes and nitroethane, catalyzed by chiral Cu(II)-bisimidazoline catalyst to give high yield (58-98%) having high selectivity for syn product of ee above 90%. Drawbacks of this catalyst system are (i) low reaction temperature (0° C.) (ii) system is not recyclable.

Reference may be made to M. J. Rodig et al. in Tetrahedron: Asymmetry 22 (2011) 1097-1102, has reported the synthesis of C₂-symmetric isoquinoline-based chiral diimine and various aldehydes giving 50-89% yield and 75-93% ee at 5-10 mot % catalyst loading in 24 h at room temperature. Drawback of this catalyst system is (i) catalyst synthesis requires expensive reagents to give the desired in overall low yield (ii) catalyst is not recyclable.

Reference may be made to R. Maggi et al. in Eur. J. Org. Chem. 2011, 5551-5554, has reported chiral 2,2′-methylenebis[(4S)-4-tertbutyl-2-oxazoline] incorporated in styrene-divinylbenzene crosslinked network as recyclable heterogeneous catalyst for the enantioselective nitroaldol reaction of nitromethane with substituted benzaldehydesin ethanol to give chiral β-nitroalcohols in 56-95% yield with 49-88% ee. Drawback of the catalytic system is (i). Since the core chiral ligand was present during polymerization step, it is difficult to get uniform distribution of the active center (ii) due to same reason maintaining structure of polymer in different synthesis batches is difficult.

Reference may be made to M. Luo et al. in Tetrahedron Letters 51 (2010) 5577-5580, has disclosed the synthesis of a series of N-metal complexes containing chiral □-ethylphenyl amines for asymmetric nitroaldol reaction to give products with high enantioselectivity (>99%). The Drawbacks are (i) high catalyst loading (15 mol %) (ii) long reaction time (72 h) (iii) system is not recyclable.

Kureshy et al. in Applied Catalysis A: General 439-440 (2012) 74-79 reported the use of chiral macrocyclic salen-[H₄] ligands with trigol linker (15 mol %) with Cu(II) ion (10 mol %) to catalyze enantioselective nitroaldol reaction of various aromatic and aliphatic aldehydes with nitromethane in the presence of several bases at room temperature to give the chiral nitro alcohol in yields (up to 92% with respect to the aldehyde) with high enantioselectivity (ee, ˜95%) in about 30 h. the catalyst system was recyclable up to 8 cycles with no significant loss in its performance. The main drawback of this system is (i) the catalyst requires additional base for the reaction to be effective; (ii) catalyst system requires 15 mol % of the chiral ligand with 10 mol % coppers salt for achieving higher product yield and ee.

The present invention relates to recyclable homogenous catalyst used for the nitroaldol reaction of aldehydes, which produces optically pure nitro alcohols. It is emphasized that in homogenous, all the reactive sites are available, hence the catalyst performance is better in terms of selectivity and is devoid of catalyst/metal leaching problem which is prevalent in heterogenous catalyst.

### OBJECTS OF THE INVENTION

The main objective of the present invention is to provide recyclable chiral catalyst useful for asymmetric nitroaldol reaction for the synthesis of pharmaceutically important compounds, which obviates the drawbacks as detailed above.

Another object of the present invention is to provide a process for the preparation of recyclable chiral catalysts for asymmetric nitroaldol reaction for the synthesis of pharmaceutically important compounds.

Yet another objective of the present invention is to provide a process for the preparation of recyclable chiral Cu(II)-oligomeric[H₄]salen complexes of optically pure diamine.

Another object of the present invention is to use these chiral catalysts for asymmetric nitroaldol reaction.

Yet another object of the present invention is to provide a process for the synthesis of oligimeric [H₄]salen copper complexes derived from optically pure diamines for asymmetric nitroaldol reaction to produce 1,2-nitroalcohols in high yield.

Still another object of the present invention is to use these oligimeric [H₄]salen copper complexes as catalysts for asymmetric nitroaldol reaction of various aldehydes, aromatic aldehydes, aliphatic aldehydes, α,β-unsaturated aldehydes and alicyclic aldehydes to obtain 1,2-nitroalcohols with enantioselectivity more than 95% ee.

Still another object of the present invention is to recycle the chiral homogeneous catalyst without loss in yield of final product and having enantioselectivity greater than 95%.

Still another object of the present invention is to provide a method wherein different inexpensive non-chiral bases are used as additives to produce chirally pure 1,2-nitro alcohols in high yield and excellent ee (>95%) at ambient temperature (25-27° C.). Still another object of the present invention is to prepare chirally pure 1,2-nitroalcohols using recyclable chiral Cu(II)-oligomeric [H₄]salen complex as homogeneous catalyst having catalyst loading less than <5 mol %.

Still another object of the present invention is to provide a method to produce chirally pure 1,2-nitro alcohols in high yield with excellent ee (>95%) at ambient temperature (25-27° C.) and convert some of these to pharmaceutically active compounds.

### SUMMARY OF THE INVENTION

Accordingly, present invention provides a chiral homogeneous catalyst comprising chiral ligand of general formula 1 along with metal

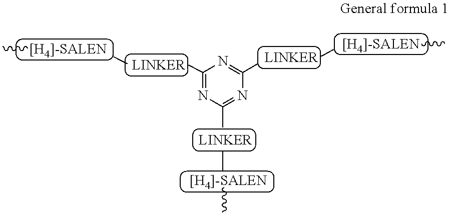

wherein
linker attached to melamine is selected from the group consisting of

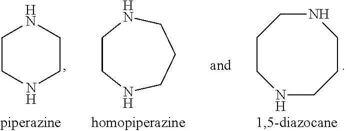

[H₄]salen attached to linker is selected from the group consisting of

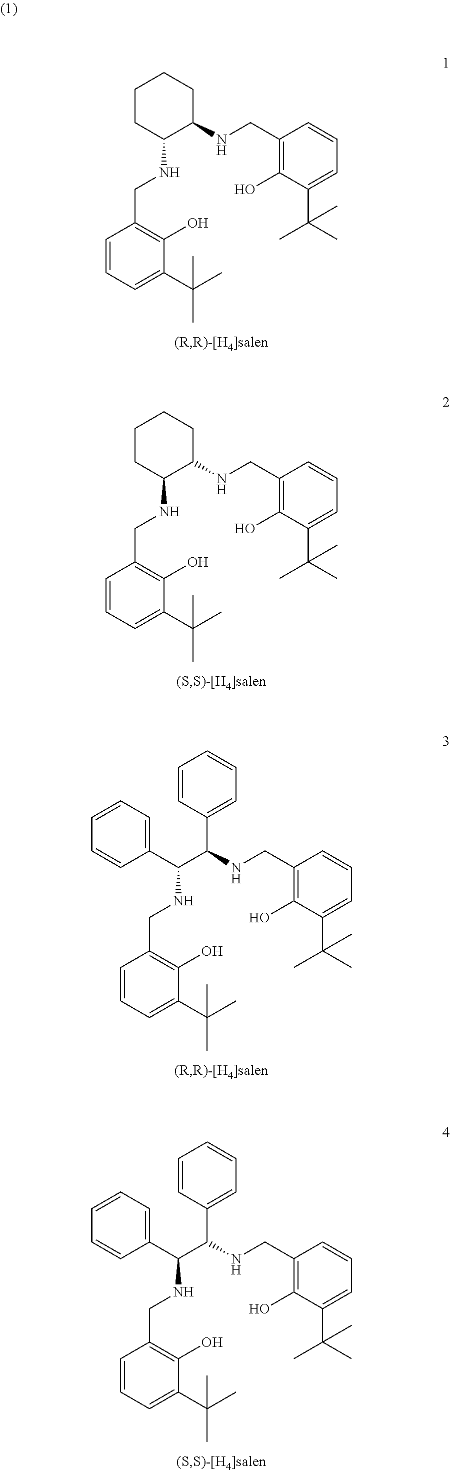

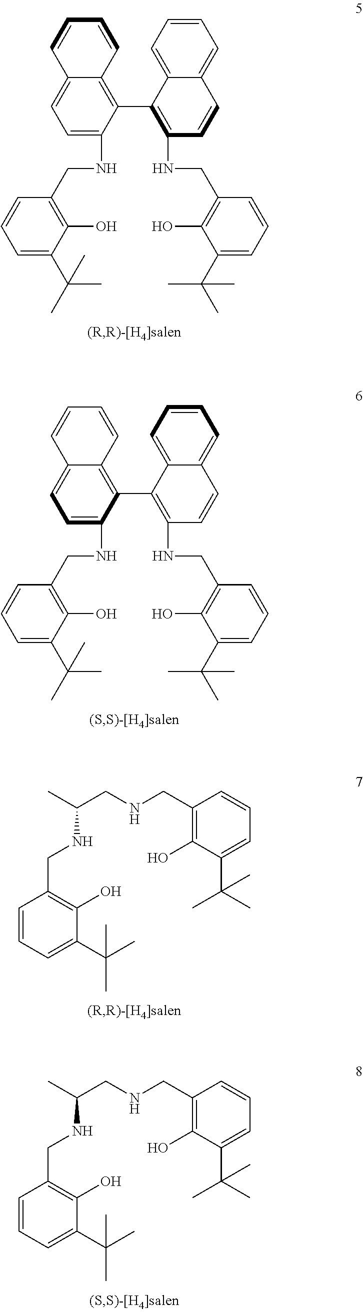

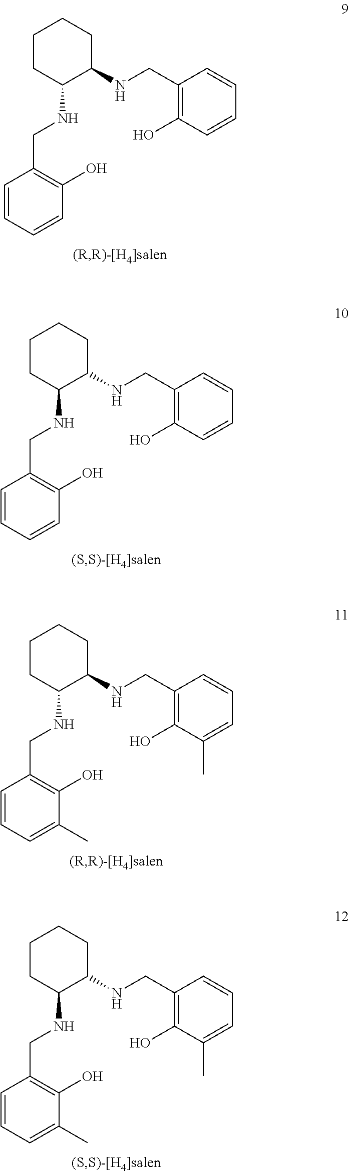

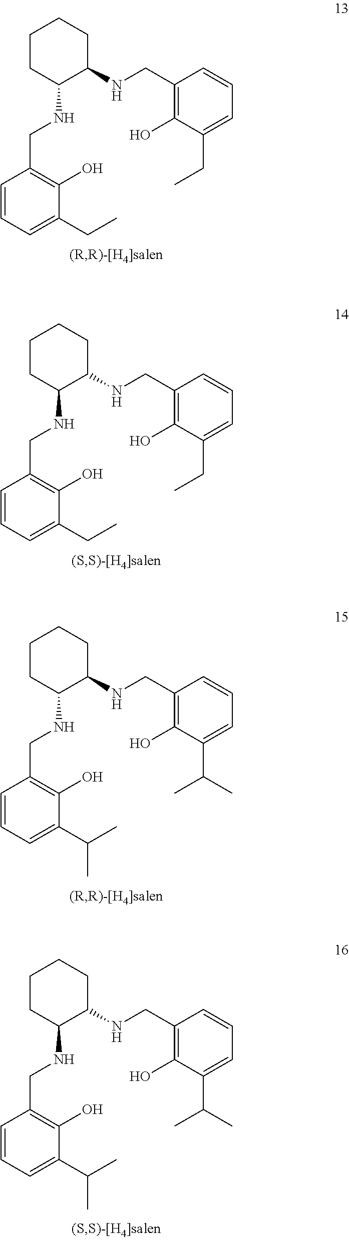

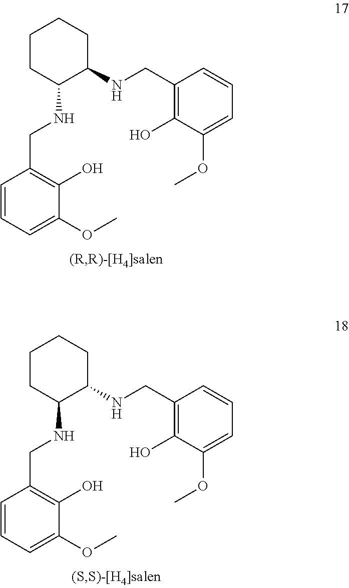 [SMILES: COc1cccc(CN[C@@H]2CCCC[C@H]2NCc2cccc(OC)c2O)c1O ; COc1cccc(CN[C@H]2CCCC[C@@H]2NCc2cccc(OC)c2O)c1O]

In an embodiment of the present invention, metal used is selected from the group consisting of cobalt(II), nickel (II), copper (I), copper (II) and Zn(II) preferably copper (II).

In one embodiment of the present invention, said catalyst is useful for asymmetric nitroaldol reaction for the synthesis of pharmaceutically important compounds.

In another embodiment of the present invention, chiral ligand of general formula 1 comprising:

piperazine: (1R,2R)—[H₄]salen 1; piperazine: (1S,2S)—[H₄]salen 2; piperazine: (1R,2R)—[H₄]salen 3; piperazine: (1S,2S)—[H₄]salen 4; piperazine: (R)—[H₄]salen 5; piperazine: (S)—[H₄]salen 6; piperazine: (R)—[H₄]salen 7; piperazine (S)—[H₄]salen 8; piperazine (1R,2R)—[H₄]salen 9; piperazine: (1S,2S)—[H₄]salen 10; piperazine: (1R,2R)—[H₄]salen 11; piperazine: (1S,2S)—[H₄]salen 12; piperazine: (1R,2R)—[H₄]salen 13; piperazine: (1S,2S)—[H₄]salen 14; piperazine: (1R,2R)—[H₄]salen 15; piperazine: (1S,2S)—[H₄]salen 16; piperazine: (1R,2R)—[H₄]salen 17; piperazine: (1S,2S)—[H₄]salen 18; homopiperazine: (1R,2R)—[H₄]salen 19; homopiperazine: (1S,2S)—[H₄]salen 20; 1,5-diazocane: (1R,2R)—[H₄]salen 21; 1,5-diazocane: (1S,2S)—[H₄]salen 22.

In yet another embodiment, present invention provides a process for the preparation of chiral ligand of formula 1 and the said process comprising the steps of:
i. reacting cyanuric chloride with a linker in the molar ratio ranging between 1:3 to 1:5 in the presence of 5 to 10 equivalent tertiary amine in dry tetrahydrofuran followed by refluxing under inert atmosphere for a period in the range of 12 to 24 h at temperature ranging from 65 to 66° C.;
ii. evaporating tetrahydrofuran from the reaction mixture as obtained from step (i) followed by extracting the solid thus obtained with dichloromethane, washing the dichloromethane layer with water, drying the dichloromethane layer with anhydrous sodium sulphate, evaporating of dichloromethane to give white solid and finally recrystalizing white solid from a mixture of dichloromethane and hexane (1:3);
iii. treating the white crystalline solid obtained from step (ii) with reagent in methanol in the molar ratio ranging between 1:20 to 1:40 at temperature in the range of 0 to 20° C. for period in the range of 3 to 8 h followed by keeping the reaction mixture at temperature in the range of 21 to 29° C. for period in the range of 12-24 h;
iv. removing methanol from the reaction mixture as obtained from step (iii) under vacuum followed by adding sodium hydroxide solution to make the pH of the solution at 14.0±1.0;
v. extracting aqueous layer as obtained from step (iv) with dichloromethane followed by removing dichloromethane under vacuum to obtain white solid;
vi. reacting the white solid obtained from step (v) with 3,4,6-(R3,R2,R1-substituted) 5-chloromethyl salicylaldehyde in the molar ratio ranging between 1:3 to 1:5 in dry toluene under reflux at temperature ranging between 110 to 120° C. for period in the range of 8-12 h to get a white crystalline solid;
vii. Washing the white crystalline solid obtained from step (vi) with toluene and diethyl ether, dissolving the washed solid in dichloromethane, and washing the dichloromethane layer with aqueous sodium bicarbonate (10%);
viii. drying dichloromethane layer obtained from step (vii) over anhydrous sodium sulphate followed by filtration and removing dichloromethane from the filtrate gives white crystalline solid;
ix. treating the white crystalline solid obtained in step (viii) with a chiral 1,2-diamine in a molar ratio ranging between 1:1.5 to 1:3 in refluxed condition for period in the range, of 2-10 h at temperature in the range of 65 to 66° C. in presence of dry tetrahydrofuran;
x. evaporating tetrahydrofuran from the solution obtained from step (ix) under vacuum to get yellow solid after washing with methanol and diethyl ether;
xi. treating the yellow solid obtained from step (x) with reducing agent in a molar ratio ranging between 1:4 to 1:8 in methanol at room temperature in the range of 25 to 27° C. for period in the range of 1 to 3 hr;
xii. Evaporating methanol from the reaction mixture from step (xi), washing the solid obtained by dichloromethane and water to give chiral oligomeric [H₄]salen ligand of general formula 1.

In yet another embodiment of the present invention, linker used is selected from the group consisting of N-Boc piperazine, homopiperazine or 1,5-diazacane.

In yet another embodiment of the present invention, tertiary amine used is selected from the group consisting of triethylamine, triisopropylamine, N,N-diisopropylethylamine or 2,6-lutidine.

In yet another embodiment of the present invention, reagent used for the removal of t-butoxycarbonyl group from N-protected linker is selected from the group consisting of trifluoroacetic acid (TFA), paratoluenesulfonic acid (PTSA), anhydrous alkali metal carbonate selected from sodium carbonate, potassium carbonate, rubidium carbonate and cesium carbonate, metallic sodium and inorganic mineral acid like hydrochloric acid (HCl).

In yet another embodiment of the present invention, R₁, R₂, R₃ are selected from the group consisting of H, methyl, ethyl, propyl, isopropyl, n-butyl, isobutyl, t-butyl, or alkoxy selected from methoxy, ethoxy, butoxy or halogen selected from the group consisting of fluorine, chlorine, bromine and iodine.

In yet another embodiment of the present invention, chiral 1,2-diamine used is selected from the group consisting of (S)-1,2-diaminopropane, (R)-1,2-diaminopropane, (1R,2R)-(−)-1,2-diaminocyclohexane, (1S,2S)-(+)-1,2-diaminocyclohexane, (1R,2R)-(+)-1,2-diphenyl-1,2-diaminoethane, (1S,2S)-(−)-1,2-diphenyl-1,2-diaminoethane, (R)-(+)-1,1′-binaphthyl-2,2′-diamine and (S)-(−)-1,1′-binaphthyl-2,2′-diamine.

In yet another embodiment of the present invention, reducing agent used is selected from the group consisting of lithium aluminium hydride (LiAlH₄), sodium borohydride (NaBH₄), H₂/palladium-charcoal in an organic solvent selected from methanol, ethanol, n-propanol, iso-propanol, n-butanol, iso-butanol, tert-butanol, acetone, acetonitrile, propionitrile, toluene, xylene, diethylehter, tetrahydrofuran, dichloromethane and dichloroethane.

In yet another embodiment, present invention provides a process for preparation of nitroalcohol by asymmetric nitroaldol reactions using chiral homogeneous catalyst as claimed in claim 1 and the said process comprising the step of:
a) mixing 1 to 10 mol % of chiral ligand of general formula 1 in solvent;
b) adding the mixture as obtained in step (a) with metal salt in molar ratio ranging between 1:1 to 1:3 and with additive followed by stirring for period in the range of 1 to 3 h at a temperature ranging between 25-27° C. to generate in-situ active catalyst;
c) adding nitromethane and an aldehydes into the solution as obtained in step (b) and stirring the reaction mixture for a period ranging between 15 to 40 h preferably from 20-30 h at a temperature ranging between −20 to 110° C., preferably in the range of 0-60° C. more preferably from 10-30° C.;
d) evaporating solvent from the reaction mixture obtained from step (c) followed by repeatedly extracting by n-hexane and retrieving the solid by filtration/centrifugation;
e) evaporating the solvent from the combined filtrate as obtained in step (d) under vacuum to obtain crude nitroalcohol;
f) purifying the residue as obtained in step (e) by column chromatography using mixture of n-hexane:ethylacetate (90:10) to obtain 44 to 98% nitroaldol with 64-96% enantiomeric excess (ee).

In yet another embodiment of the present invention, the solvent used in step (a) is selected from the group consisting of aliphatic hydrocarbons such as n-hexane, n-heptane, n-octane, cyclohexane; halogenated hydrocarbons such as dichloromethane, dichloroethane and carbon tetrachloride; aromatic hydrocarbons such as benzene, toluene, xylene, mesitylene, chloronenzene, nitrobenzene; ethers such as tetrahydrofuran, diethylether, tert-butylmethyl ether, cyclopentylmethyl ether and dimethoxyethane; alcohols such as methanol, ethanol, n-propanol, iso-propanol, n-butanol, iso-butanol and tert-butanol; esters such as methyl acetate, ethyl acetate and butyl acetate; nitrites such as acetonitrile, and butyronitrile; amides such as N,N-dimethylformamide, N,N-dimethylacetamide and N-methylpyrrolidone; and ureas such as tetramethylurea or combination thereof.

In yet another embodiment of the present invention, the metal salt used in step (b) is selected from the group consisting of copper chloride, copper bromide, copper iodide, copper acetate, copper sulphate and copper triflate.

In yet another embodiment of the present invention, the additive used is selected from but not limiting to the group comprising of primary amine, secondary amine, tertiary amine, pyridine, 2-methyl pyridine, 2,6-lutidine, trimethylamine and triethylamine.

In yet another embodiment of the present invention, aldehydes used is selected from but not limiting to the group comprising of aromatic aldehyde, aliphatic aldehydes, α,β-unsaturated aldehydes and alicyclic aldehydes.

In yet another embodiment of the present invention, the chiral homogeneous catalyst used in step (a) ranges between 0.5 to 50.0 mol %, preferably in the range of 1.0 to 35.0 mol % more preferably in the range of 5.0-20.0 mol % based on aldehydes.

In yet another embodiment of the present invention, additive used in step (b) ranges between 1 to 40 mol %, preferably in the range of 1 to 10 mol % based on aldehyde.

In yet another embodiment of the present invention, nitromethane used in step (c) is ranging between 200 to 1200 mol % with respect to aldehydes used.

In yet another embodiment of the present invention, catalyst obtained in step (d) as solid is recyclable.

### DESCRIPTION OF THE INVENTION

Present invention provides chiral homogeneous catalyst for asymmetric nitroaldol reaction. The present invention also relates to the preparation of highly efficient recyclable chiral homogeneous catalysts, which are copper complexes of oligomeric [H₄]salen for their use in asymmetric nitroaldol reaction of various aromatic, aliphatic, α,β-unsaturated aldehydes and alicyclic aldehydes to provide different 1,2-nitro alcohols at room temperature, which are converted to pharmaceutically important molecules in few convenient steps in high yield and excellent enantioselectivity (>95%).

In a typical process for the synthesis of novel recyclable chiral homogeneous catalyst cynauric chloride was allowed to reflux with N-Boc piperazine in a molar ratio of 1:3 in dry tetrahydrofuran for a period of 12 to 18 h. After that the solvent was removed under vacuum and the solid obtained was dissolved in dichloromethane, washed with water and then dried over anhydrous sodium sulphate. After evaporation of dichloromethane under vacuum the solid obtained was re-crystallized from a mixture of dichloromethane and hexane. The white crystalline solid thus obtained was taken in methanol and 6(N) hydrochloric acid was added to the reaction mass at 0° C. The resulting reaction mixture was allowed to stir at the same temperature for 3 h and then at room temperature (21 to 29° C.) for a period of 12-16 h. The volatile organic component was removed under vacuum and 1(M) sodium hydroxide solution was added to it till the pH of the solution reached to 14. The aqueous layer was then extracted with dichloromethane (3 times) and the combined organic layer was collected and dried over anhydrous sodium sulphate. Removal of the dichloromethane under vacuum gave white crystalline solid which was taken in dry toluene to which a solution of 3/4/6-(R₃/R₂/R₁-substituted)-5-(chloromethyl)-2-hydroxybenzaldehyde in dry toluene was added drop-wise in a molar ratio of 1:3. The reaction mass was allowed to reflux for a period of 8-12 h. After completion of the reaction solid obtained was filtered and washed successively with toluene and solvent ether. The white solid obtained in this step was taken in dichloromethane and neutralized with aqueous saturated solution sodium bicarbonate. The organic layer was separated from which the solvent was removed under vacuum to give white crystalline solid, which was allowed to react with chiral 1,2-diaminocyclohexane in dry tetrahydrofuran for 2 h at room temperature. Afterwards tetrahydrofuran was removed under vacuum to give a bright yellow solid which was washed with methanol. The yellow thus solid obtained was taken in methanol and treated with sodium borohydride in a molar ratio of 1:4 and stirred for 2-4 h at room temperature. After completion of the reaction, the solvent was removed and the solid thus obtained was taken in dichloromethane and washed with water. Finally the removal of organic layer after drying over anhydrous Na₂SO₄ gave the reduced oligomeric salan ligand as a white solid.

Enantiomeric excess (ee) and optical purity of the products were determined by the use of programmable high-performance liquid chromatography system (HPLC, CLASS-VP 10A, 20 μL injection loop, PDA detector, Shimadzu), Gas chromatography (GC 2010 B, Shimadzu) and Autometic polarimeter (Digipol-781, Rudolph Instrument, USA). Perkin-Elmer Series II, 2400 CHN analyzer was used for the microanalysis of the samples. ¹H, ¹³C NMR Spectra were recorded on 200 and 500 MHz Spectrometer (Breker F113V), FTIR spectra were obtained using KBr (Perkin-Elmer spectrum GX spectrophotometer). 2-t-butyl-phenol, copper acetate monohydrate (Aldrich, USA), 1R,2R-(−)-1,2-diaminocyclohexane, (Floka, USA), paraformaldehyde, 2,6-dimethyl pyridine (Aldrich, USA), stanous chloride (Merk, Germany), hydrochloric acid (Ranboxy, India) were used as received. All the solvents used in the present study were dried by known purification technique. All chemical reaction were carried out under anhydrous conditions using nitrogen atmosphere and oven-dried glasswares unless otherwise stated.

The chiral recyclable homogeneous catalyst thus obtained was stirred with an appropriate aldehyde in an appropriate solvent containing an appropriate additive and an appropriate nitroalkane for 25 h at 25-27° C. The completion of the reaction was monitored by thin layer chromatography (TLC). The reaction mass was extracted from the catalyst by hexane and was dried over magnesium sulphate then evaporated under vacuum. The residue was purified by column chromatography by using n-hexane:ethylacetate (90:10) to give chiral nitroaldol product in high enantiomeric excess as determined by HPLC analysis using chiral column OD, OD-H and AD.

The present invention relates to the preparation of chiral compounds especially chiral nitro alcohols suitable for various applications. These chiral nitro alcohols were synthesized by asymmetric nitroaldol reaction of aldehydes and nitroalkane using recyclable homogenous chiral copper complex as catalyst in the presence of an organic base as an additive at room temperature. The inventive steps adopted in the present invention is to design catalyst is such that the chiral induction in the present asymmetric catalysis of the aldehydes gives very good to excellent yields and enentiomeric excess (ees) of the product nitroaldol at low catalyst loading at room temperature along with easy catalyst separation and recyclability.

The novelty of the invention is that it is for the first time novel oligomeric [H₄]salen derived copper complex is used as recyclable catalyst for the asymmetric nitroaldol reaction of different aldehydes at low catalyst loading and room temperature in the presence of inexpensive organic base as an additive.

### EXAMPLES

Following examples are given by way of illustration and therefore should not be construed to limit the scope of the invention.

### Example 1

In a typical process for the preparation of asymmetric recyclable homogeneous catalyst described in following Steps:

### Step 1

### Tri-tert-butyl 4,4′,4″-(1,3,5-triazine-2,4,6-triyl)tris(piperazine-1-carboxylate)

In a 3 necked round-bottom flask, cyanuric chloride (10 mmol, 1.84 g) and 1-Boc piperazine (BPIP, 30 mmol, 5.59 g) was added together in 50 ml of tetrahydrofuran and stirred at room temperature 25° C. for 1 h. Diisopropylethylamine (100 mmol, 17.4 ml) was added to the reaction mixture and the resulting solution was allowed to stir for 1 h at room temperature 25° C. followed by heating to reflux at 85° C. for 16 h (checked on TLC, R_{f} 0.6 in 95:5 dichloromethane:methanol). After completion of reaction, the reaction mixture was allowed to cool at room temperature 27° C. and then the solvent was evaporated under vacuum. The solid thus obtained was dissolved in 100 ml of dichloromethane and washed successively with water (3×100 ml) and brine (2×100 ml). The organic layer was dried over anhydrous sodium sulphate, filtered and the solvent was evaporated under vacuum to yield light yellow solid which on recrystalization from dichloromethane-hexane gives white crystalline solid (5.96 g, 94%)

¹H NMR (CDCl₃, 500 MHz) δ: 3.73 (m, 12H), 3.44 (m, 12H), 1.48 (s, 27H); ¹³C NMR (CDCl₃, 125 MHz) δ: 167.2, 156.7, 81.7, 44.9, 30.3; HRMS 634.4069; Anal calcd for C₃₀H₅₁N₉O₆: C, 56.85; H, 8.11; N, 19.89. found: C, 56.92; H, 8.16; N, 19.85.

### Step 2

### 2,4,6-Tri(piperazin-1-yl)-1,3,5-triazine

5 g of white crystalline solid obtained in step (1) was taken in 50 ml of methanol in a round bottom flask. The temperature of the reaction mass was lowered to 0° C. followed by the addition of 30 ml of 6 (N) hydrochloric acid and the resulting solution was allowed to stir at same temperature for 3 h then at room temperature 25° C. for 12 h. After completion of the reaction (checked on TLC) the volatile organic solvent was removed from the reaction mixture under vacuum and 10% sodium hydroxide solution was added to it to make alkaline (pH 14). The milky white solution thus obtained was extracted with dichloromethane (3×50 ml) and the combined organic layer was dried over anhydrous sodium sulphate. After filtration, evaporation of the organic solvent under vacuum leads to the desired product as a white solid which was recrystallized from chloroform-hexane to obtain a white crystalline material (2.42 g, 92%).

¹H NMR (CDCl₃, 500 MHz) δ: 3.73 (m, 12H), 2.87 (m, 12H), 1.86 (s, 3H); ¹³C NMR (CDCl₃, 125 MHz) δ: 167.2, 47.9, 46.2; HRMS 334.2279; Anal calcd for C₃₀H₅₁N₉O₆: C, 54.03; H, 8.16; N, 37.81. found: C, 54.08; H, 8.12; N, 37.88.

### Step 3

### 5,5′,5″-((4,4′,4″-(1,3,5-Triazine-2,4,6-triyl)tris(piperazine-4,1-diyl))tris(methylene))tris(3-(tert-butyl)-2-hydroxybenzaldehyde)

White crystalline solid obtained in step 2 (2 g, 6 mmol) was taken in 50 ml of dry toluene and a solution of 3-tertiary-butyl-5-(chloromethyl)-2-hydroxybenzaldehyde (4.2 g, 18.4 mmol in 10 ml of dry toluene) was added to it. The resulting solution was refluxed at 110° C. for 8 h. The solid precipitated out was filtered and washed successively with toluene and diethyl ether. Afterwards the solid was taken in dichloromethane and neutralized with aqueous sodium bicarbonate solution. The organic phase was collected, dried over anhydrous sodium suphate followed by evaporation of the solvent leads to white crystalline solid (5.16 g, 95%, TLC, R_{f} 0.42 in 95:5 dichloromethane:methanol).

¹H NMR (CDCl₃, 500 MHz) δ: 11.72 (s, 3H), 9.87 (s, 3H), 7.46 (s, 3H), 7.37 (s, 3H), 3.76 (s, 12H), 3.49 (s, 6H), 2.43 (m, 12H), 1.42 (s, 27H); ¹³C NMR (CDCl₃, 125 MHz) δ:197.1, 166.3, 160.3, 138.1, 135.2, 132.0, 128.6, 120.4, 62.4, 53.0, 43.1, 34.8, 29.3; HRMS 904.1027; Anal calcd for C₅₁H₆₉N₉O₆: C, 67.75; H, 7.69; N, 13.84. found: C, 67.80; H, 7.71; N, 13.82.

### Step 4

A solution of (1R,2R)-(−)-1,2-diaminocyclohexane (3.2 mmol, 366 mg in 10 ml of tetrahydrofuran) was added drop wise to another solution of white crystalline solid obtained in step 3 (2 mmol, 1800 mg in 10 ml of tetrahydrofuran) and the resulting mixture was allowed to reflux at 85° C. for 2 h (checked on TLC). The resulting dark yellow solution was cooled to room temperature 26° C. followed by evaporation of solvent under vacuum. The yellow solid thus obtained was washed with methanol to get the desired product (2.0 g, 98%). Anal calcd for C₄₄H₆₂N₁₁O₂ (single unit): C, 68.01; H, 8.04; N, 19.83. found: C, 67.90; H, 7.92; N, 19.92. ¹H NMR (CDCl₃, 500 MHz) δ: 13.8-13.9 (m, 2H), 8.8-8.4 (m, 2H), 6.8-7.2 (m, 4H), 3.3-3.7 (m, 18H), 2.3-2.4 (m, 8H), 1.8-1.9 (m, 2H), 1.2-1.5 (m, 26H); ¹³C NMR (CDCl₃, 125 MHz) δ: 24.3, 29.4, 34.7, 43.0, 52.9, 62.8, 72.2, 72.5, 118.3, 126.1, 130.3, 130.4, 136.8, 159.3, 159.4, 165.2.

### Step 5

1 g of the yellow crystalline solid obtained in step 4 was dissolved in dry methanol:dichloromethane (4:1; 50 ml), then 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 900 mg; 90%) Anal calcd for C₄₄H₆₆N₁₁O₂ (single unit): C, 67.66; H, 8.52; N, 19.73. found: C, 67.52; H, 8.44; N, 19.80. [α]_{D}²⁰=+22.12 (C=1, CHCl₃).

### Example 2

### Step 1

A solution of (1S,2S)-(+)-1,2-diaminocyclohexane (3.2 mmol, 366 mg in 10 ml of tetrahydrofuran) was added drop wise to the solution of the product obtained in step 3 of example 1 (2 mmol, 1.8 g in 10 ml of tetrahydrofuran) and the resulting mixture was allowed to reflux at 85° C. for 2 h (checked on TLC). The resulting dark yellow solution was cooled to room temperature 29° C. followed by evaporation of solvent under vacuum. The yellow solid thus obtained was washed with methanol to get the desired product (2.0 g; 98%). Anal calcd for C₄₄H₆₂N₁₁O₂ (single unit): C, 68.01; H, 8.04; N, 19.83. found: C, 67.92; H, 7.91; N, 19.95. IR (KBr); v: 3378, 2946, 2807, 1957, 1626, 1536 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 21° C. for 10 h. After completion of the reaction, solvent was removed under vacuum and the solid obtained was taken in dichloromethane, washed with water and finally evaporation of the solvent gives the desired product as white solid. (Yield, 900 mg; 90%) Anal calcd for C₄₄H₆₆N₁₁O₂ (single unit): C, 67.66; H, 8.52; N, 19.73. found: C, 67.50; H, 8.40; N, 19.94. IR (KBr); v: 3301, 2931, 2858, 2807, 2363, 1540, 1480 cm⁻¹.

### Example 3

### Step 1

A solution of (1R,2R)-(+)-1,2-diphenylethylenediamine (3.2 mmol, 678 mg in 10 ml of tetrahydrofuran) was added drop wise to the solution of the product obtained in step 3 of example 1 (2 mmol, 1.8 g in 10 ml of tetrahydrofuran) and the resulting mixture was allowed to reflux at 85° C. for 4 h (checked on TLC). The resulting dark yellow solution was cooled to room temperature 29° C. followed by evaporation of solvent under vacuum. The yellow solid thus obtained was washed with methanol to get the desired product (Yield, 2.24 g; 96%). Anal calcd for C₅₂H₆₄N₁₁O₂ (single unit): C, 71.37; H, 7.37; N, 17.61. found: C, 71.19; H, 7.28; N, 17.75. IR(KBr); v: 3651, 3381, 2949, 1953, 1628, 1539 cm⁻¹.

¹H NMR (CDCl₃, 500 MHz) δ: 13.6-13.8 (m, 2H), 8.2-8.4 (m, 2H), 6.9-7.2 (m, 14H), 4.6-4.7 (m, 3H), 3.3-3.7 (m, 14H), 2.2-2.4 (m, 9H), 1.7 (m, 2H), 1.4-1.5 (m, 18H); ¹³C NMR (CDCl₃, 125 MHz) δ: 30.9, 36.2, 44.5, 54.4, 64.2, 81.6, 83.8, 119.7, 129.5, 129.8, 132, 132.2, 133.3, 140.5, 141.0, 141.8, 160.8, 166.7.

### Step 2

1 g of the yellow crystalline solid obtained in step 1 of this example was dissolved in dry methanol:dichloromethane (4:1; 50 ml), to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 895 mg; 90%). Anal calcd for C₅₂H₆₈N₁₁O₂ (single unit): C, 71.04; H, 7.80; N, 17.52. found: C, 71.20; H, 7.68; N, 17.70. IR(KBr); v: 3301, 2920, 2859, 2364, 1950, 1540, 1481, 1438 cm⁻¹. [α]_{D}²⁰=−29.9 (C=1, CHCl₃).

### Example 4

### Step 1

A solution of (1S,2S)-(−)-1,2-diphenylethylenediamine (3.2 mmol, 678 mg in 10 ml of tetrahydrofuran) was added drop wise to the solution of the product obtained in step 3 of example 1 (2 mmol, 1.8 g in 10 ml of tetrahydrofuran) and the resulting mixture was allowed to reflux at 85° C. for 4 h (checked on TLC). The resulting dark yellow solution was cooled to room temperature 26° C. followed by evaporation of solvent under vacuum. The yellow solid thus obtained was washed with methanol to get the desired product (Yield, 2.24 g; 96%). Anal calcd for C₅₂H₆₄N₁₁O₂ (single unit): C, 71.37; H, 7.37; N, 17.61. found: C, 71.22; H, 7.24; N, 17.72. IR(KBr); v: 3652, 3383, 2946, 1952, 1625, 1538 cm⁻¹.

### Step 2

1 g of the yellow crystalline solid obtained in step 1 of this example was dissolved in dry methanol:dichloromethane (4:1; 50 ml), then 4 equivalents of sodium borohydride was added to it in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 895 mg; 90%). Anal calcd for C₅₂H₆₈N₁₁O₂ (single unit): C, 71.04; H, 7.80; N, 17.52. found: C, 71.20; H, 7.66; N, 17.66. IR(KBr); v: 3302, 2922, 2858, 2368, 1955, 1545, 1471, 1434 cm⁻¹.

### Example 5

### Step 1

A solution of (R)-(+)-1,1′-binaphthyl-2,2′-diamine (3.2 mmol, 910 mg in 10 ml of tetrahydrofuran) was added drop wise to the solution of the product obtained in step 3 of example 1 (2 mmol, 1.8 g in 10 ml of tetrahydrofuran) and the resulting mixture was allowed to reflux at 85° C. for 8 h (checked on TLC). The resulting dark yellow solution was cooled to room temperature 24° C. followed by evaporation of solvent under vacuum. The yellow solid thus obtained was washed with methanol to get the desired product (Yield, 2.34 g; 92%). Anal calcd for C₅₈H₆₄N₁₁O₂ (single unit): C, 73.55; H, 6.81; N, 16.27. found: C, 73.34; H, 6.66; N, 16.42. IR (KBr); v: 3372, 2942, 2810, 1950, 1616, 1530 cm⁻¹.

### Step 2

1 g of the yellow crystalline solid obtained in step 1 of this example was dissolved in dry methanol:dichloromethane (4:1; 50 ml), then 4 equivalents of sodium borohydride was added to it in 4 equal portions and the reaction mixture was allowed to stir at room temperature 24° C. for 10 h. After completion of the reaction, solvent was removed under vacuum and the solid obtained was taken in dichloromethane, washed with water and finally evaporation of the solvent gives the desired product as white solid. (Yield, 901 mg; 90%). Anal calcd for C₅₈H₆₈N₁₁O₂ (single unit): C, 73.23; H, 7.21; N, 16.20. found: C, 73.34; H, 7.07; N, 16.38. IR (KBr); v: 3301, 2922, 2855, 1952, 1540, 1430 cm⁻¹. [α]_{D}²⁰=+17.52 (C=1, CHCl₃).

### Example 6

### Step 1

A solution of (S)-(−)-1,1′-binaphthyl-2,2′-diamine (3.2 mmol, 910 mg in 10 ml of tetrahydrofuran) was added drop wise to the solution of the product obtained in step 3 of example 1 (2 mmol, 1.8 g in 10 ml of tetrahydrofuran) and the resulting mixture was allowed to reflux at 85° C. for 8 h (checked on TLC). The resulting dark yellow solution was cooled to room temperature 26° C. followed by evaporation of solvent under vacuum. The yellow solid thus obtained was washed with methanol to get the desired product (Yield, 2.34 g; 92%). Anal calcd for C₅₈H₆₄N₁₁O₂ (single unit): C, 73.55; H, 6.81; N, 16.27. found: C, 73.39; H, 6.68; N, 16.40. IR (KBr); v: 3374, 2945, 2815, 1955, 1618, 1536 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 26° C. for 10 h. After completion of the reaction, solvent was removed under vacuum and the solid obtained was taken in dichloromethane, washed with water and finally evaporation of the solvent gives the desired product as white solid. (Yield, 901 mg; 90%). Anal calcd for C₅₈H₆₈N₁₁O₂ (single unit): C, 73.23; H, 7.21; N, 16.20. found: C, 73.35; H, 7.10; N, 16.37. IR (KBr); v: 3321, 2926, 2858, 1956, 1548, 1438 cm⁻¹.

### Example 7

### Step 1

A solution of (R)-1,2 diaminopropane (3.2 mmol, 238 mg in 10 ml of tetrahydrofuran) was added drop wise to the solution of the product obtained in step 3 of example 1 (2 mmol, 1.8 g in 10 ml of tetrahydrofuran) and the resulting mixture was allowed to reflux at 85° C. for 2 h (checked on TLC). The resulting dark yellow solution was cooled to room temperature 27° C. followed by evaporation of solvent under vacuum. The yellow solid thus obtained was washed with methanol to get the desired product (Yield, 1.85 g; 96%). Anal calcd for C₄₁H₅₈N₁₁O₂ (single unit): C, 66.82; H, 7.93; N, 20.91. found: C, 66.66; H, 7.85; N, 21.01. IR (KBr); v: 3328, 2935, 2824, 1945, 1625, 1556 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 27° C. for 10 h. After completion of the reaction, solvent was removed under vacuum and the solid obtained was taken in dichloromethane, washed with water and finally evaporation of the solvent gives the desired product as white solid. (Yield, 900 mg; 90%) Anal calcd for C₄₁H₆₂N₁₁O₂ (single unit): C, 66.46; H, 8.43; N, 20.79. found: C, 66.62; H, 8.25; N, 20.95. IR (KBr); v: 3334, 2938, 2856, 2803, 2383, 1578 cm⁻¹.

### Example 8

### Step 1

A solution of (S)-1,2-diaminopropane (3.2 mmol, 238 mg in 10 ml of tetrahydrofuran) was added drop wise to the solution of the product obtained in step 3 of example 1 (2 mmol, 1.8 g in 10 ml of tetrahydrofuran) and the resulting mixture was allowed to reflux at 85° C. for 2 h (checked on TLC). The resulting dark yellow solution was cooled to room temperature 27° C. followed by evaporation of solvent under vacuum. The yellow solid thus obtained was washed with methanol to get the desired product (Yield, 1.85 g; 96%). Anal calcd for C₄₁H₅₈N₁₁O₂ (single unit): C, 66.82; H, 7.93; N, 20.91. found: C, 66.62; H, 7.85; N, 21.03. IR (KBr); v: 3324, 2932, 2822, 1942, 1624, 1554 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 26° C. for 10 h. After completion of the reaction, solvent was removed under vacuum and the solid obtained was taken in dichloromethane, washed with water and finally evaporation of the solvent gives the desired product as white solid. (Yield 900 solid. (Yield, 900 mg; 90%) Anal calcd for C₄₁H₆₂N₁₁O₂ (single unit): C, 66.46; H, 8.43; N, 20.79. found: C, 66.63; H, 8.28; N, 20.92. IR (KBr); v: 3332, 2936, 2852, 2812, 2314, 1556 cm⁻¹.

### Example 9

### Step 1

### Tri-tert-butyl 4,4′,4″-(1,3,5-triazine-2,4,6-triyl)tris(1,4-diazepane-1-carboxylate)

In a 3 necked round-bottom flask, cyanuric chloride (10 mmol, 1.84 g) and 1-Boc homopiperazine (HPIP) (30 mmol, 6 g) was added together in 50 ml of tetrahydrofuran and the resulting solution was stirred at room temperature 26° C. for 1 h. Diisopropylethylamine (100 mmol, 17.4 ml) was added to the reaction mixture and the resulting solution was allowed to stir for 1 h at room temperature 26° C. followed by heating to reflux at 85° C. for 16 h (checked on TLC). After completion of reaction, the reaction mixture was allowed to cool at room temperature 26° C. and then the solvent was evaporated under vacuum. The solid thus obtained was dissolved in 100 ml of dichloromethane and washed successively with water (3×100 ml) and brine (2×100 ml). The organic layer was dried over anhydrous sodium sulphate, filtered and the solvent was evaporated under vacuum to yield light yellow solid which on recrystalization from dichloromethane-hexane gives white crystalline solid (Yield, 6.4 g; 95%) Anal calcd for C₃₃H₅₇N₉O₆: C, 58.64; H, 8.50; N, 18.65. found: C, 58.58; H, 8.41; N, 18.70.

### Step 2

### 2,4,6-tri(1,4-diazepan-1-yl)-1,3,5-triazine

5 g of white crystalline solid obtained in step (1) of this example was taken in 50 ml of methanol in a round bottom flask. The temperature of the reaction mass was lowered to 0° C. followed by the addition of 30 ml of 6 (N) hydrochloric acid and the resulting solution was allowed to stir at same temperature for 3 h then at room temperature 26° C. for 12 h. After completion of the reaction (checked on TLC) the volatile organic solvent was removed from the reaction mixture under vacuum and 10% sodium hydroxide solution was added to it to make alkaline (pH 14). The milky white solution thus obtained was extracted with dichloromethane (3×50 ml) and the combined organic layer was dried over anhydrous sodium sulphate. After filtration, evaporation of the organic solvent under vacuum leads to the desired product as a white solid which was recrystallized from chloroform-hexane to obtain a white crystalline material (Yield, 2.56 g; 92%). Anal calcd for C₁₈H₃₃N₉: C, 57.57; H, 8.86; N, 33.57. found: C, 57.66; H, 8.94; N, 33.47.

### Step 3

### 5,5′,5″-((4,4′,4″-(1,3,5-triazine-2,4,6-triyl)tris(1,4-diazepane-4,1diyl))tris(methylene))tris(3-(tert-butyl)-2-hydroxybenzaldehyde)

White crystalline solid obtained in step 2 of this example (2.25 g, 6 mmol) was taken in ml of dry toluene and a solution of 3-tertiary-butyl-5-(chloromethyl)-2-hydroxybenzaldehyde (4.2 g, 18.4 mmol in 10 ml of dry toluene) was added to it. The resulting solution was refluxed at 110° C. for 8 h. The solid precipitated out was filtered and washed successively with toluene and diethyl ether. Afterwards the solid was taken in dichloromethane and neutralized with aqueous sodium bicarbonate solution. The organic phase was collected, dried over anhydrous sodium suphate followed by evaporation of the solvent leads to white crystalline solid (Yield, 5.45 g; 96%). Anal calcd for C₅₄H₇₅N₉O₆: C, 68.54; H, 7.99; N, 13.32. found: C, 68.60; H, 8.03; N, 13.24.

### Step 4

A solution of (1R,2R)-(−)-1,2-diaminocyclohexane (3.2 mmol, 366 mg in 10 ml of tetrahydrofuran) was added drop wise to another solution of white crystalline solid obtained in step 3 (2 mmol, 1890 mg in 10 ml of tetrahydrofuran) and the resulting mixture was allowed to reflux at 85° C. for 2 h (checked on TLC). The resulting dark yellow solution was cooled to room temperature 26° C. followed by evaporation of solvent under vacuum. The yellow solid thus obtained was washed with methanol to get the desired product (Yield, 2.04 g; 96%). Anal calcd for C₄₇H₆₈N₁₁O₂ (single unit): C, 68.92; H, 8.37; N, 18.81. found: C, 69.10; H, 8.45; N, 18.70. IR (KBr); v: 3376, 2942, 2803, 1952, 1622, 1531 cm⁻¹.

### Step 5

1 g of the yellow crystalline solid obtained in step 4 of this example was dissolved in dry methanol:dichloromethane (4:1; 50 ml) to which 4 equivalents of sodium borohydride was added in 4 equal portions and the reaction′ mixture was allowed to stir at room temperature 26° C. for 10 h. After completion of the reaction, solvent was removed under vacuum and the solid obtained was taken in dichloromethane, washed with water and finally evaporation of the solvent gives the desired product as white solid. (Yield, 913; mg, 91%) Anal calcd for C₄₇H₇₂N₁₁O₂ (single unit): C, 68.58; H, 8.82; N, 18.72. found: C, 68.72; H, 8.95; N, 18.60. IR (KBr); v: 3306, 2934, 2854, 2801, 2368, 1530, 1445 cm⁻¹.

### Example 10

### Step 1

A solution of (1S,2S)-(+)-1,2-diaminocyclohexane (3.2 mmol, 366 mg in 10 ml of tetrahydrofuran) was added drop wise to the solution of the product obtained in step 3 of example 9 (2 mmol, 1890 mg in 10 ml of tetrahydrofuran) and the resulting mixture was allowed to reflux at 85° C. for 2 h (checked on TLC). The resulting dark yellow solution was cooled to room temperature 25° C. followed by evaporation of solvent under vacuum. The yellow solid thus obtained was washed with methanol to get the desired product (Yield, 2.04 g; 96%). Anal calcd for C₄₇H₆₈N₁₁O₂ (single unit): C, 68.92; H, 8.37; N, 18.81. found: C, 69.08; H, 8.46; N, 18.66. IR (KBr); v: 3374, 2941, 2805, 1950, 1620, 1538 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 911 mg; 91%) Anal calcd for C₄₇H₇₂N₁₁O₂ (single unit): C, 68.58; H, 8.82; N, 18.72. found: C, 68.68; H, 8.94; N, 18.61. IR (KBr); v: 3302, 2930, 2850, 2841, 2328, 1535, 1441 cm⁻¹.

### Example 11

### Step 1

A solution of (1R,2R)-(+)-1,2-diphenylethylenediamine (3.2 mmol, 678 mg in 10 ml of tetrahydrofuran) was added drop wise to the solution of the product obtained in step 3 of example 9 (2 mmol, 1890 mg in 10 ml of tetrahydrofuran) and the resulting mixture was allowed to reflux at 85° C. for 4 h (checked on TLC). The resulting dark yellow solution was cooled to room temperature 25° C. followed by evaporation of solvent under vacuum. The yellow solid thus obtained was washed with methanol to get the desired product (Yield, 2.3 g; 95%). Anal calcd for C₅₅H₇₀N₁₁O₂ (single unit): C, 72.02; H, 7.69; N, 16.80. found: C, 72.20; H, 7.90; N, 16.65. IR(KBr); v: 3654, 3385, 2944, 1950, 1620, 1532 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 901 mg; 90%) Anal calcd for C₅₅H₇₄N₁₁O₂ (single unit): C, 71.71; H, 8.10; N, 16.72. found: C, 71.85; H, 8.22; N, 16.58. IR(KBr); v: 3304, 2926, 2858, 2360, 1955, 1545, 1483, 1432 cm⁻¹.

### Example 12

### Step 1

A solution of (1S,2S)-(−)-1,2-diphenylethylenediamine (3.2 mmol, 678 mg in 10 ml of tetrahydrofuran) was added drop wise to the solution of the product obtained in step 3 of example 9 (2 mmol, 1890 mg in 10 ml of tetrahydrofuran) and the resulting mixture was allowed to reflux at 85° C. for 4 h (checked on TLC). The resulting dark yellow solution was cooled to room temperature 25° C. followed by evaporation of solvent under vacuum. The yellow solid thus obtained was washed with methanol to get the desired product (Yield, 2.3 g; 95%). Anal calcd for C₅₅H₇₀N₁₁O₂ (single unit): C, 72.02; H, 7.69; N, 16.80. found: C, 72.21; H, 7.88; N, 16.65. IR(KBr); v: 3654, 3386, 2942, 1950, 1622, 1531 cm⁻¹.

### Step 2

1 g of the yellow crystalline solid obtained in step 1 of this example was dissolved in dry methanol:dichloromethane (4:1; 50 ml), then 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 895 mg; 90%) Anal calcd for C₅₅H₇₄N₁₁O₂ (single unit): C, 71.71; H, 8.10; N, 16.72. found: C, 71.88; H, 8.21; N, 16.63. IR(KBr); v: 3308, 2922, 2850, 2360, 1959, 1530, 1478, 1498 cm⁻¹.

### Example 13

### Step 1

A solution of (R)-(+)-1,1′-binaphthyl-2,2′-diamine (3.2 mmol, 910 mg in 10 ml of tetrahydrofuran) was added drop wise to the solution of the product obtained in step 3 of example 9 (2 mmol, 1890 mg in 10 ml of tetrahydrofuran) and the resulting mixture was allowed to reflux at 85° C. for 8 h (checked on TLC). The resulting dark yellow solution was cooled to room temperature 25° C. followed by evaporation of solvent under vacuum. The yellow solid thus obtained was washed with methanol to get the desired product (Yield, 2.5 g; 95%). Anal calcd for C₆₁H₇₀N₁₁O₂ (single unit): C, 74.06; H, 7.13; N, 15.57. found: C, 74.21; H, 7.22; N, 15.41. IR (KBr); v: 3365, 2989, 2834, 1935, 1666, 1578 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 890 mg; 89%) Anal calcd for C₆₁H₇₄N₁₁O₂ (single unit): C, 73.76; H, 7.51; N, 15.51. found: C, 73.91; H, 7.58; N, 15.42. IR (KBr); v: 3345, 2912, 2825, 1949, 1578, 1472 cm⁻¹.

### Example 14

### Step 1

A solution of (S)-(−)-1,1′-binaphthyl-2,2′-diamine (3.2 mmol, 910 mg in 10 ml of tetrahydrofuran) was added drop wise to the solution of the product obtained in step 3 of example 9 (2 mmol, 1890 mg in 10 ml of tetrahydrofuran) and the resulting mixture was allowed to reflux at 85° C. for 8 h (checked on TLC). The resulting dark yellow solution was cooled to room temperature 25° C. followed by evaporation of solvent under vacuum. The yellow solid thus obtained was washed with methanol to get the desired product (Yield, 2.5 g; 95%). Anal calcd for C₆₁H₇₀N₁₁O₂ (single unit): C, 74.06; H, 7.13; N, 15.57. found: C, 74.20; H, 7.22; N, 15.35. IR (KBr); v: 3323, 2967, 2878, 1959, 1617, 1538 cm⁻¹.

### Step 2

1 g of the yellow crystalline solid obtained in step 1 of this example was dissolved in dry methanol:dichloromethane (4:1, 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 901 mg; 90%) Anal calcd for C₆₁H₇₄N₁₁O₂ (single unit): C, 73.76; H, 7.51; N, 15.51. found: C, 73.95; H, 7.65; N, 15.40. IR (KBr); v: 3341, 2982, 2865, 1989, 1589 cm⁻¹.

### Example 15

### Step 1

A solution of (R)-1,2 diaminopropane (3.2 mmol, 238 mg in 10 ml of tetrahydrofuran) was added drop wise to the solution of the product obtained in step 3 of example 9 (2 mmol, 1890 mg in 10 ml of tetrahydrofuran) and the resulting mixture was allowed to reflux at 85° C. for 2 h (checked on TLC). The resulting dark yellow solution was cooled to room temperature 25° C. followed by evaporation of solvent under vacuum. The yellow solid thus obtained was washed with methanol to get the desired product (Yield, 1.9 g; 95%). Anal calcd for C₄₄H₆₄N₁₁O₂ (single unit): C, 67.84; H, 8.28; N, 19.78. found: C, 68.00; H, 8.42; N, 19.62. IR (KBr); v: 3314, 2922, 2812, 1942, 1694, 1544 cm⁻¹.

### Step 2

1 g of the yellow crystalline solid obtained in step 1 of this example was dissolved in dry methanol:dichloromethane (4:1; 50 ml) to which 4 equivalents of sodium borohydride was added to it in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 901 mg; 90%) Anal calcd for C₄₄H₆₈N₁₁O₂ (single unit): C, 67.49; H, 8.75; N, 19.68. found: C, 67.70; H, 8.82; N, 19.62. IR (KBr); v: 3338, 2926, 2842, 2882, 2324, 1559 cm⁻¹.

### Example 16

### Step 1

A solution of (S)-1,2 diaminopropane (3.2 mmol, 238 mg in 10 ml of tetrahydrofuran) was added drop wise to the solution of the product obtained in step 3 of example 9 (2 mmol, 1890 mg in 10 ml of tetrahydrofuran) and the resulting mixture was allowed to reflux at 85° C. for 2 h (checked on TLC). The resulting dark yellow solution was cooled to room temperature 25° C. followed by evaporation of solvent under vacuum. The yellow solid thus obtained was washed with methanol to get the desired product (Yield, 1.9 g; 95%). Anal calcd for C₄₄H₆₄N₁₁O₂ (single unit): C, 67.84; H, 8.28; N, 19.78. found: C, 68.02; H, 8.42; N, 19.65. IR (KBr); v: 3316, 2920, 2818, 1940, 1694, 1584 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 901 mg; 90%) Anal calcd for C₄₄H₆₈N₁₁O₂ (single unit): C, 67.49; H, 8.75; N, 19.68. found: C, 67.67; H, 8.80; N, 19.49. IR (KBr); v: 3328, 2920, 2848, 2880, 2328, 1550 cm⁻¹.

### Example 17

### Step 1

### tri-tert-butyl 5,5′,5″-(1,3,5-triazine-2,4,6-triyl)tris(1,5-diazocane-1-carboxylate)

In a 3 necked round-bottom flask, cyanuric chloride (10 mmol, 1.84 g) and 1-Boc tert-butyl 1,5-diazocane-1-carboxylate (30 mmol, 6.43 g) was added together in 50 ml of tetrahydrofuran and the resulting solution was stirred at room temperature 25° C. for 1 h. Diisopropylethylamine (100 mmol, 17.4 ml) was added to the reaction mixture and the resulting solution was allowed to stir for 1 h at room temperature 25° C. followed by heating to reflux at 85° C. for 16 h (checked on TLC). After completion of reaction, the reaction mixture was allowed to cool at room temperature 25° C. and then the solvent was evaporated under vacuum. The solid thus obtained was dissolved in 100 ml of dichloromethane and washed successively with water (3×100 ml) and brine (2×100 ml). The organic layer was dried over anhydrous sodium sulphate, filtered and the solvent was evaporated under vacuum to yield light yellow solid which on recrystalization from dichloromethane-hexane gives white crystalline solid (Yield, 6.68 g; 93%) Anal calcd for C₃₆H₆₃N₉O₆: C, 60.23; H, 8.84; N, 17.56. found: C, 60.31; H, 8.90; N, 17.50.

### Step 2

### 2,4,6-tri(1,5-diazocan-1-yl)-1,3,5-triazine

5 g of white crystalline solid obtained in step 1 of this example was taken in 50 ml of methanol in a round bottom flask. The temperature of the reaction mass was lowered to 0° C. followed by the addition of 30 ml of 6 (N) hydrochloric acid and the resulting solution was allowed to stir at same temperature for 3 h then at room temperature for 12 h. After completion of the reaction (checked on TLC) the volatile organic solvent was removed from the reaction mixture under vacuum and 10% sodium hydroxide solution was added to it to make alkaline (pH 14). The milky white solution thus obtained was extracted with dichloromethane (3×50 ml) and the combined organic layer was dried over anhydrous sodium sulphate. After filtration, evaporation of the organic solvent under vacuum leads to the desired product as a white solid which was recrystallized from chloroform-hexane to obtain a white crystalline material (Yield, 2.73 g; 94%). Anal calcd for C₂₁H₃₉N₉: C, 60.40; H, 9.41; N, 30.19. found: C, 60.45; H, 9.43; N, 30.09.

### Step 3

### 5,5′,5″-((5,5′,5″-(1,3,5-triazine-2,4,6-triyl)tris(1,5-diazocane-5,1-diyl))tris(methylene))tris(3-(tert-butyl)-2-hydroxybenzaldehyde)

White crystalline solid obtained in step 2 of this example (2.5 g, 6 mmol) was taken in ml of dry toluene and a solution of 3-tertiary-butyl-5-(chloromethyl)-2-hydroxybenzaldehyde (4.2 g, 18.4 mmol in 10 ml of dry toluene) was added to it. The resulting solution was refluxed at 110° C. for 8 h. The solid precipitated out was filtered and washed successively with toluene and diethyl ether. Afterwards the solid was taken in dichloromethane and neutralized with aqueous sodium bicarbonate solution. The organic phase was collected, dried over anhydrous sodium suphate followed by evaporation of the solvent leads to white crystalline solid (Yield, 5.46 g; 92%). Anal calcd for C₅₇H₈₁N₉O₆: C, 69.27; H, 8.26; N, 12.76. found: C, 69.34; H, 8.30; N, 12.71.

### Step 4

A solution of (1R,2R)-(−)-1,2-diaminocyclohexane (3.2 mmol, 366 mg in 10 ml of tetrahydrofuran) was added drop wise to another solution of white crystalline solid obtained in step 3 of this example (2 mmol, 1976 mg in 10 ml of tetrahydrofuran) and the resulting mixture was allowed to reflux at 85° C. for 2 h (checked on TLC). The resulting dark yellow solution was cooled to room temperature 25° C. followed by evaporation of solvent under vacuum. The yellow solid thus obtained was washed with methanol to get the desired product (Yield, 2.12 g; 96%). Anal calcd for C₅₀H₇₄N₁₁O₂ (single unit): C, 69.73; H, 8.66; N, 17.89. found: C, 69.88; H, 8.75; N, 17.71. IR (KBr); v: 3374, 2940, 2801, 1950, 1629, 1531 cm⁻¹.

### Step 5

1 g of the yellow crystalline solid obtained in step 4 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 911; mg, 91%) Anal calcd for C₅₀H₇₈N₁₁O₂ (single unit): C, 69.41; H, 9.09; N, 17.81. found: C, 69.61; H, 9.19; N, 17.66. IR (KBr); v: 3361, 2901, 2808, 2856, 2303, 1510, 1446 cm⁻¹.

### Example 18

### Step 1

A solution of (1S,2S)-(−)-1,2-diaminocyclohexane (3.2 mmol, 366 mg in 10 ml of tetrahydrofuran) was added drop wise to another solution of white crystalline solid obtained in step 3 of example 17 (2 mmol, 1976 mg in 10 ml of tetrahydrofuran) and the resulting mixture was allowed to reflux at 85° C. for 2 h (checked on TLC). The resulting dark yellow solution was cooled to room temperature 25° C. followed by evaporation of solvent under vacuum. The yellow solid thus obtained was washed with methanol to get the desired product (Yield, 2.12 g; 96%). Anal calcd for C₅₀H₇₄N₁₁O₂ (single unit): C, 69.73; H, 8.66; N, 17.89. found: C, 69.86; H, 8.75; N, 17.70. IR (KBr); v: 3328, 2926, 2878, 1967, 1623, 1565 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 911; mg, 91%) Anal calcd for C₅₀H₇₈N₁₁O₂ (single unit): C, 69.41; H, 9.09; N, 17.81. found: C, 69.65; H, 9.17; N, 17.63. IR (KBr); v: 3311, 2938, 2867, 2847, 2367, 1545, 1476 cm⁻¹.

### Example 19

### Step 1

A solution of (1R,2R)-(+)-1,2-diphenylethylenediamine (3.2 mmol, 678 mg in 10 ml of tetrahydrofuran) was added drop wise to another solution of white crystalline solid obtained in step 3 of example 17 (2 mmol, 1976 mg in 10 ml of tetrahydrofuran) and the resulting mixture was allowed to reflux at 85° C. for 4 h (checked on TLC). The resulting dark yellow solution was cooled to room temperature 25° C. followed by evaporation of solvent under vacuum. The yellow solid thus obtained was washed with methanol to get the desired product (Yield, 2.33 g; 93%). Anal calcd for C₅₈H₇₆N₁₁O₂ (single unit): C, 72.62; H, 7.99; N, 16.06. found: C, 72.85; H, 8.09; N, 15.90. IR(KBr); v: 3658, 3388, 2942, 1950, 1626, 1530 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 917; mg, 92%) Anal calcd for C₅₈H₈₀N₁₁O₂ (single unit): C, 72.31; H, 8.37; N, 15.99. found: C, 72.51; H, 8.48; N, 15.81. IR(KBr); v: 3351, 2929, 2839, 2327, 1958, 1567, 1434, 1487 cm⁻¹.

### Example 20

### Step 1

A solution of (1S,2S)-(−)-1,2-diphenylethylenediamine (3.2 mmol, 678 mg in 10 ml of tetrahydrofuran) was added drop wise to another solution of white crystalline solid obtained in step 3 of example 17 (2 mmol, 1976 mg in 10 ml of tetrahydrofuran) and the resulting mixture was allowed to reflux at 85° C. for 4 h. (checked on TLC). The resulting dark yellow solution was cooled to room temperature 25° C. followed by evaporation of solvent under vacuum. The yellow solid thus obtained was washed with methanol to get the desired product (Yield, 2.33 g; 93%). Anal calcd for C₅₈H₇₆N₁₁O₂ (single unit): C, 72.62; H, 7.99; N, 16.06. found: C, 72.83; H, 8.08; N, 15.90. IR(KBr); v: 3651, 3367, 2934, 1959, 1628, 1545 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 917; mg, 92%) Anal calcd for C₅₈H₈₀N₁₁O₂ (single unit): C, 72.31; H, 8.37; N, 15.99. found: C, 72.53; H, 8.49; N, 15.81. IR(KBr); v: 3391, 2929, 2839, 2369, 1998, 1565 cm⁻¹.

### Example 21

### Step 1

A solution of (R)-(+)-1,1′-binaphthyl-2,2′-diamine (3.2 mmol, 910 mg in 10 ml of tetrahydrofuran) was added drop wise to another solution of white crystalline solid obtained in step 3 of example 17 (2 mmol, 1976 mg in 10 ml of tetrahydrofuran) and the resulting mixture was allowed to reflux at 85° C. for 8 h (checked on TLC). The resulting dark yellow solution was cooled to room temperature followed by evaporation of solvent under vacuum. The yellow solid thus obtained was washed with methanol to get the desired product (Yield, 2.6 g; 92%). Anal calcd for C₆₄H₇₆N₁₁O₂ (single unit): C, 74.53; H, 7.43; N, 14.94. found: C, 74.71; H, 7.52; N, 14.77. IR (KBr); v: 3362, 2967, 2834, 1989, 1623, 1598 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 901; mg, 90%) Anal calcd for C₆₄H₈₀N₁₁O₂ (single unit): C, 74.24; H, 7.79; N, 14.88. found: C, 74.41; H, 7.91; N, 14.66. IR (KBr); v: 3341, 2956, 2854, 1926, 15987, 1454 cm⁻¹.

### Example 22

### Step 1

A solution of (S)-(−)-1,1′-binaphthyl-2,2′-diamine (3.2 mmol, 910 mg in 10 ml of tetrahydrofuran) was added drop wise to another solution of white crystalline solid obtained in step 3 of example 17 (2 mmol, 1976 mg in 10 ml of tetrahydrofuran) and the resulting mixture was allowed to reflux at 85° C. for 8 h (checked on TLC). The resulting dark yellow solution was cooled to room temperature 25° C. followed by evaporation of solvent under vacuum. The yellow solid thus obtained was washed with methanol to get the desired product (Yield, 2.6 g; 92%). Anal calcd for C₆₄H₇₆N₁₁O₂ (single unit): C, 74.53; H, 7.43; N, 14.94. found: C, 74.70; H, 7.52; N, 14.76. IR (KBr); v: 3378, 2967, 2846, 1965, 1664, 1598 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 901; mg, 90%) Anal calcd for C₆₄H₈₀N₁₁O₂ (single unit): C, 74.24; H, 7.79; N, 14.88. found: C, 74.36; H, 7.85; N, 14.70. IR (KBr); v: 3376, 2929, 2854, 1934, 1575, 1443 cm⁻¹.

### Example 23

### Step 1

A solution of (R)-1,2-diaminopropane (3.2 mmol, 238 mg in 10 ml of tetrahydrofuran) was added drop wise to another solution of white crystalline solid obtained in step 3 of example 17 (2 mmol, 1976 mg in 10 ml of tetrahydrofuran) and the resulting mixture was allowed to reflux at 85° C. for 2 h (checked on TLC). The resulting dark yellow solution was cooled to room temperature 25° C. followed by evaporation of solvent under vacuum. The yellow solid thus obtained was washed with methanol to get the desired product (Yield, 2.0 g; 96%). Anal calcd for C₄₇H₇₀N₁₁O₂ (single unit): C, 68.75; H, 8.59; N, 18.76. found: C, 68.92; H, 8.65; N, 18.61. IR (KBr); v: 3320, 2934, 2820, 1941, 1623, 1553 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 901; mg, 90%) Anal calcd for C₄₇H₇₄N₁₁O₂ (single unit): C, 68.41; H, 9.04; N, 18.67. found: C, 68.56; H, 9.15; N, 18.50. IR (KBr); v: 3332, 2934, 2859, 2845, 2356, 1576 cm⁻¹.

### Example 24

### Step 1

A solution of (S)-1,2-diaminopropane (3.2 mmol, 238 mg in 10 ml of tetrahydrofuran) was added drop wise to another solution of white crystalline solid obtained in step 3 of example 17 (2 mmol, 1976 mg in 10 ml of tetrahydrofuran) and the resulting mixture was allowed to reflux at 85° C. for 2 h (checked on TLC). The resulting dark yellow solution was cooled to room temperature 25° C. followed by evaporation of solvent under vacuum. The yellow solid thus obtained was washed with methanol to get the desired product (Yield, 2.0 g; 96%). Anal calcd for C₄₇H₇₀N₁₁O₂ (single unit): C, 68.75; H, 8.59; N, 18.76. found: C, 68.90; H, 8.66; N, 18.64. IR (KBr); v: 3326, 2932, 2820, 1947, 1623, 1550 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 901; mg, 90%) Anal calcd for C₄₇H₇₄N₁₁O₂ (single unit): C, 68.41; H, 9.04; N, 18.67. found: C, 68.58; H, 9.12; N, 18.51. IR (KBr); v: 3330, 2936, 2853, 2808, 2380, 1575 cm⁻¹.

### Example 25

### Step 1

### 5,5′,5″-((4,4′,4″-(1,3,5-triazine-2,4,6-triyl)tris(piperazine-4,1-diyl))tris(methylene))tris(2-hydroxybenzaldehyde)

White crystalline solid obtained in step 2 of example 1 (2 g, 6 mmol) was taken in 50 ml of dry toluene and a solution of 5-(chloromethyl)-2-hydroxybenzaldehyde (3.13 g, 18.4 mmol in 10 ml of dry toluene) was added to it. The resulting solution was refluxed at 110° C. for 8 h. The solid precipitated out was filtered and washed successively with toluene and diethyl ether. Afterwards the solid, was taken in dichloromethane and neutralized with aqueous sodium bicarbonate solution. The organic phase was collected, dried over anhydrous sodium suphate followed by evaporation of the solvent leads to white crystalline solid (Yield, 4.2 g; 95%). Anal calcd for C₃₉H₄₅N₉O₆: C, 63.66; H, 6.16; N, 17.13. found: C, 63.72; H, 6.20; N, 17.08.

### Step 2

A solution of (1R,2R)-(−)-1,2-diaminocyclohexane (3.2 mmol, 366 mg in 10 ml of tetrahydrofuran) was added drop wise to another solution of white crystalline solid obtained in step 1 of this example (2 mmol, 1470 mg in 10 ml of tetrahydrofuran) and the resulting mixture was allowed to reflux at 85° C. for 2 h (checked on TLC). The resulting dark yellow solution was cooled to room temperature 25° C. followed by evaporation of solvent under vacuum. The yellow solid thus obtained was washed with methanol to get the desired product (Yield, 1.65 g; 97%). Anal calcd for C₃₆H₄₆N₁₁O₂ (single unit): C, 65.04; H, 6.97; N, 23.18. found: C, 65.22; H, 7.11; N, 22.99. IR (KBr); v: 3375, 2942, 2808, 1953, 1620, 1533 cm⁻¹.

### Step 3

1 g of the yellow crystalline solid obtained in step 2 was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 900 mg; 90%) Anal calcd for C₃₆H₅₀N₁₁O₂ (single unit): C, 64.65; H, 7.53; N, 23.04. found: C, 64.79; H, 7.62; N, 22.92. IR (KBr); v: 3311, 2939, 2855, 2802, 2360, 1546, 1487 cm⁻¹.

### Example 26

### Step 1

A solution of (1S,2S)-(+)-1,2-diaminocyclohexane (3.2 mmol, 366 mg in 10 ml of tetrahydrofuran) was added drop wise to another solution of white crystalline solid obtained in step 2 of example 25 (2 mmol, 1470 mg in 10 ml of tetrahydrofuran) and the resulting mixture was allowed to reflux at 85° C. for 2 h (checked on TLC). The resulting dark yellow solution was cooled to room temperature 25° C. followed by evaporation of solvent under vacuum. The yellow solid thus obtained was washed with methanol to get the desired product (Yield, 1.65 g; 97%). Anal calcd for C₃₆H₄₆N₁₁O₂ (single unit): C, 65.04; H, 6.97; N, 23.18. found: C, 65.21; H, 7.15; N, 23.02. IR (KBr); v: 3377, 2948, 2827, 1958, 1636, 1538 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 900 mg; 90%) Anal calcd for C₃₆H₅₀N₁₁O₂ (single unit): C, 64.65; H, 7.53; N, 23.04. found: C, 64.78; H, 7.62; N, 22.95. IR (KBr); v: 3381, 2938, 2867, 2856, 2346, 1545, 1456 cm⁻¹.

### Example 27

### Step 1

A solution of (1R,2R)-(+)-1,2-diphenylethylenediamine (3.2 mmol, 678 mg in 10 ml of tetrahydrofuran) was added drop wise to another solution of white crystalline solid obtained in step 1 of example 25 (2 mmol, 1470 mg in 10 ml of tetrahydrofuran) and the resulting mixture was allowed to reflux at 85° C. for 4 h (checked on TLC). The resulting dark yellow solution was cooled to room temperature 25° C. followed by evaporation of solvent under vacuum. The yellow solid thus obtained was washed with methanol to get the desired product (Yield, 1.94 g; 97%). Anal calcd for C₄₄H₄₈N₁₁O₂ (single unit): C, 69.27; H, 6.34; N, 20.20. found: C, 69.42; H, 6.48; N, 19.98. IR(KBr); v: 3658, 3386, 2944, 1953, 1627, 1538 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 904 mg; 90%) Anal calcd for C₄₄H₅₂N₁₁O₂ (single unit): C, 68.90; H, 6.83; N, 20.09. found: C, 68.72; H, 6.98; N, 19.98. IR(KBr); v: 3361, 2928, 2857, 2360, 1945, 1556, 1487, 1445 cm⁻¹.

### Example 28

### Step 1

A solution of (1S,2S)-(−)-1,2-diphenylethylenediamine (3.2 mmol, 678 mg in 10 ml of tetrahydrofuran) was added drop wise to another solution of white crystalline solid obtained in step 1 of example 25 (2 mmol, 1470 mg in 10 ml of tetrahydrofuran) and the resulting mixture was allowed to reflux at 85° C. for 4 h (checked on TLC). The resulting dark yellow solution was cooled to room temperature 25° C. followed by evaporation of solvent under vacuum. The yellow solid thus obtained was washed with methanol to get the desired product (Yield, 1.94 g; 97%). Anal calcd for C₄₄H₄₈N₁₁O₂ (single unit): C, 69.27; H, 6.34; N, 20.20. found: C, 69.45; H, 6.48; N, 20.03. IR(KBr); v: 3655, 3388, 2942, 1952, 1627, 1530 cm⁻¹.

### Step 2

1 g of the yellow crystalline solid obtained in step 1 of this example was dissolved in dry methanol:dichloromethane (4:1; 50 ml) to which 4 equivalents of sodium borohydride was added to it in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 904 mg; 90%). Anal calcd for C₄₄H₅₂N₁₁O₂ (single unit): C, 68.90; H, 6.83; N, 20.09. found: C, 68.75; H, 6.84; N, 19.98. IR(KBr); v: 3331, 2928, 2852, 2360, 1959, 1547, 1486, 1431 cm⁻¹.

### Example 29

### Step 1

A solution of (R)-(+)-1,1′-binaphthyl-2,2′-diamine (3.2 mmol, 910 mg in 10 ml of tetrahydrofuran) was added drop wise to another solution of white crystalline solid obtained in step 1 of example 25 (2 mmol, 1470 mg in 10 ml of tetrahydrofuran) and the resulting mixture was allowed to reflux at 85° C. for 8 h (checked on TLC). The resulting dark yellow solution was cooled to room temperature 25° C. followed by evaporation of solvent under vacuum. The yellow solid thus obtained was washed with methanol to get the desired product (Yield, 2.1 g; 95%). Anal calcd for C₅₀H₄₈N₁₁O₂ (single unit): C, 71.92; H, 5.79; N, 18.45. found: C, 72.08; H, 5.87; N, 18.22. IR (KBr); v: 3377, 2948, 2816, 1958, 1615, 1533 cm⁻¹.

### Step 2

1 g of the yellow crystalline solid obtained in step 1 of example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 911 mg; 91%) Anal calcd for C₅₀H₅₂N₁₁O₂ (single unit): C, 71.58; H, 6.25; N, 18.36. found: C, 71.72; H, 6.41; N, 18.22. IR (KBr); v: 3321, 2920, 2850, 1932, 1546, 1432 cm⁻¹.

### Example 30

### Step 1

A solution of (S)-(−)-1,1′-binaphthyl-2,2′-diamine (3.2 mmol, 910 mg in 10 ml of tetrahydrofuran) was added drop wise to another solution of white crystalline solid obtained in step 1 of example 25 (2 mmol, 1470 mg in 10 ml of tetrahydrofuran) and the resulting mixture was allowed to reflux at 85° C. for 8 h (checked on TLC). The resulting dark yellow solution was cooled to room temperature 25° C. followed by evaporation of solvent under vacuum. The yellow solid thus obtained was washed with methanol to get the desired product (Yield, 2.1 g; 95%). Anal calcd for C₅₀H₄₈N₁₁O₂ (single unit): C, 71.92, H, 5.79; N, 18.45. found: C, 72.05; H, 5.97; N, 18.28. IR (KBr); v: 3370, 2940, 2815, 1955, 1615, 1538 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 911 mg; 91%) Anal calcd for C₅₀H₅₂N₁₁O₂ (single unit): C, 71.58; H, 6.25; N, 18.36. found: C, 71.75; H, 6.43; N, 18.22. IR (KBr); v: 3311, 2928, 2856, 1950, 1548, 1434 cm⁻¹.

### Example 31

### Step 1

A solution of (R)-1,2 diaminopropane (3.2 mmol, 238 mg in 10 ml of tetrahydrofuran) was added drop wise to another solution of white crystalline solid obtained in step 1 of example 25 (2 mmol, 1470 mg in 10 ml of tetrahydrofuran) and the resulting mixture was allowed to reflux at 85° C. for 2 h (checked on TLC). The resulting dark yellow solution was cooled to room temperature 25° C. followed by evaporation of solvent under vacuum. The yellow solid thus obtained was washed with methanol to get the desired product (Yield, 1.52 g; 96%). Anal calcd for C₃₃H₄₂N₁₁O₂ (single unit): C, 63.44; H, 6.78; N, 24.66. found: C, 63.61; H, 6.93; N, 24.50. IR (KBr); v: 3320, 2934, 2820, 1942, 1626, 1550 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 902 mg; 90%) Anal calcd for C₃₃H₄₆N₁₁O₂ (single unit): C, 63.03; H, 7.37; N, 24.50. found: C, 63.21; H, 7.55; N, 24.32. IR (KBr); v: 3324, 2928, 2850, 2813, 2380, 1574 cm⁻¹.

### Example 32

### Step 1

A solution of (S)-1,2 diaminopropane (3.2 mmol, 238 mg in 10 ml of tetrahydrofuran) was added drop wise to another solution of white crystalline solid obtained in step 1 of example 25 (2 mmol, 1470 mg in 10 ml of tetrahydrofuran) and the resulting mixture was allowed to reflux at 85° C. for 2 h (checked on TLC). The resulting dark yellow solution was cooled to room temperature 25° C. followed by evaporation of solvent under vacuum. The yellow solid thus obtained was washed with methanol to get the desired product (Yield, 1.52 g; 96%). Anal calcd for C₃₃H₄₂N₁₁O₂ (single unit): C, 63.44; H, 6.78; N, 24.66. found: C, 63.60; H, 6.93; N, 24.50. IR (KBr); v: 3327, 2930, 2820, 1946, 1621, 1558 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 902 mg; 90%) Anal calcd for C₃₃H₄₆H₁₁O₂ (single unit): C, 63.03; H, 7.37; N, 24.50. found: C, 63.22; H, 7.46; N, 24.34. IR (KBr); v: 3324, 2936, 2852, 2801, 2382, 1574 cm⁻¹.

### Example 33

### Step 1

### 5,5′,5″-((4,4,4″-(1,3,5-triazine-2,4,6-triyl)tris(piperazine-4,1-diyl))tris(methylene))tris(2-hydroxy-3-methylbenzaldehyde)

White crystalline solid obtained in step 2 of example 1 (2 g, 6 mmol) was taken in 50 ml of dry toluene and a solution of 5-(chloromethyl)-2-hydroxy-3-methylbenzaldehyde (3.39 g, 18.4 mmol in 10 ml of dry toluene) was added to it. The resulting solution was refluxed at 110° C. for 8 h. The solid precipitated out was filtered and washed successively with toluene and diethyl ether. Afterwards the solid was taken in dichloromethane and neutralized with aqueous sodium bicarbonate solution. The organic phase was collected, dried over anhydrous sodium suphate followed by evaporation of the solvent leads to white crystalline solid (Yield, 4.3 g; 92%). Anal calcd for C₄₂H₅₁N₉O₆: C, 64.85; H, 6.61; N, 16.20. found: C, 64.91; H, 6.65; N, 16.13.

### Step 2

A solution of (1R,2R)-(−)-1,2-diaminocyclohexane (3.2 mmol, 366 mg in 10 ml of tetrahydrofuran) was added drop wise to another solution of white crystalline solid obtained in step 1 of this example (2 mmol, 1550 mg in 10 ml of tetrahydrofuran) and the resulting mixture was allowed to reflux at 85° C. for 2 h (checked on TLC). The resulting dark yellow solution was cooled to room temperature′ 25° C. followed by evaporation of solvent under vacuum. The yellow solid thus obtained was washed with methanol to get the desired product (Yield, 1.73 g; 97%). Anal calcd for C₃₈H₅₀N₁₁O₂ (single unit): C, 65.87; H, 7.27; N, 22.24. found: C, 66.02; H, 7.29; N, 22.12. IR (KBr); v: 3371, 2942, 2824, 1967, 1687, 1598 cm⁻¹.

### Step 3

1 g of the yellow crystalline solid obtained in step 2 of this example was dissolved in dry methanol:dichloromethane (4:1; 50 ml) to which 4 equivalents of sodium borohydride was added in 0.4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 911 mg; 91%) Anal calcd for C₃₈H₅₄N₁₁O₂ (single unit): C, 65.49; H, 7.81; N, 22.11. found: C, 65.65; H, 7.64; N, 22.00. IR (KBr); v: 3391, 2935, 2855, 2817, 2360, 1543, 1487 cm⁻¹.

### Example 34

### Step 1

A solution of (1S,2S)-(+)-1,2-diaminocyclohexane (3.2 mmol, 366 mg in 10 ml of tetrahydrofuran) was added drop wise to another solution of white crystalline solid obtained in step 1 of example 33 (2 mmol, 1550 mg in 10 ml of tetrahydrofuran) and the resulting mixture was allowed to reflux at 85° C. for 2 h (checked on TLC). The resulting dark yellow solution was cooled to room temperature 25° C. followed by evaporation of solvent under vacuum. The yellow solid thus obtained was washed with methanol to get the desired product (Yield, 1.73 g; 97%). Anal calcd for C₃₈H₅₀N₁₁O₂ (single unit): C, 65.87; H, 7.27; N, 22.24. found: C, 66.00; H, 7.39; N, 22.13. IR (KBr); v: 3328, 2944, 2808, 1953, 1628, 1533 cm⁻¹.

### Step 2

1 g of the yellow crystalline solid obtained in step 1 was dissolved in dry methanol:dichloromethane (4:1; 50 ml), then 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield 911 mg, 91%) Anal calcd for C₃₈H₅₄N₁₁O₂ (single unit): C, 65.49; H, 7.81; N, 22.11. found: C, 65.56; H, 7.66; N, 22.01. IR (KBr); v: 3351, 2939, 2838, 2827, 2326, 1547, 1489 cm⁻¹.

### Example 35

### Step 1

A solution of (1R,2R)-(+)-1,2-diphenylethylenediamine (3.2 mmol, 678 mg in 10 ml of tetrahydrofuran) was added drop wise to another solution of white crystalline solid obtained in step 1 of example 33 (2 mmol, 1550 mg in 10 ml of tetrahydrofuran) and the resulting mixture was allowed to reflux at 85° C. for 4 h (checked on TLC). The resulting dark yellow solution was cooled to room temperature 25° C. followed by evaporation of solvent under vacuum. The yellow solid thus obtained was washed with methanol to get the desired product (Yield, 1.98 g; 95%). Anal calcd for C₄₆H₅₂N₁₁O₂ (single unit): C, 69.85; H, 6.63; N, 19.48. found: C, 69.98; H, 6.78; N, 19.33. IR(KBr); v: 3659, 3385, 2946, 1952, 1626, 1530 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 901 mg; 90%) Anal calcd for C₄₆H₅₆N₁₁O₂ (single unit): C, 69.50; H, 7.10; N, 19.38. found: C, 69.70; H, 7.25; N, 19.23. IR(KBr); v: 3397, 2926, 2854, 2334, 1956, 1546, 1434, 1439 cm⁻¹.

### Example 36

### Step 1

A solution of (1S,2S)-(−)-1,2-diphenylethylenediamine (3.2 mmol, 678 mg in 10 ml of tetrahydrofuran) was added drop wise to another solution of white crystalline solid obtained in step 1 of example 33 (2 mmol, 1550 mg in 10 ml of tetrahydrofuran) and the resulting mixture was allowed to reflux at 85° C. for 4 h (checked on TLC). The resulting dark yellow solution was cooled to room temperature 25° C. followed by evaporation of solvent under vacuum. The yellow solid thus obtained was washed with methanol to get the desired product (Yield, 1.98 g; 95%). Anal calcd for C₄₆H₅₂N₁₁O₂ (single unit): C, 69.85; H, 6.63; N, 19.48. found: C, 69.98; H, 6.80; N, 19.38. IR(KBr); v: 3655, 3389, 2940, 1954, 1620, 1535 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 901 mg; 90%) Anal calcd for C₄₆H₅₆N₁₁O₂ (single unit): C, 69.50; H, 7.10; N, 19.38. found: C, 69.71; H, 7.28; N, 19.21. IR(KBr); v: 3309, 2927, 2854, 2362, 1952, 1546, 1480, 1436 cm⁻¹.

### Example 37

### Step 1

A solution of (R)-(+)-1,1′-binaphthyl-2,2′-diamine (3.2 mmol, 910 mg in 10 ml of tetrahydrofuran) was added drop wise to another solution of white crystalline solid obtained in step 1 of example 33 (2 mmol, 1550 mg in 10 ml of tetrahydrofuran) and the resulting mixture was allowed to reflux at 85° C. for 8 h (checked on TLC). The resulting dark yellow solution was cooled to room temperature 25° C. followed by evaporation of solvent under vacuum. The yellow solid thus obtained was washed with methanol to get the desired product (Yield, 2.16 g; 94%). Anal calcd for C₅₂H₅₂N₁₁O₂ (single unit): C, 72.37; H, 6.07; N, 17.85. found: C, 72.53; H, 6.24; N, 17.68. IR (KBr); v: 3374, 2948, 2818, 1956, 1610, 1538 cm⁻¹.

### Step 2

1 g of the yellow crystalline solid obtained in step 1 was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 898 mg; 90%) Anal calcd for C₅₂H₅₆N₁₁O₂ (single unit): C, 72.03; H, 6.51; N, 17.77. found: C, 72.23; H, 6.62; N, 17.58. IR (KBr); v: 3306, 2902, 2865, 1967, 1547, 1473 cm⁻¹.

### Example 38

### Step 1

A solution of (S)-(−)-1,1′-binaphthyl-2,2′-diamine (3.2 mmol, 910 mg in 10 ml of tetrahydrofuran) was added drop wise to another solution of white crystalline solid obtained in step 1 of example 33 (2 mmol, 1550 mg in 10 ml of tetrahydrofuran) and the resulting mixture was allowed to reflux at 85° C. for 8 h (checked on TLC). The resulting dark yellow solution was cooled to room temperature 25° C. followed by evaporation of solvent under vacuum. The yellow solid thus obtained was washed with methanol to get the desired product (Yield, 2.16 g; 94%). Anal calcd for C₅₂H₅₂N₁₁O₂ (single unit): C, 72.37; H, 6.07; N, 17.85. found: C, 72.49; H, 6.18; N, 17.69. IR (KBr); v: 3382, 2942, 2830, 1965, 1687, 1545 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 898 mg; 90%) Anal calcd for C₅₂H₅₆N₁₁O₂ (single unit): C, 72.03; H, 6.51; N, 17.77. found: C, 72.25; H, 6.60; N, 17.57. IR (KBr); v: 3300, 2998, 2845, 1974, 1587, 1436 cm⁻¹.

### Example 39

### Step 1

A solution of (R)-1,2 diaminopropane (3.2 mmol, 238 mg in 10 ml of tetrahydrofuran) was added drop wise to another solution of white crystalline solid obtained in step 1 of example 33 (2 mmol, 1550 mg in 10 ml of tetrahydrofuran) and the resulting mixture was allowed to reflux at 85° C. for 8 h (checked on TLC). The resulting dark yellow solution was cooled to room temperature 25° C. followed by evaporation of solvent under vacuum. The yellow solid thus obtained was washed with methanol to get the desired product (Yield, 1.6 g; 96%). Anal calcd for C₃₅H₄₆N₁₁O₂ (single unit): C, 64.39; H, 7.10; N, 23.60. found: C, 64.55; H, 7.20; N, 23.51. IR (KBr); v: 3375, 2934, 2823, 1987, 1629, 1545 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 904 mg; 90%) Anal calcd for C₃₅H₅₀N₁₁O₂ (single unit): C, 64.00; H, 7.67; N, 23.46. found: C, 64.21; H, 7.84; N, 23.31. IR (KBr); v: 3339, 2934, 2850, 2800, 2385, 1545 cm⁻¹.

### Example 40

### Step 1

A solution of (S)-1,2 diaminopropane (3.2 mmol, 238 mg in 10 ml of tetrahydrofuran) was added drop wise to another solution of white crystalline solid obtained in step 1 of example 33 (2 mmol, 1550 mg in 10 ml of tetrahydrofuran) and the resulting mixture was allowed to reflux at 85° C. for, 8 h (checked on TLC). The resulting dark yellow solution was cooled to room temperature 25° C. followed by evaporation of solvent under vacuum. The yellow solid thus obtained was washed with methanol to get the desired product (Yield, 1.6 g; 96%). Anal calcd for C₃₅H₄₆N₁₁O₂ (single unit): C, 64.39; H, 7.10; N, 23.60. found: C, 64.56; H, 7.14; N, 23.40. IR (KBr); v: 3356, 2934, 2823, 1998, 1645, 1554 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 904 mg; 90%) Anal calcd for C₃₅H₅₀N₁₁O₂ (single unit): C, 64.00; H, 7.67; N, 23.46. found: C, 64.16; H, 7.82; N, 23.32. IR (KBr); v: 3344, 2934, 2865, 2834, 2398, 1545 cm⁻¹.

### Example 41

### Step 1

### 5,5′,5″-(4″-(1,3,5-triazine-2,4,6-triyl)tris(piperazine-4,1-diyl))tris(methylene))tris(3-ethyl-2-hydroxybenzaldehyde)

White crystalline solid obtained in step 2 of example 1 (2 g, 6 mmol) was taken in 50 ml of dry toluene and a solution of 5-(chloromethyl)-3-ethyl-2-hydroxybenzaldehyde (3.64 g, 18.4 mmol in 10 ml of dry toluene) was added to it. The resulting solution was refluxed at 110° C. for 8 h. The solid precipitated out was filtered and washed successively with toluene and diethyl ether. Afterwards the solid was taken in dichloromethane and neutralized with aqueous sodium bicarbonate solution. The organic phase was collected, dried over anhydrous sodium suphate followed by evaporation of the solvent leads to white crystalline solid (Yield, 4.62 g; 94%). Anal calcd for C₄₅H₅₇N₉O₆: C, 65.91; H, 7.01; N, 15.37. found: C, 65.95; H, 7.04; N, 15.33.

### Step 2

A solution of (1R,2R)-(−)-1,2-diaminocyclohexane (3.2 mmol, 366 mg in 10 ml of tetrahydrofuran) was added drop wise to another solution of white crystalline solid obtained in step 1 of this example (2 mmol, 1640 mg in 10 ml of tetrahydrofuran) and the resulting mixture was allowed to reflux at 85° C. for 2 h (checked on TLC). The resulting dark yellow solution was cooled to room temperature 25° C. followed by evaporation of solvent under reduced pressure. The yellow solid thus obtained was washed with methanol to get the desired product (Yield, 1.88 g; 96%). Anal calcd for C₄₀H₅₄N₁₁O₂ (single unit): C, 66.64; H, 7.55; N, 21.37. found: C, 66.81; H, 7.68; N, 21.23. IR (KBr); v: 3370, 2940, 2817, 1950, 1624, 1533 cm⁻¹.

### Step 3

1 g of the yellow crystalline solid obtained in step 2 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under reduced pressure and the solid obtained was taken in dichloromethane, washed with water and finally evaporation of the solvent gives the desired product as white solid. (Yield, 918 mg; 92%). Anal calcd for C₄₀H₅₈N₁₁O₂ (single unit): C, 66.27; H, 8.06; N, 21.25. found: C, 66.41; H, 8.15; N, 21.06. IR (KBr); v: 3310, 2965, 2845, 2823, 2368, 1534, 1488 cm⁻¹.

### Example 42

### Step 1

A solution of (1S,2S)-(+)-1,2-diaminocyclohexane (3.2 mmol, 366 mg in 10 ml of tetrahydrofuran) was added drop wise to another solution of white crystalline solid obtained in step 2 of example 41 (2 mmol, 1640 mg in 10 ml of tetrahydrofuran) and the resulting mixture was allowed to reflux for 2 h (checked on TLC). The resulting dark yellow solution was cooled to room temperature 25° C. followed by evaporation of solvent under reduced pressure. The yellow solid thus obtained was washed with methanol to get the desired product (Yield, 1.88 g; 96%). Anal calcd for C₄₀H₅₄N₁₁O₂ (single unit): C, 66.64; H, 7.55; N, 21.37. found: C, 66.77; H, 7.61; N, 21.25. IR (KBr); v: 3356, 2949, 2845, 19223, 1629, 1539 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under reduced pressure and the solid obtained was taken in dichloromethane, washed with water and finally evaporation of the solvent gives the desired product as white solid. (Yield, 918 mg; 92%) Anal calcd for C₄₀H₅₈N₁₁O₂ (single unit): C, 66.27; H, 8.06; N, 21.25. found: C, 66.40; H, 8.12; N, 21.11. IR (KBr); v: 3311, 2936, 2845, 2837, 2356, 1534, 1434 cm⁻¹.

### Example 43

### Step 1

A solution of (1R,2R)-(+)-1,2-diphenylethylenediamine (3.2 mmol, 678 mg in 10 ml of tetrahydrofuran) was added drop wise to another solution of white crystalline solid obtained in step 2 of example 41 (2 mmol, 1640 mg in 10 ml of tetrahydrofuran) and the resulting mixture was allowed to reflux at 85° C. for 4 h (checked on TLC). The resulting dark yellow solution was cooled to room temperature 25° C. followed by evaporation of solvent under reduced pressure. The yellow solid thus obtained was washed with methanol to get the desired product (Yield, 2.06 g; 95%). Anal calcd for C₄₈H₅₆N₁₁O₂ (single unit): C, 70.39; H, 6.89; N, 18.81. found: C, 70.51; H, 6.99; N, 18.59. IR(KBr); v: 3671, 3331, 2946, 1955, 1626, 1530 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 908 mg; 91%) Anal calcd for C₄₈H₆₀N₁₁O₂ (single unit): C, 70.04; H, 7.35; N, 18.72. found: C, 70.20; H, 7.49; N, 18.59. IR(KBr); v: 3320, 2922, 2856, 2360, 1954, 1543, 1480, 1439 cm⁻¹.

### Example 44

### Step 1

A solution of (1S,2S)-(−)-1,2-diphenylethylenediamine (3.2 mmol, 678 mg in 10 ml of tetrahydrofuran) was added drop wise to another solution of white crystalline solid obtained in step 2 of example 41 (2 mmol, 1640 mg in 10 ml of tetrahydrofuran) and the resulting mixture was allowed to reflux at 85° C. for 4 h (checked on TLC). The resulting dark yellow solution was cooled to room temperature 25° C. followed by evaporation of solvent under vacuum. The yellow solid thus obtained was washed with methanol to get the desired product (Yield, 2.06 g; 95%). Anal calcd for C₄₈H₅₆N₁₁O₂ (single unit): C, 70.39; H, 6.89; N, 18.81. found: C, 70.55; H, 7.06; N, 18.69. IR(KBr); v: 3654, 3383, 2944, 1952, 1622, 1536 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 908 mg; 91%) Anal calcd for C₄₈H₆₀N₁₁O₂ (single unit): C, 70.04; H, 7.35; N, 18.72. found: C, 70.18; H, 7.48; N, 18.61. IR(KBr); v: 3300, 2924, 2858, 2360, 1955, 1543, 1480, 1435 cm⁻¹.

### Example 45

### Step 1

A solution of (R)-(+)-1,1′-binaphthyl-2,2′-diamine (3.2 mmol, 910 mg in 10 ml of tetrahydrofuran) was added drop wise to another solution of white crystalline solid obtained in step 2 of example 41 (2 mmol, 1640 mg in 10 ml of tetrahydrofuran) and the resulting mixture was allowed to reflux at 85° C. for 8 h (checked on TLC). The resulting dark yellow solution was cooled to room temperature 25° C. followed by evaporation of solvent under vacuum. The yellow solid thus obtained was washed with methanol to get the desired product (Yield, 2.19 g; 92%). Anal calcd for C₅₄H₅₆N₁₁O₂ (single unit): C, 72.78; H, 6.33; N, 17.29. found: C, 72.60; H, 6.48; N, 17.12. IR (KBr); v: 3370, 2946, 2816, 1955, 1610, 1563 cm⁻¹.

### Step 2

1 g of the yellow crystalline solid obtained in step 1 of this example was dissolved in dry methanol:dichloromethane (4:1, 50 ml) to which, 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 901 mg; 90%) Anal calcd for C₄₈H₆₀N₁₁O₂ (single unit): C, 72.46; H, 6.76; N, 17.21. found: C, 72.30; H, 6.66; N, 17.30. IR (KBr); v: 3381, 2928, 2859, 1955, 1544, 1439 cm⁻¹.

### Example 46

### Step 1

A solution of (S)-(−)-1,1′-binaphthyl-2,2′-diamine (3.2 mmol, 910 mg in 10 ml of tetrahydrofuran) was added drop wise to another solution of white crystalline solid obtained in step 2 of example 41 (2 mmol, 1640 mg in 10 ml of tetrahydrofuran) and the resulting mixture was allowed to reflux at 85° C. for 8 h (checked on TLC). The resulting dark yellow solution was cooled to room temperature 25° C. followed by evaporation of solvent under vacuum. The yellow solid thus obtained was washed with methanol to get the desired product (Yield, 2.19 g; 92%). Anal calcd for C₅₄H₅₆N₁₁O₂ (single unit): C, 72.78; H, 6.33; N, 17.29. found: C, 72.61; H, 6.44; N, 17.15. IR (KBr); v: 3370, 2940, 2815, 1954, 1612, 1534 cm⁻¹.

### Step 2

1 g of the yellow crystalline solid obtained in step 1 of the present example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 901 mg; 90%) Anal calcd for C₄₈H₆₀N₁₁O₂ (single unit): C, 72.46; H, 6.76; N, 17.21. found: C, 72.35; H, 6.67; N, 17.35. IR (KBr); v: 3311, 2928, 2854, 1945, 1544, 1463 cm⁻¹.

### Example 47

### Step 1

A solution of (R)-1,2 diaminopropane (3.2 mmol, 238 mg in 10 ml of tetrahydrofuran) was added drop wise to another solution of white crystalline solid obtained in step 2 of example 41 (2 mmol, 1640 mg in 10 ml of tetrahydrofuran) and the resulting mixture was allowed to reflux at 85° C. for 2 h (checked on TLC). The resulting dark yellow solution was cooled to room temperature 25° C. followed by evaporation of solvent under vacuum. The yellow solid thus obtained was washed with methanol to get the desired product (Yield, 1.68 g; 96%). Anal calcd for C₃₇H₅₀N₁₁O₂ (single unit): C, 65.27; H, 7.40; N, 22.63. found: C, 65.44; H, 7.58; N, 22.43. IR (KBr); v: 3320, 2930, 2822, 1946, 1620, 1550 cm⁻¹.

### Step 2

1 g of the yellow crystalline solid obtained in step 1 of the present example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 910 mg; 91%) Anal calcd for C₃₇H₅₀N₁₁O₂ (single unit): C, 64.89; H, 7.95; N, 22.50. found: C, 65.05; H, 8.13; N, 22.40. IR (KBr); v: 3324, 2928, 2846, 2813, 2334, 1517 cm⁻¹.

### Example 48

### Step 1

A solution of (S)-1,2-diaminopropane (3.2 mmol, 238 mg in 10 ml of tetrahydrofuran) was added drop wise to another solution of white crystalline solid obtained in step 2 of example 41 (2 mmol, 1640 mg in 10 ml of tetrahydrofuran) and the resulting mixture was allowed to reflux at 85° C. for 2 h (checked on TLC). The resulting dark yellow solution was cooled to room temperature 25° C. followed by evaporation of solvent under vacuum. The yellow solid thus obtained was washed with methanol to get the desired product (1.68 g, 96%). Anal calcd for C₃₇H₅₀N₁₁O₂ (single unit): C, 65.27; H, 7.40; N, 22.63. found: C, 65.48; H, 7.59; N, 22.45. IR (KBr); v: 3325, 2932, 2822, 1946, 1623, 1559 cm⁻¹.

### Step 2

1 g of the yellow crystalline solid obtained in step 1 of the present example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 910 mg; 91%) Anal calcd for C₃₇H₅₀N₁₁O₂ (single unit): C, 64.89; H, 7.95; N, 22.50. found: C, 65.11; H, 8.15; N, 22.32. IR (KBr); v: 3324, 2930, 2854, 2833, 2363, 1576 cm⁻¹.

### Example 49

### Step 1

### 5,5′,5″-((4,4′,4″-(1,3,5-triazine-2,4,6-triyl)tris(piperazine-4,1-diyl))tris(methylene))tris(2-hydroxy-3-isopropylbenzaldehyde)

White crystalline solid obtained in step 2 of example 1 (2 g, 6 mmol) was taken in 50 ml of dry toluene and a solution of 5-(chloromethyl)-2-hydroxy-3-isopropylbenzaldehyde (3.9 g, 18.4 mmol in 10 ml of dry toluene) was added to it. The resulting solution was refluxed at 110° C. for 8 h. The solid precipitated out was filtered and washed successively with toluene and diethyl ether. Afterwards the solid was taken in dichloromethane and neutralized with aqueous sodium bicarbonate solution. The organic phase was collected, dried over anhydrous sodium suphate followed by evaporation of the solvent leads to white crystalline solid (Yield, 4.86 g; 94%). Anal calcd for C₄₈H₆₃N₉O₆: C, 66.88; H, 7.37; N, 14.62. found: C, 66.92; H, 7.40; N, 14.58.

### Step 2

A solution of (1R,2R)-(−)-1,2-diaminocyclohexane (3.2 mmol, 366 mg in 10 ml of tetrahydrofuran) was added drop wise to another solution of white crystalline solid obtained in step 1 of this example (2 mmol, 1723 mg in 10 ml of tetrahydrofuran) and the resulting mixture was allowed to reflux at 85° C. for 2 h (checked on TLC). The resulting dark yellow solution was cooled to room temperature 25° C. followed by evaporation of solvent under vacuum. The yellow solid thus obtained was washed with methanol to get the desired product (Yield, 1.9 g; 97%). Anal calcd for C₄₂H₅₈N₁₁O₂ (single unit): C, 67.35; H, 7.81; N, 20.57. found: C, 67.55; H, 7.98; N, 20.42. IR (KBr); v: 3375, 2943, 2804, 1952, 1622, 1530 cm⁻¹.

### Step 3

1 g of the yellow crystalline solid obtained in step 2 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 919 mg; 92%) Anal calcd for C₄₂H₆₂N₁₁O₂ (single unit): C, 66.99; H, 8.30; N, 20.46. found: C, 67.15; H, 8.45; N, 20.29. IR (KBr); v: 3323, 2923, 2854, 2845, 2334, 1524, 1454 cm⁻¹.

### Example 50

### Step 1

A solution of (1S,2S)-(+)-1,2-diaminocyclohexane (3.2 mmol, 366 mg in 10 ml of tetrahydrofuran) was added drop wise to another solution of white crystalline solid obtained in step 1 of example 49 (2 mmol, 1723 mg in 10 ml of tetrahydrofuran) and the resulting mixture was allowed to reflux at 85° C. for 2 h (checked on TLC). The resulting dark yellow solution was cooled to room temperature 25° C. followed by evaporation of solvent under vacuum. The yellow solid thus obtained was washed with methanol to get the desired product (Yield, 1.9 g; 97%). Anal calcd for C₄₂H₅₈N₁₁O₂ (single unit): C, 67.35; H, 7.81; N, 20.57. found: C, 67.51; H, 7.92; N, 20.45. IR (KBr); v: 3348, 2940, 2817, 1954, 1624, 1512 cm⁻¹.

### Step 2

1 g of the yellow crystalline solid obtained in step 1 of this example was dissolved in dry methanol:dichloromethane (4:1; 50 ml), then 4 equivalents of sodium borohydride was added to it in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 919 mg; 92%) Anal calcd for C₄₂H₆₂N₁₁O₂ (single unit): C, 66.99; H, 8.30; N, 20.46. found: C, 67.15; H, 8.42; N, 20.28. IR (KBr); v: 3328, 2912, 2854, 2864, 2363, 1542, 1443 cm⁻¹.

### Example 51

### Step 1

A solution of (1R,2R)-(+)-1,2-diphenylethylenediamine (3.2 mmol, 678 mg in 10 ml of tetrahydrofuran) was added drop wise to another solution of white crystalline solid obtained in step 1 of example 49 (2 mmol, 1723 mg in 10 ml of tetrahydrofuran) and the resulting mixture was allowed to reflux at 85° C. for 4 h (checked on TLC). The resulting dark yellow solution was cooled to room temperature 25° C. followed by evaporation of solvent under vacuum. The yellow solid thus obtained was washed with methanol to get the desired product (Yield, 2.14 g; 95%). Anal calcd for C₅₀H₆₀N₁₁O₂ (single unit): C, 70.89; H, 7.14; N, 18.19. found: C, 71.05; H, 7.25; N, 18.09. IR(KBr); v: 3655, 3382, 2945, 1956, 1624, 1534 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 910 mg; 91%) Anal calcd for C₅₀H₆₄N₁₁O₂ (single unit): C, 70.56; H, 7.58; N, 18.10. found: C, 70.75; H, 7.67; N, 18.00. IR(KBr); v: 3323, 2923, 2858, 2365, 1954, 1534, 1453, 1432 cm⁻¹.

### Example 52

### Step 1

A solution of (1S,2S)-(−)-1,2-diphenylethylenediamine (3.2 mmol, 678 mg in 10 ml of tetrahydrofuran) was added drop wise to another solution of white crystalline solid obtained in step 1 of example 49 (2 mmol, 1723 mg in 10 ml of tetrahydrofuran) and the resulting mixture was allowed to reflux at 85° C. for 4 h (checked on TLC). The resulting dark yellow solution was cooled to room temperature 25° C. followed by evaporation of solvent under vacuum. The yellow solid thus obtained was washed with methanol to get the desired product (Yield, 2.14 g; 95%). Anal calcd for C₅₀H₆₀N₁₁O₂ (single unit): C, 70.89; H, 7.14; N, 18.19. found: C, 71.03; H, 7.22; N, 18.12. IR(KBr); v: 3321, 2922, 2858, 2362, 1953, 1542, 1480, 1433 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 910 mg; 91%) Anal calcd for C₅₀H₆₄N₁₁O₂ (single unit): C, 70.56; H, 7.58; N, 18.10. found: C, 70.78; H, 7.67; N, 17.96. IR (KBr); v: 3370, 2940, 2813, 1952, 1614, 1536 cm⁻¹.

### Example 53

### Step 1

A solution of (R)-(+)-1,1′-binaphthyl-2,2′-diamine (3.2 mmol, 910 mg in 10 ml of tetrahydrofuran) was added drop wise to another solution of white crystalline solid obtained in step 1 of example 49 (2 mmol, 1723 mg in 10 ml of tetrahydrofuran) and the resulting mixture was allowed to reflux at 85° C. for 8 h (checked on TLC). The resulting dark yellow solution was cooled to room temperature 25° C. followed by evaporation of solvent under vacuum. The yellow solid thus obtained was washed with methanol to get the desired product (Yield, 2.29 g; 93%). Anal calcd for C₅₆H₆₀N₁₁O₂ (single unit): C, 73.18; H, 6.58; N, 16.76. found: C, 73.39; H, 6.72; N, 16.55. IR (KBr); v: 3373, 2947, 2814, 1956, 1610, 1539 cm⁻¹.

### Step 2

1 g of the yellow crystalline solid obtained in step 1 of this example was dissolved in dry methanol:dichloromethane (4:1; 50 ml) with 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 901 mg; 90%) Anal calcd for C₅₆H₆₄N₁₁O₂ (single unit): C, 72.86; H, 6.99; N, 16.69. found: C, 73.10; H, 7.20; N, 16.55. IR (KBr); v: 3321, 2925, 2852, 1953, 1548, 1439 cm⁻¹.

### Example 54

### Step 1

A solution of (S)-(−)-1,1′-binaphthyl-2,2′-diamine (3.2 mmol, 910 mg in 10 ml of tetrahydrofuran) was added drop wise to another solution of white crystalline solid obtained in step 1 of example 49 (2 mmol, 1723 mg in 10 ml of tetrahydrofuran) and the resulting mixture was allowed to reflux at 85° C. for 8 h (checked on TLC). The resulting dark yellow solution was cooled to room temperature 25° C. followed by evaporation of solvent under vacuum. The yellow solid thus obtained was washed with methanol to get the desired product (Yield, 2.29 g; 93%). Anal calcd for C₅₆H₆₀N₁₁O₂ (single unit): C, 73.18; H, 6.58; N, 16.76. found: C, 73.35; H, 6.70; N, 16.59. IR (KBr); v: 3370, 2940, 2812, 1955, 1619, 1539 cm⁻¹.

### Step 2

1 g of the yellow crystalline solid obtained in step 1 of this example was dissolved in dry methanol:dichloromethane (4:1; 50 ml) with 4 equivalents of sodium borohydride was added to it in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 901 mg; 90%) Anal calcd for C₅₆H₆₄N₁₁O₂ (single unit): C, 72.86; H, 6.99; N, 16.69. found: C, 73.12; H, 7.20; N, 16.54. IR (KBr); v: 3303, 2925, 2859, 1959, 1548, 1438 cm⁻¹.

### Example 55

### Step 1

A solution of (R)-1,2-diaminopropane (3.2 mmol, 238 mg in 10 ml of tetrahydrofuran) was added drop wise to another solution of white crystalline solid obtained in step 1 of example 49 (2 mmol, 1723 mg in 10 ml of tetrahydrofuran) and the resulting mixture was allowed to reflux at 85° C. for 2 h (checked on TLC). The resulting dark yellow solution was cooled to room temperature 25° C. followed by evaporation of solvent under vacuum. The yellow solid thus obtained was washed with methanol to get the desired product (Yield, 1.76 g; 96%). Anal calcd for C₃₉H₅₄N₁₁O₂ (single unit): C, 66.07; H, 7.68; N, 21.73. found: C, 66.25; H, 7.83; N, 21.62. IR (KBr); v: 3324, 2930, 2822, 1941, 1623, 1550 cm⁻¹.

### Step 2

1 g of the yellow crystalline solid obtained in step 1 of this example was dissolved in dry methanol:dichloromethane (4:1; 50 ml) with 4 equivalents of sodium borohydride was added to it in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 909 mg; 91%) Anal calcd for C₃₉H₅₈N₁₁O₂ (single unit): C, 65.70; H, 8.20; N, 21.61. found: C, 65.92; H, 8.33; N, 21.44. IR (KBr); v: 3344, 2930, 28526, 2800, 2380, 1588 cm⁻¹.

### Example 56

### Step 1

A solution of (S)-1,2-diaminopropane (3.2 mmol, 238 mg in 10 ml of tetrahydrofuran) was added drop wise to another solution of white crystalline solid obtained in step 1 of example 49 (2 mmol, 1723 mg in 10 ml of tetrahydrofuran) and the resulting mixture was allowed to reflux at 85° C. for 2 h (checked on TLC). The resulting dark yellow solution was cooled to room temperature 25° C. followed by evaporation of solvent under vacuum. The yellow solid thus obtained was washed with methanol to get the desired product (Yield, 1.76 g; 96%). Anal calcd for C₃₉H₅₄N₁₁O₂ (single unit): C, 66.07; H, 7.68; N, 21.73. found: C, 66.22; H, 7.80; N, 21.65. IR (KBr); v: 3324, 2933, 2820, 1942, 1628, 1553 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 909 mg; 91%) Anal calcd for C₃₉H₅₈H₁₁O₂ (single unit): C, 65.70; H, 8.20; N, 21.61. found: C, 65.88; H, 8.33; N, 21.44. IR (KBr); v: 3324, 2934, 2858, 2801, 2381, 1578 cm⁻¹.

### Example 57

### Step 1

### 5,5′,5″-((4,4′,4″-(1,3,5-triazine-2,4,6-triyl)tris(piperazine-4,1-diyl))tris(methylene))tris(2-hydroxy-3-methoxybenzaldehyde)

White crystalline solid obtained in step 2 of example 1 (2 g, 6 mmol) was taken in 50 ml of dry toluene and a solution of 5-(chloromethyl)-2-hydroxy-3-methoxybenzaldehyde (3.7 g, 18.4 mmol in 10 ml of dry toluene) was added to it. The resulting solution was refluxed at 110° C. for 8 h. The solid precipitated out was filtered and washed successively with toluene and diethyl ether. Afterwards the solid was taken in dichloromethane and neutralized with aqueous sodium bicarbonate solution. The organic phase was collected, dried over anhydrous sodium suphate followed by evaporation of the solvent leads to white crystalline solid (Yield, 4.71 g; 95%). Anal calcd for C₄₂H₅₁N₉O₉: C, 61.08; H, 6.22; N, 15.26. found: C, 61.25; H, 6.33; N, 15.09.

### Step 2

A solution of (1R,2R)-(−)-1,2-diaminocyclohexane (3.2 mmol, 366 mg in 10 ml of tetrahydrofuran) was added drop wise to another solution of white crystalline solid obtained in step 1 of this example (2 mmol, 1652 mg in 10 ml of tetrahydrofuran) and the resulting mixture was allowed to reflux at 85° C. for 2 h (checked on TLC). The resulting dark yellow solution was cooled to room temperature 25° C. followed by evaporation of solvent under vacuum. The yellow solid thus obtained was washed with methanol to get the desired product (Yield, 1.83 g; 97%). Anal calcd for C₃₈H₅₀N₁₁O₄ (single unit): C, 62.96; H, 6.95; N, 21.26. found: C, 63.13; H, 7.03; N, 21.09. IR (KBr); v: 3372, 2943, 2809, 1952, 1628, 1530 cm⁻¹.

### Step 3

1 g of the yellow crystalline solid obtained in step 2 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 920; mg, 92%) Anal calcd for C₃₈H₅₄N₁₁O₄ (single unit): C, 62.62; H, 7.47; N, 21.14. found: C, 62.80; H, 7.60; N, 21.00. (KBr); v: 3316, 2930, 2854, 2800, 2362, 1523, 1454 cm⁻¹.

### Example 58

### Step 1

A solution of (1S,2S)-(+)-1,2-diaminocyclohexane (3.2 mmol, 366 mg in 10 ml of tetrahydrofuran) was added drop wise to another solution of white crystalline solid obtained in step 1 of example 57 (2 mmol, 1652 mg in 10 ml of tetrahydrofuran) and the resulting mixture was allowed to reflux at 85° C. for 2 h (checked on TLC). The resulting dark yellow solution was cooled to room temperature 25° C. followed by evaporation of solvent under vacuum. The yellow solid thus obtained was washed with methanol to get the desired product (Yield, 1.83 g, 97%). Anal calcd for C₃₈H₅₀N₁₁O₄ (single unit): C, 62.96; H, 6.95; N, 21.26. found: C, 63.13; H, 7.05; N, 21.10. IR (KBr); v: 3375, 2943, 2800, 1912, 1634, 1553 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 920 mg; 92%) Anal calcd for C₃₈H₅₄N₁₁O₄ (single unit): C, 62.62; H, 7.47; N, 21.14. found: C, 62.82; H, 7.59; N, 20.98. IR (KBr); v: 3315, 2943, 2855, 2835, 2335, 1565, 1475 cm⁻¹.

### Example 59

### Step 1

A solution of (1R,2R)-(+)-1,2-diphenylethylenediamine (3.2 mmol, 678 mg in 10 ml of tetrahydrofuran) was added drop wise to another solution of white crystalline solid obtained in step 1 of example 57 (2 mmol, 1652 mg in 10 ml of tetrahydrofuran) and the resulting mixture was allowed to reflux at 85° C. for 4 h (checked on TLC). The resulting dark yellow solution was cooled to room temperature 25° C. followed by evaporation of solvent under vacuum. The yellow solid thus obtained was washed with methanol to get the desired product (Yield, 2.05 g; 94%). Anal calcd for C₄₆H₅₂N₁₁O₄ (single unit): C, 67.13; H, 6.37; N, 18.72. found: C, 67.31; H, 6.46; N, 18.55. IR(KBr); v: 3653, 3385, 2943, 1955, 1624, 1534 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 912 mg; 91%) Anal calcd for C₄₆H₅₆N₁₁O₄ (single unit): C, 66.81; H, 6.83; N, 18.63. found: C, 67.00; H, 6.92; N, 18.45. IR(KBr); v: 3334, 2934, 2864, 2384, 1934, 1598, 1434, 1424 cm⁻¹.

### Example 60

### Step 1

A solution of (1S,2S)-(−)-1,2-diphenylethylenediamine (3.2 mmol, 678 mg in 10 ml of tetrahydrofuran) was added drop wise to another solution of white crystalline solid obtained in step 1 of example 57 (2 mmol, 1652 mg in 10 ml of tetrahydrofuran) and the resulting mixture was allowed to reflux at 85° C. for 4 h (checked on TLC). The resulting dark yellow solution was cooled to room temperature 25° C. followed by evaporation of solvent under vacuum. The yellow solid thus obtained was washed with methanol to get the desired product (Yield, 2.05 g; 94%). Anal calcd for C₄₆H₅₂N₁₁O₄ (single unit): C, 67.13; H, 6.37; N, 18.72. found: C, 67.34; H, 6.48; N, 18.52. IR(KBr); v: 3653, 3384, 2940, 1952, 1622, 1530 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 912 mg; 91%) Anal calcd for C₄₆H₅₆N₁₁O₄ (single unit): C, 66.81; H, 6.83; N, 18.63. found: C, 67.01; H, 6.92; N, 18.49. IR(KBr); v: 3323, 2943, 2852, 2354, 1925, 1564, 1464, 1446 cm⁻¹.

### Example 61

### Step 1

A solution of (R)-(+)-1,1′-binaphthyl-2,2′-diamine (3.2 mmol, 910 mg in 10 ml of tetrahydrofuran) was added drop wise to another solution of white crystalline solid obtained in step 1 of example 57 (2 mmol, 1652 mg in 10 ml of tetrahydrofuran) and the resulting mixture was allowed to reflux at 85° C. for 8 h (checked on TLC). The resulting dark yellow solution was cooled to room temperature 25° C. followed by evaporation of solvent under vacuum. The yellow solid thus obtained was washed with methanol to get the desired product (Yield, 2.2 g; 92%). Anal calcd for C₅₂H₅₂N₁₁O₄ (single unit): C, 69.78; H, 5.86; N, 17.21. found: C, 69.99; H, 5.97; N, 17.10. IR (KBr); v: 3370, 2941, 2834, 1924, 1614, 1554 cm⁻¹.

### Step 2

1 g of the yellow crystalline solid obtained in step 1 of this example was dissolved in dry, methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 902 mg; 90%) Anal calcd for C₅₂H₅₆N₁₁O₄ (single unit): C, 69.47; H, 6.28; N, 17.14. found: C, 69.66; H, 6.34; N, 17.00. IR (KBr); v: 3312, 2977, 2834, 1934, 1576, 1429 cm⁻¹.

### Example 62

### Step 1

A solution of (S)-(−)-1,1′-binaphthyl-2,2′-diamine (3.2 mmol, 910 mg in 10 ml of tetrahydrofuran) was added drop wise to another solution of white crystalline solid obtained in step 1 of example 57 (2 mmol, 1652 mg in 10 ml of tetrahydrofuran) and the resulting mixture was allowed to reflux at 85° C. for 8 h (checked on TLC). The resulting dark yellow solution was cooled to room temperature 25° C. followed by evaporation of solvent under vacuum. The yellow solid thus obtained was washed with methanol to get the desired product (Yield, 2.2 g; 92%). Anal calcd for C₅₂H₅₂N₁₁O₄ (single unit): C, 69.78; H, 5.86; N, 17.21. found: C, 70.01; H, 5.96; N, 17.05. IR (KBr); v: 3370, 2976, 2898, 1934, 1676, 1598 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 902 mg; 90%) Anal calcd for C₅₂H₅₆N₁₁O₄ (single unit): C, 69.47; H, 6.28; N, 17.14. found: C, 69.66; H, 6.38; N, 16.95. IR (KBr); v: 3300, 2923, 2857, 1965, 1576, 1487 cm⁻¹.

### Example 63

### Step 1

A solution of (R)-1,2-diaminopropane (3.2 mmol, 238 mg in 10 ml of tetrahydrofuran) was added drop wise to another solution of white crystalline solid obtained in step 1 of example 57 (2 mmol, 1652 mg in 10 ml of tetrahydrofuran) and the resulting mixture was allowed to reflux at 85° C. for 2 h (checked on TLC). The resulting dark yellow solution was cooled to room temperature 25° C. followed by evaporation of solvent under vacuum. The yellow solid thus obtained was washed with methanol to get the desired product (Yield, 1.71 g; 97%). Anal calcd for C₃₅H₄₆N₁₁O₄ (single unit): C, 61.39; H, 6.77; N, 22.50. found: C, 61.67; H, 6.88; N, 22.30. IR (KBr); v: 3318, 2923, 2854, 1964, 1624, 1565 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 921 mg; 92%) Anal calcd for C₃₅H₅₀N₁₁O₄ (single unit): C, 61.03; H, 7.32; N, 22.37. found: C, 61.22; H, 7.41; N, 22.22. IR (KBr); v: 3354, 2932, 2850, 2834, 2376, 1587 cm⁻¹.

### Example 64

### Step 1

A solution of (S)-1,2-diaminopropane (3.2 mmol, 238 mg in 10 ml of tetrahydrofuran) was added drop wise to another solution of white crystalline solid obtained in step 1 of example 57 (2 mmol, 1652 mg in 10 ml of tetrahydrofuran) and the resulting mixture was allowed to reflux at 85° C. for 2 h (checked on TLC). The resulting dark yellow solution was cooled to room temperature 25° C. followed by evaporation of solvent under vacuum. The yellow solid thus obtained was washed with methanol to get the desired product (Yield, 1.71 g; 97%). Anal calcd for C₃₅H₄₆N₁₁O₄ (single unit): C, 61.39; H, 6.77; N, 22.50. found: C, 61.64; H, 6.86; N, 22.35. IR (KBr); v: 3320, 2934, 2832, 1965, 1676, 1572 cm⁻¹.

### Step 2

1 g of the yellow crystalline solid obtained in step 1 of this example was dissolved in dry methanol:dichloromethane (4:1; 50 ml) to which 4 equivalents of sodium borohydride was added in 4 equal portions and the reaction mixture was allowed to stir at room temperature 25° C. for 10 h. After completion of the reaction, solvent was removed under vacuum and the solid obtained was taken in dichloromethane, washed with water and finally evaporation of the solvent gives the desired product as white solid. (Yield, 921 mg; 92%) Anal calcd for C₃₅H₅₀N₁₁O₄ (single unit): C, 61.03; H, 7.32; N, 22.37. found: C, 61.25; H, 7.43; N, 22.20. IR (KBr); v: 3354, 2987, 2824, 2823, 2317, 1534 cm⁻¹.

### Example 65-98

Asymmetric nitroaldol reactions of various aldehydes (0.5 mmol) with nitromethane (0.31 ml; 5.0 mmol) was carried out by in situ generated catalyst from representative but not limiting to ligands (as given in formulae 1) 1-22 (0.01 mmol) with Cu(OAc)₂.H₂O (0.01 mmol) at room temperature at 25° C. in a mixed solvent system comprising of ethanol:dichloromethane (1:1; 0.6 ml) in the presence of a base 2,6-luitidine (0.5 mmol) for 15-30 h. After the reaction was over the solvent was removed in vacuum and the residue was extracted with n-hexane (4×2 ml). The removal of hexane under vacuum and subsequent purification of the residue thus obtained by flash column chromatography on silica gel (100-200 mesh) using hexane:ethyl acetate (9:1) as eluent to get the desired product in yield and ee as mentioned in Table 1 against respective entry.

TABLE 1

| | | | | Yield | Ee |
|---|---|---|---|---|---|
| Examples | Aldehyde | Ligands | Base | (%) | (%) |
| 65 | 2-mehoxy benzaldehyde | Ligand1 | 2,6 | 75 | 79 |
| | | | lutidine | | |
| 66 | 3-methoxy benzaldehyde | Ligand1 | 2,6 | 91 | 92 |
| | | | lutidine | | |
| 67 | 4-methoxy benzaldehyde | Ligand1 | 2,6 | 82 | 90 |
| | | | lutidine | | |
| 68 | 4-chloro benzaldehyde | Ligand1 | 2,6 | 82 | 85 |
| | | | lutidine | | |
| 69 | 4-bromo benzaldehyde | Ligand1 | 2,6 | 76 | 92 |
| | | | lutidine | | |
| 70 | 4-fluoro benzaldehyde | Ligand1 | 2,6 | 86 | 85 |
| | | | lutidine | | |
| 71 | 4-trifluoro benzaldehyde | Ligand1 | 2,6 | 76 | 89 |
| | | | lutidine | | |
| 72 | 4-methyl benzaldehyde | Ligand1 | 2,6 | 84 | 76 |
| | | | lutidine | | |
| 73 | 4-nitro benzaldehyde | Ligand1 | 2,6 | 90 | 64 |
| | | | lutidine | | |
| 74 | cyclohexanal | Ligand1 | 2,6 | 90 | 92 |
| | | | lutidine | | |
| 75 | α-methyl | Ligand1 | 2,6 | 76 | 88 |
| | cinnamaldehyde | | lutidine | | |
| 76 | 1-hexanal | Ligand1 | 2,6 | 68 | 86 |
| | | | lutidine | | |
| 77 | 3,4- | Ligand1 | 2,6 | 90 | 96 |
| | dimethoxybenzaldehyde | | lutidine | | |
| 78 | 3,4- | Ligand2 | 2,6 | 78 | 91 |
| | dimethoxybenzaldehyde | | lutidine | | |
| 79 | 3,4- | Ligand3 | 2,6 | 98 | 95 |
| | dimethoxybenzaldehyde | | lutidine | | |
| 80 | 3,4- | Ligand4 | 2,6 | 84 | 88 |
| | dimethoxybenzaldehyde | | lutidine | | |
| 81 | 3,4- | Ligand5 | 2,6 | 92 | 90 |
| | dimethoxybenzaldehyde | | lutidine | | |
| 82 | 3,4- | Ligand6 | 2,6 | 68 | 91 |
| | dimethoxybenzaldehyde | | lutidine | | |
| 83 | 3,4- | Ligand7 | 2,6 | 55 | 78 |
| | dimethoxybenzaldehyde | | lutidine | | |
| 84 | 3,4- | Ligand8 | 2,6 | 45 | 65 |
| | dimethoxybenzaldehyde | | lutidine | | |
| 85 | 3,4- | Ligand9 | 2,6 | 55 | 92 |
| | dimethoxybenzaldehyde | | lutidine | | |
| 86 | 3,4- | Ligand10 | 2,6 | 50 | 65 |
| | dimethoxybenzaldehyde | | lutidine | | |
| 87 | 3,4- | Ligand11 | 2,6 | 65 | 70 |
| | dimethoxybenzaldehyde | | lutidine | | |
| 88 | 3,4- | Ligand12 | 2,6 | 60 | 68 |
| | dimethoxybenzaldehyde | | lutidine | | |
| 89 | 3,4- | Ligand13 | 2,6 | 64 | 70 |
| | dimethoxybenzaldehyde | | lutidine | | |
| 90 | 3,4- | Ligand14 | 2,6 | 60 | 88 |
| | dimethoxybenzaldehyde | | lutidine | | |
| 91 | 3,4- | Ligand15 | 2,6 | 68 | 90 |
| | dimethoxybenzaldehyde | | lutidine | | |
| 92 | 3,4- | Ligand16 | 2,6 | 63 | 70 |
| | dimethoxybenzaldehyde | | lutidine | | |
| 93 | 3,4- | Ligand17 | 2,6 | 54 | 67 |
| | dimethoxybenzaldehyde | | lutidine | | |
| 94 | 3,4- | Ligand18 | 2,6 | 64 | 85 |
| | dimethoxybenzaldehyde | | lutidine | | |
| 95 | 3,4- | Ligand19 | 2,6 | 60 | 80 |
| | dimethoxybenzaldehyde | | lutidine | | |
| 96 | 3,4- | Ligand20 | 2,6 | 44 | 92 |
| | dimethoxybenzaldehyde | | lutidine | | |
| 97 | 3,4- | Ligand21 | 2,6 | 62 | 82 |
| | dimethoxybenzaldehyde | | lutidine | | |
| 98 | 3,4- | Ligand22 | 2,6 | 56 | 76 |
| | dimethoxybenzaldehyde | | lutidine | | |

### Example 99

The in situ formed catalyst with ligand 1 and Cu(OAc)₂.H₂O in example 77 was recovered after the catalytic reaction was over by removing the solvent under vacuum and extracting the unreacted reactants and product with hexane (4×2 ml). The residue thus obtained was used directly without further purification to catalyze the nitroaldol reaction of 3,4-dimethoxybenzaldehyde (0.5 mmol) with nitromethane (5.0 mmol) under the reaction conditions mentioned in example 65 by adding fresh amount of 2,6-lutidine (0.5 mmol) in exactly the same manner as described in example 65 to get corresponding nitroalcohol in 90% yield and 96% ee.

### Example 100

The recovery of the catalyst from the catalytic run was done in the manner described in example 99 and was used again as catalyst in the nitroaldol of 3,4-dimethoxybenzaldehyde with nitromethane in exactly under the same condition as described in example 65 to get the corresponding nitroalcohol in 90% yield and 96% ee.

### Example 101

The recovery of the catalyst used in example 100 from the catalytic run was done in the manner described in example 99 and was used again as catalyst in the nitroaldol of 3,4-dimethoxybenzaldehyde with nitromethane in exactly under the same condition as described in example 65 to get the corresponding nitroalcohol in 89% yield and 95% ee.

### Example 102

The recovery of the catalyst used in example 101 from the catalytic run was done in the manner described in example 99 and was used again as catalyst in the nitroaldol of 3,4-dimethoxybenzaldehyde with nitromethane in exactly under the same condition as described in example 65 to get the corresponding nitroalcohol in 88% yield and 95% ee.

### Example 103

The recovery of the catalyst used in example 102 from the catalytic run was done in the manner described in example 99 and was used again as catalyst in the nitroaldol of 3,4 dimethoxybenzaldehyde with nitromethane in exactly under the same condition as described in example 65 to get the corresponding nitroalcohol in 88% yield and 95% ee.

### Example 104

This example constitutes results of 9 different catalytic experiments as tabulated in Table 2 for the asymmetric nitroaldol reaction of nitromethane (5 mmol) with 3,4-dimethoxybenzaldehyde (0.5 mmol) in the presence of different additives (a-d) (0.5 mmol) and different copper metal salts (e-j) (0.01 mmol) using ethanol:dichloromethane (1:1; 0.6 ml) as solvent at 25° C. under stirring for 25 h. These experiments were done in order to demonstrate the effect of additives and the effect of counter ion of the copper salts on activity and enantioselectivity in nitroaldol reaction. The best results were obtained in the case of nitroaldol of 3,4-dimethoxybenzaldehyde with nitromethane under the above reaction condition having materials as per entry 4 of this example.

TABLE 2
Catalytic Study of Mixture of Different Materials on Asymmetric
Nitroaldol Reaction.

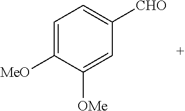 [SMILES: COc1ccc(C=O)cc1OC]

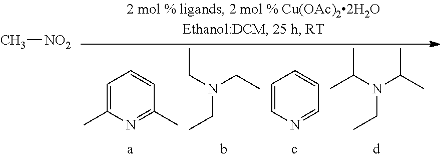

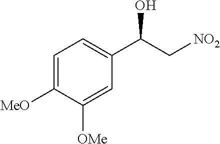 [SMILES: COc1ccc([C@@H](O)C[N+](=O)[O-])cc1OC]

| | | | | Yield | Ee |
|---|---|---|---|---|---|
| Entry | Ligands | Metal Source | Bases (additives) | (%) | (%) |
| 1 | Ligand 1 | Cu(OAc)₂•H₂O(e) | Pyridine (c) | 87 | 86 |
| 2 | Ligand 1 | Cu(OAc)₂•H₂O(e) | Triethylamine (b) | 95 | 70 |
| 3 | Ligand 1 | Cu(OAc)₂•H₂O(e) | N,N-diisopropylethy- | 80 | 76 |
| | | | amine(d) | | |
| 4 | Ligand 1 | Cu(OAc)₂•H₂O(e) | 2, 6-Lutidine (a) | 90 | 92 |
| 5 | Ligand 1 | Cu(OTf)₂(f) | 2, 6-Lutidine (a) | 74 | 78 |
| 6 | Ligand 1 | CuI(g) | 2, 6-Lutidine (a) | 56 | 60 |
| 7 | Ligand 1 | CuCl₂•2H₂O(h) | 2, 6-Lutidine (a) | 65 | 48 |
| 8 | Ligand 1 | Cu(NO₃)₂(i) | 2, 6-Lutidine (a) | 68 | 56 |
| 9 | Ligand 1 | CuBr(j) | 2, 6-Lutidine (a) | 55 | 65 |

### Example 105

This example was conducted in the same manner as given in example 77 except that the solvent used was as per the entry 1-4 given in Table 3. The respective results are given against each entry.

TABLE 3
Effect of Solvent on Asymmetric Nitroaldol Reaction of 3,4-
dimethoxybenzaldehyde.

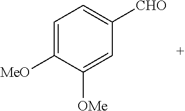 [SMILES: COc1ccc(C=O)cc1OC]

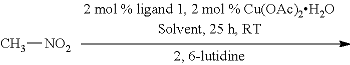

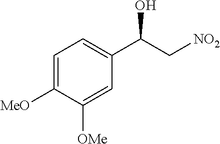 [SMILES: COc1ccc([C@@H](O)C[N+](=O)[O-])cc1OC]

| Entry | Solvent | Yield (%) | ee (%) |
|---|---|---|---|
| 1 | Tetrahydrofuran (THF) | 91 | 86 |
| 2 | Dichloromethane (DCM) | 92 | 80 |
| 3 | Diethylether (DEE) | 80 | 75 |
| 4 | Ethanol:DCM (1:1) | 90 | 95 |

### Example 106

This example was conducted in the same manner as given in example 77 except that the temperature used was as per the entries 1-4 given in Table 4. The respective results are given against each entry.

TABLE 4
Temperature Effect of Asymmetric Nitroaldol Reaction of 3,4-
dimethoxybenzaldehyde.

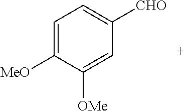 [SMILES: COc1ccc(C=O)cc1OC]

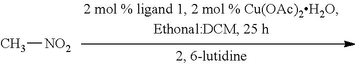

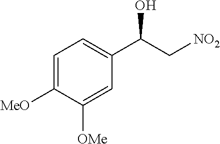 [SMILES: COc1ccc([C@@H](O)C[N+](=O)[O-])cc1OC]

| Entry | Temperature | Yield (%) | ee (%) |
|---|---|---|---|
| 1 | −10° C. | 65 | 95 |
| 2 | 0° C. | 70 | 95 |
| 3 | room temperature (27° C.) | 90 | 95 |
| 4 | 40° C. | 92 | 68 |

### Example 107

In this example, asymmetric nitroaldol reactions of 3,4-dimethoxybenzaldehyde (0.5 mmol) with nitromethane (0.31 ml; 5.0 mmol) was carried out by in situ generated catalyst from ligand 1 (0.01 mmol) with ZnEt₂ (0.01 mmol) at room temperature at 25° C. in a mixed solvent system comprising of ethanol:dichloromethane (1:1; 0.6 ml) in the presence of a base 2,6-lutidine (0.5 mmol) for 15-30 h in similar manner as described in example 145 to obtained the desired nitroaldol product in good yield (98%) but no enantioselectivity (ee, 21).

### Example 108

In this example, asymmetric nitroaldol reactions of 3,4-dimethoxybenzaldehyde (0.5 mmol) with nitromethane (0.31 ml; 5.0 mmol) was carried out by in situ generated catalyst from ligand 1 (0.01 mmol) with Co(OAc)₂.4H₂O (0.01 mmol) at room temperature at 25° C. in a mixed solvent system comprising of ethanol:dichloromethane (1:1; 0.6 ml) in the presence of a base 2,6-lutidine (0.5 mmol) for 15-30 h in similar manner as described in example 145 to obtained the desired nitroaldol product in good yield (95%) and enantioselectivity (ee, 45%).

### Example 109

In this example, asymmetric nitroaldol reactions of 3,4-dimethoxybenzaldehyde (0.5 mmol) with nitromethane (0.31 ml; 5.0 mmol) was carried out by in situ generated catalyst from ligand 1 (0.01 mmol) with CrCl₂.2H₂O (0.01 mmol) at room temperature at 25° C. in a mixed solvent system comprising of ethanol:dichloromethane (1:1; 0.6 ml) in the presence of a base 2,6-lutidine (0.5 mmol) for 15-30 h in similar manner as described in example 145 to obtained the desired nitroaldol product in good yield (93%) and enantioselectivity (ee, 36%).

### Example 110

In this example, asymmetric nitroaldol reactions of 3,4-dimethoxybenzaldehyde (0.5 mmol) with nitromethane (0.31 ml; 5.0 mmol) was carried out by in situ generated catalyst from ligands 1-11 (0.01 mmol) with Fe(acac)₃ (0.01 mmol) at room temperature at 25° C. in a mixed solvent system comprising of ethanol:dichloromethane (1:1; 0.6 ml) in the presence of a base 2,6-lutidine (0.5 mmol) for 15-30 h in similar manner as described in example 145 to obtained the desired nitroaldol product in good yield (92%) and enantioselectivity (ee, 25%).

### Example 111

In this example, asymmetric nitroaldol reactions of 3,4-dimethoxybenzaldehyde (0.5 mmol) with nitromethane (0.31 ml; 5.0 mmol) was carried out by in situ generated catalyst from ligands 1 (0.01 mmol) without any metal, as organocatalyst at room temperature at 25° C. in a mixed solvent system comprising of ethanol:dichloromethane (1:1; 0.6 ml) in the presence of a base 2,6-luitidine (0.5 mmol) for 15-30 h in similar manner as described in example 145 to obtained the desired nitroaldol product with 22% yield and 15% enantioselectivity.

### Example 112

Synthesis of (R)-Isopretenol—an non-selective β-adrenegenic agonist was accomplished by using asymmetric nitroaldol protocol used in the present invention in following steps; a) carrying out asymmetric nitroaldol of 3,4-dimethoxybenzaldehyde (5 mmol) with nitromethane (50 mmol) using the in situ generated complex from the ligand 1 (0.1 mmol) and Cu(OAc)₂.H₂O (0.1 mmol) and 2,6 lutidine (5 mmol) as base (yield, 90%); b) reduction of nitro group of the product obtained in step (a) of this example by H₂ gas using 10% Pd on C as catalyst (yield 95%); (c) condensing the product obtained from the step (b) with acetone to obtain corresponding Schiff base which on reduction with NaBH₄ followed by treatment of reduced product with BBr₃ to deprotect —OMe to —OH give the final product, (R)-Isopretenal in 92% yield and 95% ee.

### Example 113

Synthesis of (R)-Phenylephrine—a selective α₁-adrenergic receptor agonist was accomplished by using asymmetric nitroaldol protocol used in the present invention in following steps; a) carrying out asymmetric nitroaldol of 3-methoxybenzaldehyde (5 mmol) with nitromethane (50 mmol) using the in situ generated complex from the ligand 1 (0.1 mmol) and Cu(OAc)₂.H₂O (0.1 mmol) and 2,6-lutidine (5 mmol) as base yield 85%); b) reduction of nitro group of the product obtained in step (a) of this example by H₂ gas using 10% Pd on C as catalyst (yield 95%); (c) N-methytation of the product obtained from the step (b) with methyl iodide followed by treatment of methylated product with BBr₃ to demethylation of OMe to —OH give the final product, (R)-Phenylephrine in 90% yield and 94% ee.

### Example 114

Synthesis of (R)-Tembamide—an antiemetic was accomplished by using asymmetric nitroaldol protocol used in the present invention in following steps; a) carrying out asymmetric nitroaldol of 4-methoxybenzaldehyde (5 mmol) with nitromethane (50 mmol) using the in situ generated complex from the ligand 1 (0.1 mmol) and Cu(OAc)₂.H₂O (0.1 mmol) and 2,6-lutidine (5 mmol) as base (yield 92%); b) reduction of nitro group of the product obtained in step (a) of this example by H₂ gas using 10% Pd on C as catalyst (yield 95%); (c) benzoylation of the product obtained from the step (b) with benzoyl chloride to obtain the final product, (R)-Tembamide in 92% yield and 95% ee.

### Example 115

Synthesis of (R)-Ageline—an β-adregenic was accomplished by using asymmetric nitroaldol protocol used in the present invention in following steps; a) carrying out asymmetric nitroaldol of 4-methoxybenzaldehyde (5 mmol) with nitromethane (50 mmol) using the in situ generated complex from the ligand 1 (0.1 mmol) and Cu(OAc)₂.H₂O (0.1 mmol) and 2,6-lutidine (5 mmol) as base (yield 90%); b) reduction of nitro group of the product obtained in step (a) of this example by H₂ gas using 10% Pd on C as catalyst (yield 95%); (c) condensing the product obtained from the step (b) with cinnamoyl chloride to obtain the final product, (R)-Ageline in 85% yield and 94% ee.

### Example 116

Synthesis of (R)-norepherine—an neurotransmitter was accomplished by using asymmetric nitroaldol protocol used in the present invention in following steps; a) carrying out asymmetric nitroaldol of 3,4-dimethoxybenzaldehyde (5 mmol) with nitromethane (50 mmol) using the in situ generated complex from the ligand 1 (0.1 mmol) and Cu(OAc)₂.H₂O (0.1 mmol) and 2,6-lutidine (5 mmol) as base (yield 94%); b) reduction of nitro group of the product obtained in step (a) of this example by H₂ gas using 10% Pd on C as catalyst (yield 95%); (c) by treating the reduced product obtained in step (b) of this example with BBr₃ to demethylate —OMe to —OH to give the final product, (R)-norepherine in 91% yield and 97% ee.

### ADVANTAGES OF THE PRESENT INVENTION

The main advantages of the present invention are:
1. The chiral oligomeric [H₄]salen ligands used in the present invention are easily accessible and form highly active and enantioselective complex with Cu(OAc)₂.H₂O for the nitroaldol reaction of different types of aldehydes under homogeneous condition at ambient temperature, hence does not require additional heating or cooling device for doing catalysis.
2. The present nitroaldol protocol does not require additional chiral base in order to show high activity and enantioselectivity.
3. Although the catalyst was generated in situ and the catalytic run is performed under homogeneous condition the catalyst was effectively recycled and reused over several recycle experiments by simple solvent extraction method.
4. The present protocol required lowest catalyst loading reported so far for asymmetric nitroaldol reaction at ambient temperatures.
5. Due to the easy separation of the catalyst from the catalytic reaction mixture with hexane and ability to recycle the catalyst, the enantioselective nitroaldol reaction protocol disclosed in the present invention can be potentially used economically for the synthesis of pharmaceutically active compounds viz. R-phenylephrine, R-tembamide, R-aegiline, R-isoprotenolol and (R)-norepherine.

1. A chiral homogeneous catalyst comprising chiral ligand of general formula 1 along with metal 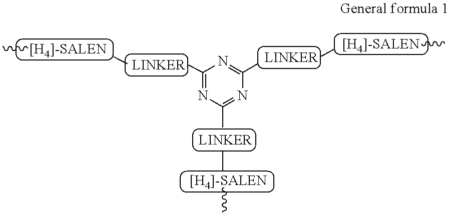
wherein
linker attached to melamine is selected from the group consisting of 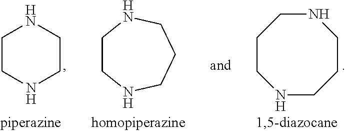
[H₄]salen attached to linker is selected from the group consisting of

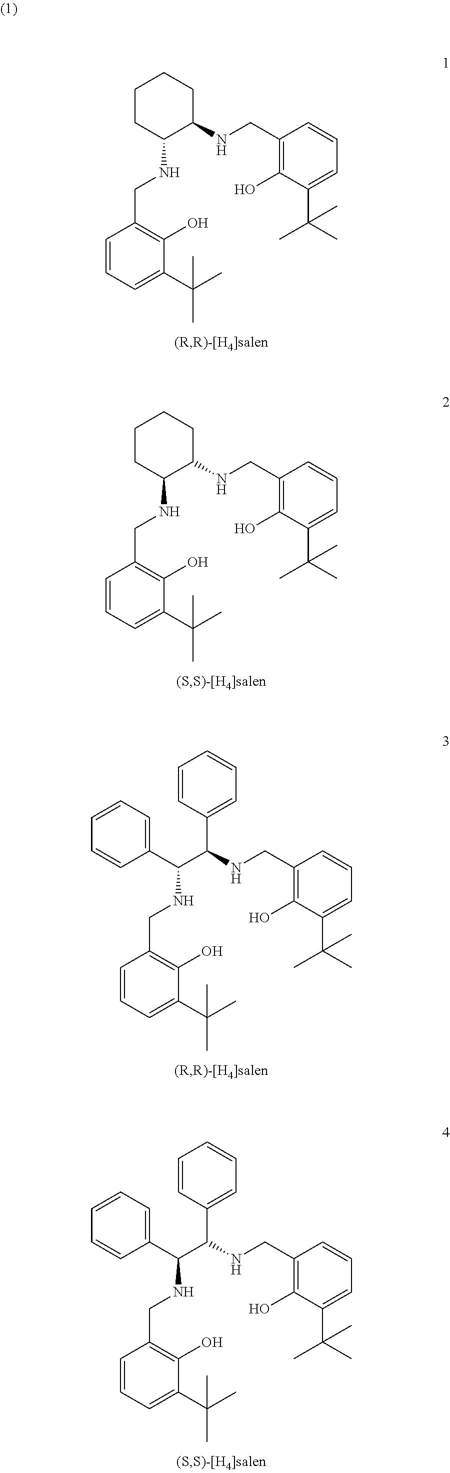

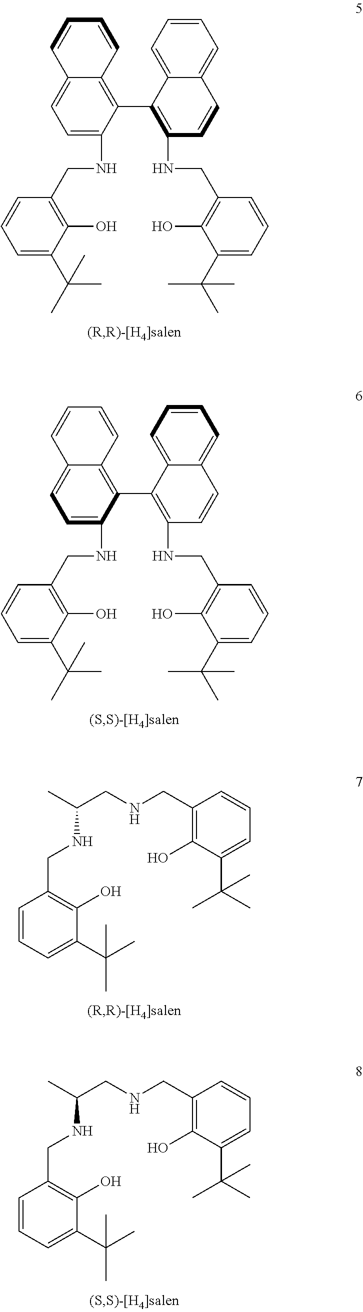

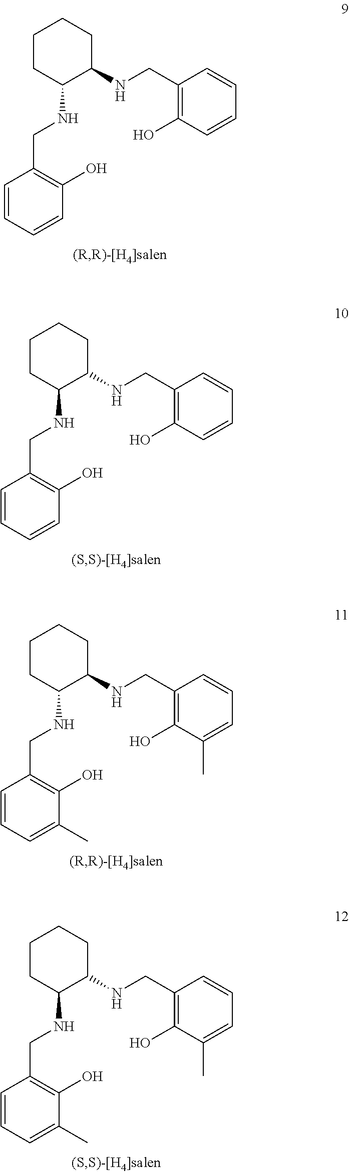

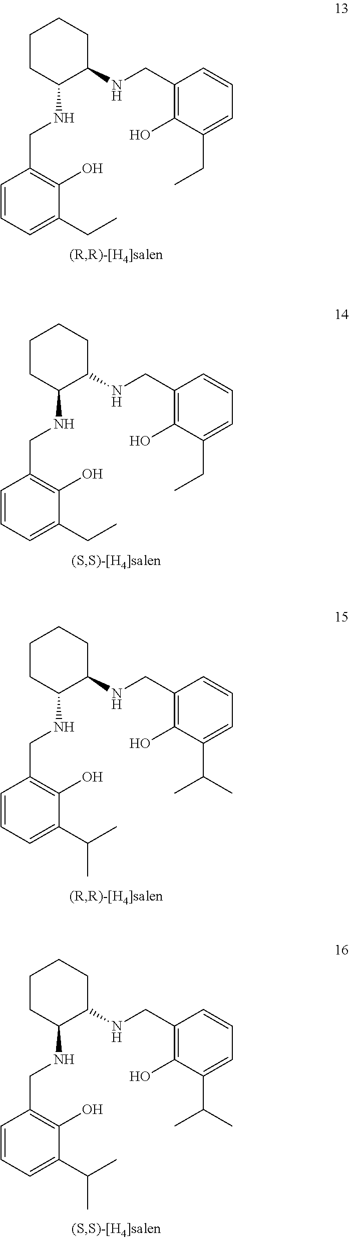

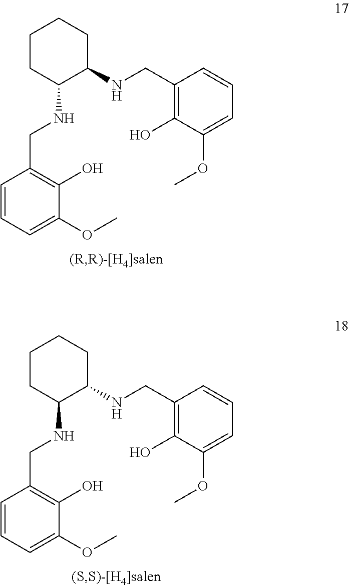 [SMILES: COc1cccc(CN[C@@H]2CCCC[C@H]2NCc2cccc(OC)c2O)c1O ; COc1cccc(CN[C@H]2CCCC[C@@H]2NCc2cccc(OC)c2O)c1O]

2. The chiral homogeneous catalyst as claimed in claim 1, wherein metal used is selected from the group consisting of cobalt(II), nickel (II), copper (I), copper (II) and Zn(II) preferably copper (II).

3. The chiral homogeneous catalyst as claimed in claim 1, wherein said catalyst is useful for asymmetric nitroaldol reaction for the synthesis of pharmaceutically important compounds.

4. The chiral homogeneous catalyst as claimed in claim 1, wherein chiral ligand of general formula 1 comprising:
piperazine: (1R,2R)—[H₄]salen 1; piperazine: (1S,2S)—[H₄]salen 2; piperazine: (1R,2R)—[H₄]salen 3; piperazine: (1S,2S)—[H₄]salen 4; piperazine: (R)—[H₄]salen 5; piperazine: (S)—[H₄]salen 6; piperazine: (R)—[H₄]salen 7; piperazine (S)—[H₄]salen 8; piperazine (1R,2R)—[H₄]salen 9; piperazine: (1S,2S)—[H₄]salen 10; piperazine: (1R,2R)—[H₄]salen 11; piperazine: (1S,2S)—[H₄]salen 12; piperazine: (1R,2R)-[H₄]salen 13; piperazine: (1S,2S)—[H₄]salen 14; piperazine: (1R,2R)—[H₄]salen 15; piperazine: (1S,2S)—[H₄]salen 16; piperazine: (1R,2R)—[H₄]salen 17; piperazine: (1S,2S)—[H₄]salen 18; homopiperazine: (1R,2R)—[H₄]salen 19; homopiperazine: (1S,2S)—[H₄]salen 20; 1,5-diazocane: (1R,2R)—[H₄]salen 21; 1,5-diazocane: (1S,2S)—[H₄]salen 22.

5. A process for the preparation of chiral ligand of formula 1 as claimed in claim 1 and the said process comprising the steps of:
i. reacting cyanuric chloride with a linker in the molar ratio ranging between 1:3 to 1:5 in the presence of 5 to 10 equivalent tertiary amine in dry tetrahydrofuran followed by refluxing under inert atmosphere for a period in the range of 12 to 24 h at temperature ranging from 65 to 66° C.;
ii. evaporating tetrahydrofuran from the reaction mixture as obtained from step (i) followed by extracting the solid thus obtained with dichloromethane, washing the dichloromethane layer with water, drying the dichloromethane layer with anhydrous sodium sulphate, evaporating of dichloromethane to give white solid and finally recrystalizing white solid from a mixture of dichloromethane and hexane (1:3);
iii. treating the white crystalline solid obtained from step (ii) with reagent in methanol in the molar ratio ranging between 1:20 to 1:40 at temperature in the range of 0 to 20° C. for period in the range of 3 to 8 h followed by keeping the reaction mixture at temperature in the range of 21 to 29° C. for period in the range of 12-24 h;
iv. removing methanol from the reaction mixture as obtained from step (iii) under vacuum followed by adding sodium hydroxide solution to make the pH of the solution at 14.0±1.0;
v. extracting aqueous layer as obtained from step (iv) with dichloromethane followed by removing dichloromethane under vacuum to obtain white solid;
vi. reacting the white solid obtained from step (v) with 3,4,6-(R3,R2,R1-substituted) 5-chloromethyl salicylaldehyde in the molar ratio ranging between 1:3 to 1:5 in dry toluene under reflux at temperature ranging between 110 to 120° C. for period in the range of 8-12 h to get a white crystalline solid;
vii. washing the white crystalline solid obtained from step (vi) with toluene and diethyl ether, dissolving the washed solid in dichloromethane, and washing the dichloromethane layer with aqueous sodium bicarbonate (10%);
viii. drying dichloromethane layer obtained from step (vii) over anhydrous sodium sulphate followed by filtration and removing dichloromethane from the filtrate gives white crystalline solid;
ix. treating the white crystalline solid obtained in step (viii) with a chiral 1,2-diamine in a molar ratio ranging between 1:1.5 to 1:3 in refluxed condition for period in the range of 2-10 h at temperature in the range of 65 to 66° C. in presence of dry tetrahydrofuran;
x. evaporating tetrahydrofuran from the solution obtained from step (ix) under vacuum to get yellow solid after washing with methanol and diethyl ether;
xi. treating the yellow solid obtained from step (x) with reducing agent in a molar ratio ranging between 1:4 to 1:8 in methanol at room temperature in the range of 25 to 27° C. for period in the range of 1 to 3 hr;
xii. evaporating methanol from the reaction mixture from step (xi), washing the solid obtained by dichloromethane and water to give chiral oligomeric [H₄]salen ligand of general formula 1.

6. The process as claimed in step (i) of claim 5, wherein linker used is selected from the group consisting of N-Boc piperazine, homopiperazine or 1,5-diazacane.

7. The process as claimed in step (i) of claim 5, wherein tertiary amine used is selected from the group consisting of triethylamine, triisopropylamine, N,N-diisopropylethylamine or 2,6-lutidine.

8. The process as claimed in step (iii) of claim 5, wherein reagent used for the removal of t-butoxycarbonyl group from N-protected linker is selected from the group consisting of trifluoroacetic acid (TFA), paratoluenesulfonic acid (PTSA), anhydrous alkali metal carbonate selected from sodium carbonate, potassium carbonate, rubidium carbonate and cesium carbonate, metallic sodium and inorganic mineral acid like hydrochloric acid (HCl).

9. The process as claimed in step (vi) of claim 5, wherein R₁, R₂, R₃ are selected from the group consisting of H, methyl, ethyl, propyl, isopropyl, n-butyl, isobutyl, t-butyl, or alkoxy selected from methoxy, ethoxy, butoxy or halogen selected from the group consisting of fluorine, chlorine, bromine and iodine.

10. The process as claimed in step (ix) of claim 5, wherein chiral 1,2-diamine used is selected from the group consisting of (S)-1,2-diaminopropane, (R)-1,2-diaminopropane, (1R,2R)-(−)-1,2-diaminocyclohexane, (1S,2S)-(+)-1,2-diaminocyclohexane, (1R,2R)-(+)-1,2-diphenyl-1,2-diaminoethane, (1S,2S)-(−)-1,2-diphenyl-1,2-diaminoethane, (R)-(+)-1,1′-binaphthyl-2,2′-diamine and (S)-(−)-1,1′-binaphthyl-2,2′-diamine.

11. The process as claimed in step (ix) of claim 5, wherein reducing agent used is selected from the group consisting of lithium aluminium hydride (LiAlH₄), sodium borohydride (NaBH₄), H₂/palladium-charcoal in an organic solvent selected from methanol, ethanol, n-propanol, iso-propanol, n-butanol, iso-butanol, tert-butanol, acetone, acetonitrile, propionitrile, toluene, xylene, diethylehter, tetrahydrofuran, dichloromethane and dichloroethane.

12. A process for preparation of nitroalcohol by asymmetric nitroaldol reactions using chiral homogeneous catalyst as claimed in claim 1 and the said process comprising the step of:
a) mixing 1 to 10 mol % of chiral ligand of general formula 1 in solvent;
b) adding the mixture as obtained in step (a) with metal salt in molar ratio ranging between 1:1 to 1:3 and with additive followed by stirring for period in the range of 1 to 3 h at a temperature ranging between 25-27° C. to generate in-situ active catalyst;
c) adding nitromethane and an aldehydes into the solution as obtained in step (b) and stirring the reaction mixture for a period ranging between 15 to 40 h preferably from 20-30 h at a temperature ranging between −20 to 110° C., preferably in the range of 0-60° C. more preferably from 10-30° C.;
d) evaporating solvent from the reaction mixture obtained from step (c) followed by repeatedly extracting by n-hexane and retrieving the solid by filtration/centrifugation;
e) evaporating the solvent from the combined filtrate as obtained in step (d) under vacuum to obtain crude nitroalcohol;
f) purifying the residue as obtained in step (e) by column chromatography using mixture of n-hexane:ethylacetate (90:10) to obtain 44 to 98% nitroaldol with 64-96% enantiomeric excess (ee).

13. The process as claimed in claim 12, wherein the solvent used in step (a) is selected from the group consisting of aliphatic hydrocarbons such as n-hexane, n-heptane, n-octane, cyclohexane; halogenated hydrocarbons such as dichloromethane, dichloroethane and carbon tetrachloride; aromatic hydrocarbons such as benzene, toluene, xylene, mesitylene, chloronenzene, nitrobenzene; ethers such as tetrahydrofuran, diethylether, tert-butylmethyl ether, cyclopentylmethyl ether and dimethoxyethane; alcohols such as methanol, ethanol, n-propanol, iso-propanol, n-butanol, iso-butanol and tert-butanol; esters such as methyl acetate, ethyl acetate and butyl acetate; nitriles such as acetonitrile, and butyronitrile; amides such as N,N-dimethylformamide, N,N-dimethylacetamide and N-methylpyrrolidone; and ureas such as tetramethylurea or combination thereof.

14. The process as claimed in claim 12, wherein the metal salt used in step (b) is selected from the group consisting of copper chloride, copper bromide, copper iodide, copper acetate, copper sulphate and copper triflate.

15. The process as claimed in step (b) of claim 12, wherein the additive used is selected from but not limiting to the group comprising of primary amine, secondary amine, tertiary amine, pyridine, 2-methyl pyridine, 2,6-lutidine, trimethylamine and triethylamine.

16. The process as claimed in step (c) of claim 12, wherein aldehydes used is selected from but not limiting to the group comprising of aromatic aldehyde, aliphatic aldehydes, α,β-unsaturated aldehydes and alicyclic aldehydes.

17. The process as claimed in claim 12, wherein the chiral homogeneous catalyst used in step (a) ranges between 0.5 to 50.0 mol %, preferably in the range of 1.0 to 35.0 mol % more preferably in the range of 5.0-20.0 mol % based on aldehydes.

18. The process as claimed in claim 12, wherein additive used in step (b) ranges between 1 to 40 mol %, preferably in the range of 1 to 10 mol % based on aldehyde.

19. The process as claimed in claim 12, wherein nitromethane used in step (c) is ranging between 200 to 1200 mol % with respect to aldehydes used.

20. The process as claimed in claim 12, wherein catalyst obtained in step (d) as solid is recyclable.
